(19) United States
(12) Patent Application Publication
(10) Pub. No.: US 2013/0143929 A1
(43) Pub. Date: Jun. 6, 2013

(54) FUNGICIDAL IMIDAZOLES

(71) Applicants: Jeffrey Keith Long, Wilmington, DE (US); James Francis Bereznak, Newtown Square, PA (US); Moumita Kar, Hyderabad (IN); Andrew Edmund Taggi, Newark, DE (US); Yuzhong Chen, Wilmington, DE (US)

(72) Inventors: Jeffrey Keith Long, Wilmington, DE (US); James Francis Bereznak, Newtown Square, PA (US); Moumita Kar, Hyderabad (IN); Andrew Edmund Taggi, Newark, DE (US); Yuzhong Chen, Wilmington, DE (US)

(73) Assignee: EI DU PONT DE NEMOURS AND COMPANY, Wilmington, DE (US)

(21) Appl. No.: 13/817,060

(22) PCT Filed: Sep. 28, 2011
(86) PCT No.: PCT/US11/53597

§ 371 Date: Feb. 14, 2013

(30) Foreign Application Priority Data
Sep. 29, 2010 (US) 61387671

(51) Int. Cl. A01N 43/50; C07D 401/04; C07D 233/64; C07D 409/04; A01N 43/56; C07D 233/68; C07D 403/04

(52) U.S. Cl. CPC A01N 43/50; C07D 233/68; C07D 401/04; C07D 403/04; C07D 409/04; A01N 43/56; C07D 233/64
USPC 514/341; 548/342.5; 514/400; 546/274.1

(57) ABSTRACT

Disclosed are compounds of Formula 1, including all stereoisomers, N-oxides, and salts thereof,

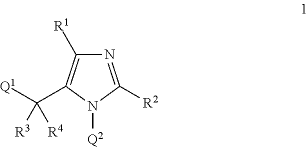

wherein
Q¹, Q², R¹, R², R³ and R⁴ are as defined in the disclosure.

Also disclosed are compositions containing the compounds of Formula 1 and methods for controlling plant disease caused by a fungal pathogen comprising applying an effective amount of a compound or a composition of the invention.

### FIELD OF THE INVENTION

This invention relates to certain imidazoles, their N-oxides, salts and compositions, and methods of their use as fungicides.

### BACKGROUND OF THE INVENTION

The control of plant diseases caused by fungal plant pathogens is extremely important in achieving high crop efficiency. Plant disease damage to ornamental, vegetable, field, cereal, and fruit crops can cause significant reduction in productivity and thereby result in increased costs to the consumer. Many products are commercially available for these purposes, but the need continues for new compounds which are more effective, less costly, less toxic, environmentally safer or have different sites of action.

PCT Patent Publication WO 2009/137651 discloses imidazole derivatives and their use as fungicides.

### SUMMARY OF THE INVENTION

This invention is directed to compounds of Formula 1 (including all stereoisomers), N-oxides, and salts thereof, agricultural compositions containing them and their use as fungicides:

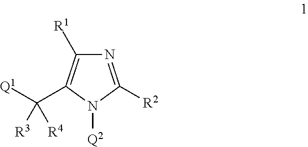

wherein
Q¹ is a phenyl ring substituted with 1 to 4 substituents independently selected from R^{5a}; or a thienyl, pyrazolyl, imidazolyl, thiazolyl, pyridinyl, pyridazinyl or pyrimidinyl ring or a quinazolinyl ring system, each ring or ring system optionally substituted with up to 4 substituents independently selected from R^{5a} on carbon atom ring members and R^{5b} on nitrogen atom ring members;
Q² is a phenyl ring substituted with 1 to 4 substituents independently selected from R^{5a}; or a thienyl, pyrazolyl, imidazolyl, thiazolyl, pyridinyl, pyridazinyl or pyrimidinyl ring or a quinazolinyl ring system, each ring or ring system optionally substituted with up to 4 substituents independently selected from R^{5a} on carbon atom ring members and R^{5b} on nitrogen atom ring members;
R¹ and R² are each independently H, halogen, cyano, nitro, C₁-C₃ alkyl, C₂-C₃ alkenyl, C₂-C₃ alkynyl, C₁-C₃ haloalkyl, C₂-C₃ haloalkenyl, cyclopropyl, halocyclopropyl, C₁-C₃ hydroxyalkyl, C₂-C₃ cyanoalkyl, C₂-C₃ alkoxyalkyl, C₁-C₃ alkoxy, C₁-C₃ haloalkoxy, C₁-C₃ alkylthio or C₁-C₃ haloalkylthio;
R³ is halogen, —OR⁶ or —SC≡N;
R⁴ is H or C₁-C₆ alkyl;
each R^{5a} is independently halogen, cyano, hydroxy, nitro, C₁-C₃ alkyl, C₂-C₃ alkenyl, C₂-C₃ alkynyl, C₁-C₃ haloalkyl, C₂-C₃ haloalkenyl, cyclopropyl, halocyclopropyl, C₂-C₃ cyanoalkyl, C₁-C₃ alkylthio, C₁-C₃ haloalkylthio, C₁-C₃ alkylsulfinyl, C₁-C₃ haloalkylsulfinyl, C₁-C₃ alkylsulfonyl, C₁-C₃ haloalkylsulfonyl, C₁-C₃ alkoxy, C₁-C₃ haloalkoxy, C₂-C₄ alkylcarbonyloxy, C₂-C₃ alkylcarbonyl, C₁-C₃ alkylamino, C₂-C₄ dialkylamino, C₂-C₃ alkylcarbonylamino, C₃-C₆ trialkylsilyl, —CH(═O), —NHCH(═O), —C(═S)NH₂, —SC≡N or -T-U—V;
each R^{5b} is independently cyano, C₁-C₃ alkyl, C₂-C₃ alkenyl, C₂-C₃ alkynyl, C₁-C₃ haloalkyl, cyclopropyl, C₂-C₃ alkoxyalkyl, C₂-C₃ alkylaminoalkyl, C₃-C₄ dialkylaminoalkyl, C₁-C₃ alkoxy, C₂-C₃ alkylcarbonyl or C₂-C₃ alkoxycarbonyl;
R⁶ is H, —CH(—O), C₁-C₆ alkyl, C₂-C₆ alkenyl, C₃-C₆ alkynyl, C₁-C₆ haloalkyl, C₃-C₆ cycloalkyl, C₃-C₆ halocycloalkyl, C₂-C₆ alkoxyalkyl, C₂-C₆ cyanoalkyl, C₂-C₆ alkylcarbonyl, C₂-C₆ alkoxycarbonyl, C₂-C₆ (alkylthio)carbonyl, C₄-C₈ cycloalkylcarbonyl, C₄-C₈ cycloalkoxycarbonyl, C₄-C₈ (cycloalkylthio)carbonyl, C₂-C₆ alkoxy(thiocarbonyl) or C₄-C₈ cycloalkoxy(thiocarbonyl);
each T is independently O, S(═O)ₙ, N(R⁷) or a direct bond;
each U is independently C₁-C₆ alkylene, C₂-C₆ alkenylene, C₃-C₆ alkynylene, C₃-C₆ cycloalkylene or C₃-C₆ cycloalkenylene, wherein up to 3 carbon atoms are independently selected from C(═O), each optionally substituted with up to 5 substituents independently selected from halogen, cyano, nitro, hydroxy, C₁-C₆ alkyl, C₁-C₆ haloalkyl, C₁-C₆ alkoxy and C₁-C₆ haloalkoxy;
each V is independently cyano, N(R^{8a})(R^{8b}), OR⁹ or S(═O)ₙR⁹;
each R⁷ is independently H, C₁-C₆ alkyl, C₁-C₆ haloalkyl, C₂-C₆ alkylcarbonyl, C₂-C₆ alkoxycarbonyl, C₂-C₆ (alkylthio)carbonyl, C₂-C₆ alkoxy(thiocarbonyl), C₄-C₈ cycloalkylcarbonyl, C₄-C₈ cycloalkoxycarbonyl, C₄-C₈ (cycloalkylthio)carbonyl or C₄-C₈ cycloalkoxy(thiocarbonyl);
each R^{8a} and R^{8b} is independently H, C₁-C₆ alkyl, C₁-C₆ haloalkyl, C₂-C₆ alkenyl, C₃-C₆ alkynyl, C₃-C₆ cycloalkyl, C₃-C₆ halocycloalkyl, C₂-C₆ alkylcarbonyl, C₂-C₆ alkoxycarbonyl, C₂-C₆ (alkylthio)carbonyl, C₂-C₆ alkoxy(thiocarbonyl), C₄-C₈ cycloalkylcarbonyl, C₄-C₈ cycloalkoxycarbonyl, C₄-C₈ (cycloalkylthio)carbonyl or C₄-C₈ cycloalkoxy(thiocarbonyl); or
a pair of R^{8a} and R^{8b} are taken together with the nitrogen atom to which they are attached to form a 4- to 7-membered heterocyclic ring, the ring optionally substituted with up to 5 substituents independently selected from R¹⁰;
each R⁹ is independently H, C₁-C₆ alkyl, C₁-C₆ haloalkyl, C₂-C₆ alkenyl, C₃-C₆ alkynyl, C₃-C₆ cycloalkyl, C₃-C₆ halocycloalkyl, C₂-C₆ alkylcarbonyl, C₂-C₆ alkoxycarbonyl, C₂-C₆ (alkylthio)carbonyl, C₂-C₆ alkoxy(thiocarbonyl), C₄-C₈ cycloalkylcarbonyl, C₄-C₈ cycloalkoxycarbonyl, C₄-C₈ (cycloalkylthio)carbonyl or C₄-C₈ cycloalkoxy(thiocarbonyl);
each R¹⁰ is independently halogen, C₁-C₆ alkyl, C₁-C₆ haloalkyl or C₁-C₆ alkoxy;
each n is independently 0, 1 or 2;
   provided that:
(a) when Q¹ and Q² are both phenyl substituted with 1 to 4 substituents independently selected from R^{5a}, then at least one R^{5a} substituent is attached at an ortho position; and
(b) when R¹ is H, then R² is other than H.

More particularly, this invention pertains to a compound selected from compounds of Formula 1 (including all stereoisomers) and N-oxides and salts thereof.

This invention also relates to a fungicidal composition comprising (a) a compound of the invention (i.e. in a fungicidally effective amount); and (b) at least one additional component selected from the group consisting of surfactants, solid diluents and liquid diluents.

This invention also relates to a fungicidal composition comprising (a) a compound of the invention; and (b) at least one other fungicide (e.g., at least one other fungicide having a different site of action).

This invention further relates to a method for controlling plant diseases caused by fungal plant pathogens comprising applying to the plant or portion thereof, or to the plant seed, a fungicidally effective amount of a compound of the invention (e.g., as a composition described herein).

This invention also relates to a composition comprising a compound of Formula 1, an N-oxide, or a salt thereof, and at least one invertebrate pest control compound or agent.

### DETAILS OF THE INVENTION

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a composition, mixture, process, method, article, or apparatus that comprises a list of elements is not necessarily limited to only those elements but may include other elements not expressly listed or inherent to such composition, mixture, process, method, article, or apparatus.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition, method or apparatus that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where applicants have defined an invention or a portion thereof with an open-ended term such as “comprising,” it should be readily understood that (unless otherwise stated) the description should be interpreted to also describe such an invention using the terms “consisting essentially of” or “consisting of.”

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As referred to in the present disclosure and claims, “plant” includes members of Kingdom Plantae, particularly seed plants (Spermatopsida), at all life stages, including young plants (e.g., germinating seeds developing into seedlings) and mature, reproductive stages (e.g., plants producing flowers and seeds). Portions of plants include geotropic members typically growing beneath the surface of the growing medium (e.g., soil), such as roots, tubers, bulbs and corms, and also members growing above the growing medium, such as foliage (including stems and leaves), flowers, fruits and seeds.

As referred to herein, the term “seedling”, used either alone or in a combination of words means a young plant developing from the embryo of a seed.

As referred to herein, the term “broadleaf” used either alone or in words such as “broadleaf crop” means dicot or dicotyledon, a term used to describe a group of angiosperms characterized by embryos having two cotyledons.

As used herein, the term “alkylating agent” refers to a chemical compound in which a carbon-containing radical is bound through a carbon atom to a leaving group such as halide or sulfonate, which is displaceable by bonding of a nucleophile to said carbon atom. Unless otherwise indicated, the term “alkylating agent” or “alkylating reagent” does not limit the carbon-containing radical to alkyl; the carbon-containing radicals in alkylating agents include the variety of carbon-bound substituent radicals specified, for example, for R¹ and R².

Generally when a molecular fragment (i.e. radical) is denoted by a series of atom symbols (e.g., C, H, N, O, S) the implicit point or points of attachment will be easily recognized by those skilled in the art. In some instances herein, particularly when alternative points of attachment are possible, the point or points of attachment may be explicitly indicated by a hyphen (“-”). For example, “—SC≡N” indicates that the point of attachment is the sulfur atom (i.e. thiocyanato, not isothiocyanato).

In the above recitations, the term “alkyl”, used either alone or in compound words such as “alkylthio” or “haloalkyl” includes straight-chain or branched alkyl such as methyl, ethyl, n-propyl, i-propyl, or the different butyl, pentyl or hexyl isomers. “Alkenyl” includes straight-chain or branched alkenes such as ethenyl, 1-propenyl, 2-propenyl, and the different butenyl, pentenyl and hexenyl isomers. “Alkenyl” also includes polyenes such as 1,2-propadienyl and 2,4-hexadienyl. “Alkynyl” includes straight-chain or branched alkynes such as ethynyl, 1-propynyl, 2-propynyl and the different butynyl, pentynyl and hexynyl isomers. “Alkynyl” can also include moieties comprised of multiple triple bonds such as 2,5-hexadiynyl. “Alkylene” denotes a straight-chain or branched alkanediyl. Examples of “alkylene” include CH₂, CH₂CH₂, CH(CH₃), CH₂CH₂CH₂, CH₂CH(CH₃), and the different butylene, pentylene or hexylene isomers. “Alkenylene” denotes a straight-chain or branched alkenediyl containing one olefinic bond. Examples of “alkenylene” include CH═CH, CH₂CH═CH and CH═C(CH₃). “Alkynylene” denotes a straight-chain or branched alkynediyl containing one triple bond. Examples of “alkynylene” include CH₂C≡C, C≡CCH₂, and the different butynylene, pentynylene or hexynylene isomers.

The term “cycloalkyl” denotes a saturated carbocyclic ring consisting of 3 to 6 carbon atoms linked to one another by single bonds. Examples of “cycloalkyl” include cyclopropyl, cyclobutyl, cyclopentyl and cyclohexyl. “Cycloalkylcarbonyl” denotes cycloalkyl bonded to a C(═O) group including, for example, cyclopropylcarbonyl and cyclopentylcarbonyl. The term “cycloalkoxycarbonyl” means cycloalkoxy bonded to a C(═O) group, for example, cyclopropyloxycarbonyl and cyclopentyloxycarbonyl. The term “cycloalkylene” denotes a cycloalkanediyl ring. Examples of “cycloalkylene” include cyclopropylene, cyclobutylene, cyclopentylene and cyclohexylene. The term “cycloalkenylene” denotes a cycloalkenediyl ring containing one olefinic bond. Examples of “cycloalkenylene” include cyclopropenylene and cyclopentenylene.

“Alkoxy” includes, for example, methoxy, ethoxy, n-propyloxy, i-propyloxy and the different butoxy, pentoxy and hexyloxy isomers. “Alkylthio” includes branched or straight-chain alkylthio moieties such as methylthio, ethylthio, and the different propylthio isomers. “Alkylsulfinyl” includes both enantiomers of an alkylsulfinyl group. Examples of “alkylsulfinyl” include CH₃S(═O), CH₃CH₂S(═O), CH₃CH₂CH₂S(═O) and (CH₃)₂CHS(═O). Examples of “alkylsulfonyl” include CH₃S(═O)₂, CH₃CH₂S(═O)₂, CH₃CH₂CH₂S(═O)₂ and (CH₃)₂CHS(═O)₂. “Alkylamino” includes an NH radical substituted with straight-chain or branched alkyl. Examples of “alkylamino” include CH₃CH₂NH, CH₃CH₂CH₂NH and (CH₃)₂CHNH. Examples of “dialkylamino” include (CH₃)₂N, (CH₃CH₂)₂N and CH₃CH₂(CH₃)N.

“Alkoxyalkyl” denotes alkoxy substitution on alkyl. Examples of “alkoxyalkyl” include CH₃OCH₂, CH₃OCH₂CH₂, CH₃CH₂OCH₂, CH₃CH₂CH₂CH₂OCH₂ and CH₃CH₂OCH₂CH₂. “Alkylaminoalkyl” denotes alkylamino substitution on alkyl. Examples of “alkylaminoalkyl” include CH₃NHCH₂, CH₃NHCH₂CH₂ and CH₃CH₂NHCH₂. Examples of “dialkylaminoalkyl” include (CH₃)₂NCH₂, CH₃CH₂(CH₃)NCH₂ and (CH₃)₂NCH₂CH₂.

“Cyanoalkyl” denotes an alkyl group substituted with one cyano group. Examples of “cyanoalkyl” include NCCH₂, NCCH₂CH₂ and CH₃CH(CN)CH₂. “Hydroxyalkyl” denotes an alkyl group substituted with one hydroxy group. Examples of “hydroxyalkyl” include HOCH₂, HOCH₂CH₂ and CH₃CH₂(OH)CH.

“Alkylcarbonyl” denotes a straight-chain or branched alkyl group bonded to a C(═O) moiety. Examples of “alkylcarbonyl” include CH₃C(═O), CH₃CH₂CH₂C(═O) and (CH₃)₂CHC(═O). Examples of “alkoxycarbonyl” include CH₃C(═O), CH₃CH₂OC(═O), CH₃CH₂CH₂C(═O), (CH₃)₂CHOC(═O) and the different pentoxy- or hexoxycarbonyl isomers. The term “alkylcarbonyloxy” denotes straight-chain or branched alkyl bonded to a C(═O)O moiety. Examples of “alkylcarbonyloxy” include CH₃CH₂C(═O)O and (CH₃)₂CHC(═O)O. “(Alkylthio)carbonyl” denotes a straight-chain or branched alkylthio group bonded to a C(═O) moiety. Examples of “(alkylthio)carbonyl” include CH₃SC(═O), CH₃CH₂CH₂SC(═O) and (CH₃)₂CHSC(═O). “Alkoxy(thiocarbonyl)” denotes a straight-chain or branched alkoxy group bonded to a C(═S) moiety. Examples of “alkoxy(thiocarbonyl)” include CH₃C(═S), CH₃CH₂CH₂C(═S) and (CH₃)₂CHOC(═S). The term “alkylcarbonylamino” denotes alkyl bonded to a C(═O)NH moiety. Examples of “alkylcarbonylamino” include CH₃C(═O)NH and CH₃CH₂C(═O)NH.

“Trialkylsilyl” includes 3 branched and/or straight-chain alkyl radicals attached to and linked through a silicon atom, such as trimethylsilyl, triethylsilyl and tert-butyldimethylsilyl.

The term “halogen”, either alone or in compound words such as “halomethyl”, “haloalkyl”, includes fluorine, chlorine, bromine or iodine. Further, when used in compound words such as “haloalkyl”, said alkyl may be partially or fully substituted with halogen atoms which may be the same or different. Examples of “haloalkyl” include F₃C, ClCH₂, CF₃CH₂ and CF₃CCl₂. The terms “haloalkenyl”, “haloalkoxy”, “haloalkylthio”, “haloalkylsulfinyl” “haloalkylsulfonyl”, “halocycloalkyl” and “halocycloalkyl” are defined analogously to the term “haloalkyl”. Examples of “haloalkenyl” include Cl₂C═CHCH₂ and CF₃CH₂═CH. Examples of “haloalkoxy” include CF₃O, CCl₃CH₂O, F₂CHCH₂CH₂O and CF₃CH₂O. Examples of “haloalkylthio” include CCl₃S, CF₃S, CCl₃CH₂S and ClCH₂CH₂CH₂S. Examples of “haloalkylsulfinyl” include CF₃S(═O), CCl₃S(═O), CF₃CH₂S(═O) and CF₃CF₂S(═O). Examples of “haloalkylsulfonyl” include CF₃S(═O)₂, CCl₃S(═O)₂, CF₃CH₂S(═O)₂ and CF₃CF₂S(═O)₂. Examples of “halocycloalkyl” include chlorocyclopropyl, fluorocyclobutyl and chlorocyclohexyl.

The total number of carbon atoms in a substituent group is indicated by the “Cᵢ-Cⱼ” prefix where i and j are numbers from 1 to 8. For example, C₁-C₃ alkylsulfonyl designates methylsulfonyl through propylsulfonyl; C₂ alkoxyalkyl designates CH₃OCH₂; C₃ alkoxyalkyl designates, for example, CH₃OCH₂CH₂ or CH₃CH₂OCH₂; and C₄ alkoxyalkyl designates the various isomers of an alkyl group substituted with an alkoxy group containing a total of four carbon atoms, examples including CH₃CH₂CH₂OCH₂ and CH₃CH₂OCH₂CH₂.

The term “unsubstituted” in connection with a group such as a ring means the group does not have any substituents other than its one or more attachments to the remainder of Formula 1. The term “optionally substituted” means that the number of substituents can be zero. Unless otherwise indicated, optionally substituted groups may be substituted with as many optional substituents as can be accommodated by replacing a hydrogen atom with a non-hydrogen substituent on any available carbon or nitrogen atom. Commonly, the number of optional substituents (when present) range from 1 to 3. As used herein, the term “optionally substituted” is used interchangeably with the phrase “substituted or unsubstituted” or with the term “(un)substituted.”

The number of optional substituents may be restricted by an expressed limitation. For example, the phrase “optionally substituted with up to 4 substituents independently selected from R^{5a} on carbon atom ring members” means that 0, 1, 2, 3 or 4 substituents can be present (if the number of potential connection points allows).

Unless otherwise indicated, a “ring” or “ring system” as a component of Formula 1 (e.g., Q²) is carbocyclic (e.g., phenyl) or heterocyclic (e.g., pyridinyl). The term “ring member” refers to an atom (e.g., C, O, N or S) forming the backbone of a ring. The term “ring system” denotes two or more fused rings (e.g., quinazolinyl).

The term “nonaromatic” includes rings that are fully saturated as well as partially or fully unsaturated, provided that none of the rings are aromatic. The term “aromatic” indicates that each of the ring atoms of a fully unsaturated ring are essentially in the same plane and have a p-orbital perpendicular to the ring plane, and that (4n+2) it electrons, where n is a positive integer, are associated with the ring to comply with Hückel's rule.

The terms “carbocyclic ring” or “carbocycle” denote a ring wherein the atoms forming the ring backbone are selected only from carbon. When a fully unsaturated carbocyclic ring satisfies Hückel's rule, then said ring is also called an “aromatic carbocyclic ring”. The term “saturated carbocyclic ring” refers to a ring having a backbone consisting of carbon atoms linked to one another by single bonds; unless otherwise specified, the remaining carbon valences are occupied by hydrogen atoms.

The terms “heterocyclic ring”, “heterocycle” or “heterocyclic ring system” denote a ring or ring system in which at least one atom forming the ring backbone is not carbon (e.g., N, O or S). Typically a heterocyclic ring contains no more than 3 N atoms, no more than 2 O atoms and no more than 2 S atoms. Unless otherwise indicated, a heterocyclic ring can be a saturated, partially unsaturated or fully unsaturated ring. When a fully unsaturated heterocyclic ring satisfies Hückel's rule, then said ring is also called a “heteroaromatic ring” or “aromatic heterocyclic ring”. Unless otherwise indicated, heterocyclic rings can be attached through any available carbon or nitrogen by replacement of a hydrogen on said carbon or nitrogen.

In the context of the present invention when an instance of Q¹ and Q² comprises a phenyl or 6-membered heterocyclic ring (e.g., pyridinyl), the ortho, meta and para positions of each ring is relative to the connection of the ring to the remainder of Formula 1.

As noted above, Q¹ and Q² can be, inter alia, a phenyl ring substituted with 1 to 4 substituents independently selected from R^{5a}. When an instance of Q¹ or Q² comprises a phenyl ring substituted with 4 or less R^{5a} substituents, then hydrogen atoms are attached to take up any free valency.

Compounds of this invention can exist as one or more stereoisomers. The various stereoisomers include enantiomers, diastereomers, atropisomers and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. The compounds of the invention may be present as a mixture of stereoisomers, individual stereoisomers or as an optically active form. Of note are atropisomers, which are stereoisomeric conformations of a molecule that occur when rotation about a single bond is restricted such that interconversion is slow enough to allow separation. Restricted rotation of one or more bonds is a result of steric interaction with other parts of the molecule. In the present invention, compounds of Formula 1 can exhibit atropisomerism when the energy barrier to free rotation around a single unsymmetrical bond is sufficiently high that separation of isomers is possible. Atropisomerism is defined to exist where the isomers have a half-life of at least 1000 seconds, which is a free energy barrier of at least about 22.3 kcal mol⁻¹ at about 20° C. (Oki, Topics in Stereochemistry, Vol. 14, John Wiley & Sons, Inc., 1983). One skilled in the art will appreciate that one atropisomer may be more active and/or may exhibit beneficial effects when enriched relative to other atropisomers or when separated from other atropisomers. Additionally, the skilled artisan knows how to separate, enrich, and/or to selectively prepare said atropisomers. Further description of atropisomers can be found in March, Advanced Organic Chemistry, 101-102, 4^{th} Ed. 1992; Oki, Topics in Stereochemistry, Vol. 14, John Wiley & Sons, Inc., 1983 and Gawronski et al, Chirality 2002, 14, 689-702. This invention comprises enriched mixtures and essentially pure atropisomers of compounds of Formula 1.

Also of note are enantiomers of Formula 1. For example, two possible enantiomers of Formula 1 are depicted below as Formula 1′ and Formula 1″ wherein the chiral center is identified with an asterisk (*) and the substituents R³ and R⁴ are not identical.

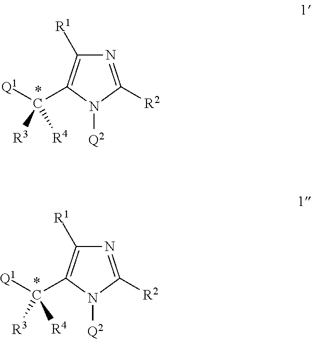

Molecular depictions drawn herein follow standard conventions for depicting stereochemistry. To indicate stereoconfiguration, bonds rising from the plane of the drawing and towards the viewer are denoted by solid wedges wherein the broad end of the wedge is attached to the atom rising from the plane of the drawing towards the viewer. Bonds going below the plane of the drawing and away from the viewer are denoted by dashed wedges wherein the narrow end of the wedge is attached to the atom further away from the viewer. Constant width lines indicate bonds with a direction opposite or neutral relative to bonds shown with solid or dashed wedges; constant width lines also depict bonds in molecules or parts of molecules in which no particular stereoconfiguration is intended to be specified.

This invention comprises racemic mixtures, for example, equal amounts of the enantiomers of Formulae 1′ and 1″. In addition, this invention includes compounds that are enriched compared to the racemic mixture in an enantiomer of Formula 1. Also included are the essentially pure enantiomers of compounds of Formula 1, for example, Formula 1′ and Formula 1″.

When enantiomerically enriched, one enantiomer is present in greater amounts than the other, and the extent of enrichment can be defined by an expression of enantiomeric excess (“ee”), which is defined as (2x−1)·100%, where x is the mole fraction of the dominant enantiomer in the mixture (e.g., an ee of 20% corresponds to a 60:40 ratio of enantiomers).

Of note are compositions of this invention having at least a 50%, or at least a 75%, or at least a 90%, or at least a 94% enantiomeric excess of an isomer. Of particular note are enantiomerically pure embodiments.

Compounds of Formula 1 can comprise additional chiral centers. For example, substituents such as R^{5a} may themselves contain chiral centers.

One skilled in the art recognizes that because in the environment and under physiological conditions salts of chemical compounds are in equilibrium with their corresponding nonsalt forms, salts share the biological utility of the nonsalt forms. Thus a wide variety of salts of the compounds of Formula 1 are useful for control of plant diseases caused by fungal plant pathogens (i.e. are agriculturally suitable). The salts of the compounds of Formula 1 include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids.

Compounds selected from Formula 1, stereoisomers, N-oxides, and salts thereof, typically exist in more than one form, therefore Formula 1 includes all crystalline and non-crystalline forms of the compounds that Formula 1 represents. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e. different crystalline types). The term “polymorph” refers to a particular crystalline form of a chemical compound that can crystallize in different crystalline forms, these forms having different arrangements and/or conformations of the molecules in the crystal lattice. Although polymorphs can have the same chemical composition, they can also differ in composition due to the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound represented by Formula 1 can exhibit beneficial effects (e.g., suitability for preparation of useful formulations, improved biological performance) relative to another polymorph or a mixture of polymorphs of the same compound represented by Formula 1. Preparation and isolation of a particular polymorph of a compound represented by Formula 1 can be achieved by methods known to those skilled in the art including, for example, crystallization using selected solvents and temperatures.

Embodiments of the present invention as described in the Summary of the Invention include those described below. In the following Embodiments, Formula 1 includes stereoisomers, N-oxides and salts thereof, and reference to “a compound of Formula 1” includes the definitions of substituents specified in the Summary of the Invention unless further defined in the Embodiments.

### Embodiment 1

A compound of Formula 1 wherein Q¹ is a phenyl ring substituted with 1 to 3 substituents independently selected from R^{5a}; or a pyridinyl or pyrimidinyl ring optionally substituted with up to 3 substituents independently selected from R^{5a}.

### Embodiment 2

A compound of Embodiment 1 wherein Q¹ is a phenyl ring substituted with 1 to 3 substituents independently selected from R^{5a}.

### Embodiment 3

A compound of Embodiment 2 wherein Q¹ is a phenyl ring substituted with 3 substituents independently selected from R^{5a}.

### Embodiment 4

A compound of Embodiment 3 wherein Q¹ is a phenyl ring substituted with 2 substituents independently selected from R^{5a}.

### Embodiment 5

A compound of Formula 1 or any one of Embodiments 1 through 4 wherein Q¹ is a phenyl ring substituted with at least one R^{5a} substituent attached at an ortho position (relative to the connection of the Q¹ ring to the remainder of Formula 1).

### Embodiment 6

A compound of Formula 1 or any one of Embodiments 1 through 5 wherein Q² is a phenyl ring substituted with 1 to 3 substituents independently selected from R^{5a}; or a pyrazolyl, pyridinyl or pyrimidinyl ring optionally substituted with up to 3 substituents independently selected from R^{5a} on carbon atom ring members and methyl on the nitrogen atom ring member.

### Embodiment 7

A compound of Embodiment 6 wherein Q² is a phenyl ring substituted with 1 to 3 substituents independently selected from R^{5a}; or a pyrazolyl or pyridinyl ring optionally substituted with up to 3 substituents independently selected from R^{5a} on carbon atom ring members and methyl on the nitrogen atom ring member.

### Embodiment 8

A compound of Embodiment 6 wherein Q² is a phenyl ring substituted with 1 to 3 substituents independently selected from R^{5a}; or a pyridinyl or pyrimidinyl ring optionally substituted with up to 3 substituents independently selected from R^{5a}.

### Embodiment 9

A compound of Formula 1 or any one of Embodiments 1 through 8 wherein Q² is a phenyl ring substituted with 1 to 3 substituents independently selected from R^{5a}.

### Embodiment 10

A compound of Embodiment 9 wherein Q² is a phenyl ring substituted with 3 substituents independently selected from R^{5a}.

### Embodiment 11

A compound of Embodiment 10 wherein Q² is a phenyl ring substituted with 2 substituents independently selected from R^{5a}.

### Embodiment 12

A compound of Formula 1 or any one of Embodiments 1 through 11 wherein Q² is a phenyl ring substituted with at least one R^{5a} substituent attached at an ortho position (relative to the connection of the Q² ring to the remainder of Formula 1).

### Embodiment 13

A compound of Formula 1 or any one of Embodiments 1 through 12 wherein when each Q¹ and Q² is a phenyl ring substituted with 1 to 3 substituents independently selected from R^{5a}, then one of the Q¹ and Q² rings is substituted with 2 or 3 substituents and the other of the Q¹ and Q² rings is substituted with 1, 2 or 3 substituents.

### Embodiment 14

A compound of Formula 1 or any one of Embodiments 1 through 13 wherein when each Q¹ and Q² is a phenyl ring substituted with 1 to 3 substituents independently selected from R^{5a}, then one of the Q¹ and Q² rings is substituted with 2 or 3 substituents and the other of the Q¹ and Q² rings is substituted with 1 or 2 substituents.

### Embodiment 15

A compound of Formula 1 or any one of Embodiments 1 through 14 wherein when each Q¹ and Q² is a phenyl ring substituted with 1 to 3 substituents independently selected from R^{5a}, then one of the Q¹ and Q² rings is substituted with 3 substituents and the other of the Q¹ and Q² rings is substituted with 2 substituents.

### Embodiment 16

A compound of Formula 1 or any one of Embodiments 1 through 15 wherein when each Q¹ and Q² is a phenyl ring substituted with 1 to 3 substituents independently selected from R^{5a}, then both of the Q¹ and Q² rings are substituted with 2 substituents.

### Embodiment 17

A compound of Formula 1 or any one of Embodiments 1 through 16 wherein when each Q¹ and Q² is a phenyl ring substituted with 1 to 3 substituents independently selected from R^{5a}, then the R^{5a} substituents are attached at the ortho and/or para positions.

### Embodiment 18

A compound of Formula 1 or any one of Embodiments 1 through 17 wherein R¹ and R² are each independently H, halogen, cyano, C₁-C₃ alkyl or cyclopropyl.

### Embodiment 19

A compound of Embodiment 18 wherein R¹ and R² are each independently H, halogen, cyano or C₁-C₃ alkyl.

### Embodiment 20

A compound of Embodiment 18 wherein R¹ and R² are each independently halogen, cyano or C₁-C₃ alkyl.

### Embodiment 21

A compound of Embodiment 18 wherein R¹ and R² are each independently H, halogen, methyl or cyclopropyl.

### Embodiment 22

A compound of Embodiment 18 wherein R¹ and R² are each independently halogen, methyl or cyclopropyl.

### Embodiment 23

A compound of Embodiment 18 wherein R¹ and R² are each independently H, Cl, Br, I or C₁-C₂ alkyl.

### Embodiment 24

A compound of Embodiment 18 wherein R¹ and R² are each independently Cl, Br, I or C₁-C₂ alkyl.

### Embodiment 25

A compound of Embodiment 18 wherein R¹ and R² each independently Cl, Br or methyl.

### Embodiment 26

A compound of Formula 1 or any one of Embodiments 1 through 25 wherein R³ is Br, Cl, F, —OR⁶ or —SC≡N.

### Embodiment 27

A compound of Embodiment 26 wherein R³ is Br, Cl, F or —OR⁶.

### Embodiment 28

A compound of Embodiment 27 wherein R³ is —OR⁶.

### Embodiment 29

A compound of Formula 1 or any one of Embodiments 1 through 25 wherein R³ is halogen.

### Embodiment 30

A compound of Embodiment 29 wherein R³ is Br, Cl or F.

### Embodiment 31

A compound of Formula 1 or any one of Embodiments 1 through 30 wherein R⁴ is H or methyl.

### Embodiment 32

A compound of Embodiment 31 wherein R⁴ is H.

### Embodiment 33

A compound of Formula 1 or any one of Embodiments 1 through 32 wherein each R^{5a} is independently halogen, cyano, C₁-C₂ alkyl, C₁-C₂ haloalkyl, cyclopropyl, C₁-C₂ alkoxy, C₁-C₂ alkylthio or -T-U—V.

### Embodiment 34

A compound of Embodiment 33 wherein each R^{5a} is independently halogen, cyano, methyl, halomethyl, cyclopropyl, methoxy, methylthio or -T-U—V.

### Embodiment 35

A compound of Embodiment 34 wherein each R^{5a} is independently halogen, cyano, methyl, halomethyl or methoxy.

### Embodiment 36

A compound of Embodiment 35 wherein each R^{5a} is independently halogen, cyano or methoxy.

### Embodiment 37

A compound of Embodiment 36 wherein each R^{5a} is independently Br, Cl, F, cyano or methoxy.

### Embodiment 38

A compound of Embodiment 37 wherein each R^{5a} is independently Br, Cl, F, or methoxy.

### Embodiment 39

A compound of Embodiment 38 wherein each R^{5a} is independently Br, Cl or F.

### Embodiment 40

A compound of Embodiment 39 wherein each R^{5a} is independently Cl or F.

### Embodiment 41

A compound of Formula 1 or any one of Embodiments 1 through 40 wherein each R^{5b} is independently cyano, C₁-C₂ alkyl, cyclopropyl or C₂-C₃ alkoxyalkyl.

### Embodiment 42

A compound of Embodiment 41 wherein each R^{5b} is methyl.

### Embodiment 43

A compound of Formula 1 or any one of Embodiments 1 through 42 wherein R⁶ is H, —CH(═O), C₁-C₃ alkyl, C₁-C₂ haloalkyl, C₂-C₃ alkoxyalkyl, C₂-C₄ cyanoalkyl, C₂-C₄ alkylcarbonyl, C₂-C₄ alkoxycarbonyl, C₂-C₄ (alkylthio)carbonyl or C₂-C₄ alkoxy(thiocarbonyl).

### Embodiment 44

A compound of Embodiment 43 wherein R⁶ is H, —CH(═O), C₁-C₃ alkyl, C₁-C₂ haloalkyl, C₂-C₃ alkoxyalkyl, C₂-C₄ cyanoalkyl, C₂-C₄ alkylcarbonyl or C₂-C₄ alkoxycarbonyl.

### Embodiment 45

A compound of Embodiment 44 wherein R⁶ is H, —CH(═O), methyl, halomethyl, cyanomethyl, methylcarbonyl or methoxycarbonyl.

### Embodiment 46

A compound of Embodiment 45 wherein R⁶ is H.

### Embodiment 47

A compound of Formula 1 or any one of Embodiments 1 through 46 wherein each T is independently O, N(R⁷) or a direct bond.

### Embodiment 48

A compound of Embodiment 47 wherein each R⁷ is independently H or methyl.

### Embodiment 49

A compound of Embodiment 47 wherein each T is independently O, NH or a direct bond.

### Embodiment 50

A compound of Formula 1 or any one of Embodiments 1 through 49 wherein each U is independently C₁-C₄ alkylene, wherein up to 1 carbon atom is selected from C(═O).

### Embodiment 51

A compound of Embodiment 50 wherein each U is independently C₁-C₃ alkylene.

### Embodiment 52

A compound of Formula 1 or any one of Embodiments 1 through 51 wherein each V is independently N(R^{8a})(R^{8b}) or OR⁹.

### Embodiment 53

A compound of Formula 1 or any one of Embodiments 1 through 52 wherein each R^{8a} and R^{8b} is independently H, C₁-C₆ alkyl or C₁-C₆ haloalkyl.

### Embodiment 54

A compound of Embodiment 53 wherein each R^{8a} and R^{8b} is independently H or methyl.

### Embodiment 55

A compound of Formula 1 or any one of Embodiments 1 through 54 wherein each R⁹ is independently H, C₁-C₆ alkyl or C₁-C₆ haloalkyl.

### Embodiment 56

A compound Embodiment 55 wherein each R⁹ is independently H, methyl or halomethyl.

Embodiments of this invention, including Embodiments 1-56 above as well as any other embodiments described herein, can be combined in any manner, and the descriptions of variables in the embodiments pertain not only to the compounds of Formula 1 but also to the starting compounds and intermediate compounds useful for preparing the compounds of Formula 1. In addition, embodiments of this invention, including Embodiments 1-56 above as well as any other embodiments described herein, and any combination thereof, pertain to the compositions and methods of the present invention.

Combinations of Embodiments 1-56 are illustrated by:

### Embodiment A1

A compound of Formula 1 wherein
Q¹ is a phenyl ring substituted with 1 to 3 substituents independently selected from R^{5a}; or a pyridinyl or pyrimidinyl ring optionally substituted with up to 3 substituents independently selected from R^{5a};
Q² is a phenyl ring substituted with 1 to 3 substituents independently selected from R^{5a}; or a pyrazolyl, pyridinyl or pyrimidinyl ring optionally substituted with up to 3 substituents independently selected from R^{5a} on carbon atom ring members and methyl on the nitrogen atom ring member;
R¹ and R² are each independently H, halogen, cyano, C₁-C₃ alkyl or cyclopropyl;
R³ is Br, Cl, F, —OR⁶ or —SC≡N;
R⁴ is H or methyl;
each R^{5a} is independently halogen, cyano, C₁-C₂ alkyl, C₁-C₂ haloalkyl, cyclopropyl, C₁-C₂ alkoxy, C₁-C₂ alkylthio or -T-U—V;
R⁶ is H, —CH(═O), C₁-C₃ alkyl, C₁-C₂ haloalkyl, C₂-C₃ alkoxyalkyl, C₂-C₄ cyanoalkyl, C₂-C₄ alkylcarbonyl, C₂-C₄ alkoxycarbonyl, C₂-C₄ (alkylthio)carbonyl or C₂-C₄ alkoxy(thiocarbonyl);
each T is independently O, NH or a direct bond;
each U is independently C₁-C₃ alkylene, wherein up to 1 carbon atom is selected from C(═O);
each V is independently N(R^{8a})(R^{8b}) or OR⁹;
each R^{8a} and R^{8b} is independently H or methyl; and
each R⁹ is independently H, methyl or halomethyl.

### Embodiment A2

A compound of Embodiment A1 wherein
Q¹ is a phenyl ring substituted with 1 to 3 substituents independently selected from R^{5a};
Q² is a phenyl ring substituted with 1 to 3 substituents independently selected from R^{5a};
R¹ and R² are each independently H, Cl, Br, I or C₁-C₂ alkyl; and
each R^{5a} is independently halogen, cyano, methyl, halomethyl, cyclopropyl, methoxy, methylthio or -T-U—V.

### Embodiment A3

A compound of Embodiment A2 wherein
R¹ and R² are each independently Cl, Br or methyl;
R³ is —OR⁶;
R⁴ is H; and
R⁶ is H, —CH(═O), C₁-C₃ alkyl, C₁-C₂ haloalkyl, C₂-C₃ alkoxyalkyl, C₂-C₄ cyanoalkyl, C₂-C₄ alkylcarbonyl or C₂-C₄ alkoxycarbonyl.

### Embodiment A4

A compound of Embodiment A3 wherein
each R^{5a} is independently Br, Cl, F, cyano or methoxy;
R⁶ is H; and
one of the Q¹ and Q² rings is substituted with 2 or 3 substituents and the other of the Q¹ and Q² rings is substituted with 1 or 2 substituents.

Specific embodiments include compounds of Formula 1 selected from the group consisting of:
2,4-dichloro-α-(2-chloro-4-fluorophenyl)-1-(2,6-difluorophenyl)-1H-imidazole-5-methanol;
2-chloro-α-(2-chloro-4-fluorophenyl)-1-(2,6-difluorophenyl)-4-methyl-1H-imidazole-5-methanol;
2-bromo-α-(2-chloro-4-fluorophenyl)-1-(2,6-difluorophenyl)-4-methyl-1H-imidazole-5-methanol;
4-bromo-2-chloro-α-(2-chloro-4-fluorophenyl)-1-(2,6-difluorophenyl)-1H-imidazole-5-methanol;
2,4-dichloro-α-(2-chloro-4-fluorophenyl)-1-(2-chloro-6-fluorophenyl)-1H-imidazole-5-methanol;
2-chloro-α-(2-chloro-4-fluorophenyl)-1-(2-chloro-6-fluorophenyl)-4-methyl-1H-imidazole-5-methanol;
2-bromo-α-(2-chloro-4-fluorophenyl)-1-(2-chloro-6-fluorophenyl)-4-methyl-1H-imidazole-5-methanol;
4-bromo-2-chloro-α-(2-chloro-4-fluorophenyl)-1-(2-chloro-6-fluorophenyl)-1H-imidazole-5-methanol;
2,4-dichloro-1-(2-chloro-4,6-difluorophenyl)-α-(2-chloro-4-fluorophenyl)-1H-imidazole-5-methanol;
2-chloro-1-(2-chloro-4,6-difluorophenyl)-α-(2-chloro-4-fluorophenyl)-4-methyl-1H-imidazole-5-methanol;
2-bromo-1-(2-chloro-4,6-difluorophenyl)-α-(2-chloro-4-fluorophenyl)-4-methyl-1H-imidazole-5-methanol;
4-bromo-2-chloro-1-(2-chloro-4,6-difluorophenyl)-α-(2-chloro-4-fluorophenyl)-1H-imidazole-5-methanol;
1-(2-bromo-4,6-difluorophenyl)-2,4-dichloro-α-(2-chloro-4-fluorophenyl)-1H-imidazole-5-methanol;
1-(2-bromo-4,6-difluorophenyl)-2-chloro-α-(2-chloro-4-fluorophenyl)-4-methyl-1H-imidazole-5-methanol;
2-bromo-1-(2-bromo-4,6-difluorophenyl)-α-(2-chloro-4-fluorophenyl)-4-methyl-1H-imidazole-5-methanol;
4-bromo-1-(2-bromo-4,6-difluorophenyl)-2-chloro-α-(2-chloro-4-fluorophenyl)-1H-imidazole-5-methanol;
2,4-dichloro-α-(2-chloro-4-fluorophenyl)-1-(2,4-difluorophenyl)-1H-imidazole-5-methanol;
2-chloro-α-(2-chloro-4-fluorophenyl)-1-(2,4-difluorophenyl)-4-methyl-1H-imidazole-5-methanol;
2-bromo-α-(2-chloro-4-fluorophenyl)-1-(2,4-difluorophenyl)-4-methyl-1H-imidazole-5-methanol;
4-bromo-2-chloro-α-(2-chloro-4-fluorophenyl)-1-(2,4-difluorophenyl)-1H-imidazole-5-methanol;
2,4-dichloro-α,1-bis(2-chloro-4-fluorophenyl)-1H-imidazole-5-methanol;
2-chloro-α,1-bis(2-chloro-4-fluorophenyl)-4-methyl-1H-imidazole-5-methanol;
2-bromo-α,1-bis(2-chloro-4-fluorophenyl)-4-methyl-1H-imidazole-5-methanol;
4-bromo-2-chloro-α,1-bis(2-chloro-4-fluorophenyl)-1H-imidazole-5-methanol;
1-(2-bromo-4-fluorophenyl)-2,4-dichloro-α-(2-chloro-4-fluorophenyl)-1H-imidazole-5-methanol;
1-(2-bromo-4-fluorophenyl)-2-chloro-α-(2-chloro-4-fluorophenyl)-4-methyl-1H-imidazole-5-methanol;
2-bromo-1-(2-bromo-4-fluorophenyl)-α-(2-chloro-4-fluorophenyl)-4-methyl-1H-imidazole-5-methanol;
4-bromo-1-(2-bromo-4-fluorophenyl)-2-chloro-α-(2-chloro-4-fluorophenyl)-1H-imidazole-5-methanol;
2-bromo-4-chloro-1-(2-chloro-4,6-difluorophenyl)-α-(2,4-difluorophenyl)-1H-imidazole-5-methanol;
2-bromo-1-(2-chloro-4,6-difluorophenyl)-α-(2,4-difluorophenyl)-4-methyl-1H-imidazole-5-methanol;
2-chloro-α-(2-chloro-4-methoxyphenyl)-1-(2,6-difluorophenyl)-4-methyl-1H-imidazole-5-methanol;
2-chloro-1-(2,6-difluorophenyl)-α-(4-methoxy-2-methylphenyl)-4-methyl-1H-imidazole-5-methanol;
2-chloro-1-(2-chloro-6-fluorophenyl)-α-(4-methoxy-2-methylphenyl)-4-methyl-1H-imidazole-5-methanol;
4-bromo-2-chloro-α-(2-chloro-4-methoxyphenyl)-1-(2,6-difluorophenyl)-1H-imidazole-5-methanol;
4-bromo-1-(2-chloro-6-fluorophenyl)-α-(4-methoxy-2-methylphenyl)-2-methyl-1H-imidazole-5-methanol;
4-chloro-1-(2-chloro-4,6-difluorophenyl)-α-(2-chloro-4-methoxyphenyl)-2-methyl-1H-imidazole-5-methanol;
4-chloro-α-(2-chloro-4-methoxyphenyl)-1-(2,6-difluorophenyl)-2-methyl-1H-imidazole-5-methanol;
2-chloro-1-(2-chloro-4,6-difluorophenyl)-α-(2,4-difluorophenyl)-4-methyl-1H-imidazole-5-methanol;
2-chloro-1-(2-chloro-4,6-diflorophenyl)-α-(2-chloro-4-fluorophenyl)-4-methyl-1H-imidazole-5-methanol;
2-chloro-1-(2-chloro-4,6-difluorophenyl)-α-(4-fluoro-2-methylphenyl)-4-methyl-1H-imidazole-5-methanol;
2-chloro-1-(2-chloro-4-fluorophenyl)-α-(2-chloro-4-methoxyphenyl)-4-methyl-1H-imidazole-5-methanol;
2-chloro-1-(2-chloro-4-fluorophenyl)-α-(4-methoxy-2-methylphenyl)-4-methyl-1H-imidazole-5-methanol;
4-bromo-2-chloro-α,1-bis(2-chloro-4-fluorophenyl)-1H-imidazole-5-methanol;
2,4-dichloro-1-(2-chloro-4,6-difluorophenyl)-α-(2,4-difluorophenyl)-1H-imidazole-5-methanol;
2-chloro-1-(2-chloro-6-fluorophenyl)-α-(2,4,-difluorophenyl)-4-methyl-1H-imidazole-5-methanol;
1-(2-bromo-6-fluorophenyl)-2-chloro-α-(2,4-difluorophenyl)-4-methyl-1H-imidazole-5-methanol and
1-(2-bromo-6-fluorophenyl)-2-chloro-α-(4-methoxy-2-methylphenyl)-4-methyl-1H-imidazole-5-methanol.

Of note are compounds of Formula 1 (including all stereoisomers), N-oxides, and salts thereof (including but not limited to Embodiments 1-56 above) wherein R¹ and R² are each independently H, halogen, cyano, nitro, C₁-C₃ alkyl, C₂-C₃ alkenyl, C₂-C₃ alkynyl, C₁-C₃ haloalkyl, C₂-C₃ haloalkenyl, cyclopropyl, halocyclopropyl, C₁-C₃ hydroxyalkyl, C₂-C₃ cyanoalkyl, C₁-C₃ alkoxy, C₁-C₃ haloalkoxy, C₁-C₃ alkylthio or C₁-C₃ haloalkylthio.

Of further note are compounds of Formula 1 (including all stereoisomers), N-oxides, and salts thereof (including but not limited to Embodiments 1-56 above) wherein R³ is halogen or —OR⁶.

Additionally, of note are compounds of Formula 1 (including all stereoisomers), N-oxides, and salts thereof (including but not limited to Embodiments 1-56 above) wherein each R^{5a} is independently halogen, cyano, hydroxy, nitro, C₁-C₃ alkyl, C₂-C₃ alkenyl, C₂-C₃ alkynyl, C₁-C₃ haloalkyl, C₂-C₃ haloalkenyl, cyclopropyl, halocyclopropyl, C₂-C₃ cyanoalkyl, C₁-C₃ alkylthio, C₁-C₃ haloalkylthio, C₁-C₃ alkylsulfinyl, C₁-C₃ haloalkylsulfinyl, C₁-C₃ alkylsulfonyl, C₁-C₃ haloalkylsulfonyl, C₁-C₃ alkoxy, C₁-C₃ haloalkoxy, C₂-C₃ alkylcarbonyl, C₁-C₃ alkylamino, C₂-C₄ dialkylamino, C₂-C₃ alkylcarbonylamino, C₃-C₆ trialkylsilyl, —NHCH(═O), —C(═S)NH₂, —SC≡N or -T-U—V.

This invention provides a fungicidal composition comprising a compound of Formula 1 (including all geometric and stereoisomers, N-oxides, and salts thereof), and at least one other fungicide. Of note as embodiments of such compositions are compositions comprising a compound corresponding to any of the compound embodiments described above.

This invention provides a fungicidal composition comprising a fungicidally effective amount of a compound of Formula 1 (including all geometric and stereoisomers, N-oxides, and salts thereof), and at least one additional component selected from the group consisting of surfactants, solid diluents and liquid diluents. Of note as embodiments of such compositions are compositions comprising a compound corresponding to any of the compound embodiments described above.

This invention provides a method for controlling plant diseases caused by fungal plant pathogens comprising applying to the plant or portion thereof, or to the plant seed, a fungicidally effective amount of a compound of Formula 1 (including all geometric and stereoisomers, N-oxides, and salts thereof). Of note as embodiment of such methods are methods comprising applying a fungicidally effective amount of a compound corresponding to any of the compound embodiments describe above. Of particular note are embodiments where the compounds are applied as compositions of this invention.

One or more of the following methods and variations as described in Schemes 1-14 can be used to prepare the compounds of Formula 1. The definitions of Q¹, Q2, R¹, R², R³ and R⁴ in the compounds of Formulae 1-20 below are as defined above in the Summary of the Invention unless otherwise noted. Compounds of Formula 1a-1c are various subsets of Formula 1, and all substituents for Formula 1a-1c are as defined above for Formula 1 unless otherwise noted.

As shown in Scheme 1, compounds of Formula 1a (i.e. Formula 1 wherein R³ is —OR⁶ and R⁶ is H) can be prepared by contacting keto compounds of Formula 2 with organometallic reagents of formula Q¹-M¹ wherein M¹ is MgX¹, Li or ZnX¹ and X¹ is Cl, Br or I. Typically the reaction is carried out in a suitable solvent such as tetrahydrofuran, diethyl ether or toluene at a temperature between about −78 to 20° C. Reactions of this type can be found in the chemistry literature; see, for example, Koswatta et al., Organic Letters 2008, 10(21), 5055-5058 and Koswatta et al., Synthesis 2009, (17), 2970-2982. Also, the method of Scheme 1 is illustrated in present Example 1, Step E and Example 6, Step C.

Compounds of formula Q¹-M¹ are commercially available and can be prepared by methods well-known to one skilled in the art.

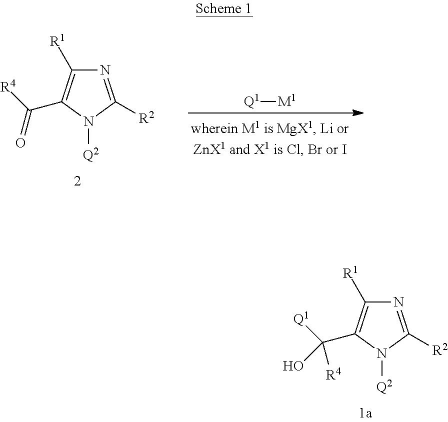

As shown in Scheme 2, compounds of Formula 1a (i.e. Formula 1 wherein R³ is —OR⁶ and R⁶ is H) can also be prepared by a method analogous to Scheme 1 wherein the substituents Q¹ and R⁴ are interchanged. In Method A ketones of Formula 3 containing Q¹ are reacted with organometallic reagents of formula R⁴-M¹ using reaction conditions as described in Scheme 1 to provide compounds of Formula 1a wherein R⁴ is alkyl. Present Example 3, Step F illustrates this method using methyllithium. In Method B compounds of Formula 3 are contacted with hydride-containing reducing agents such as sodium borohydride, diisobutylaluminum hydride or lithium aluminum hydride in a solvent such as methanol, ethanol, tetrahydrofuran or diethyl ether at a temperature between about −20 to 20° C. to provide compounds of Formula 1a wherein R⁴ is H. Example 2, Step F illustrates this method using sodium borohydride.

Other reduction techniques known to those skilled in the art may also be employed to obtain compounds of Formula 1a wherein R⁴ is H. For example, ketones of Formula 3 can be reduced by catalytic hydrogenation as shown in Scheme 2, Method C. Typical reaction conditions involve exposing a compound of Formula 3 to hydrogen gas at a pressure of about 70 to 700 kPa, in the presence of a metal catalyst such as palladium or ruthenium supported on an inert carrier such as activated carbon, in a solvent such as ethanol at about 20° C. This type of reduction is well-known; see, for example, Catalytic Hydrogenation, L. Cerveny, Ed., Elsevier Science, Amsterdam, 1986, Tetrahedron: Asymmetry 2009, 20(5), 605-609 and Tetrahedron Letters 1995, 36(50), 9153-9156. One skilled in the art will recognize that certain other functionalities that may be present in compounds of Formula 3 can also be reduced under catalytic hydrogenation conditions, thus requiring a suitable choice of catalyst and conditions. In some cases the presence of a chiral diamine ligand having at least one N—H bond results in higher chemoselectivity of the desired compound (i.e. the carbonyl moiety is selectively reduced over certain other functionalities that may be present in compounds of Formula 3). For conditions and variations of this reaction see, for example, Praetorius et al., Organometallics 2010, 29(3), 554-561.

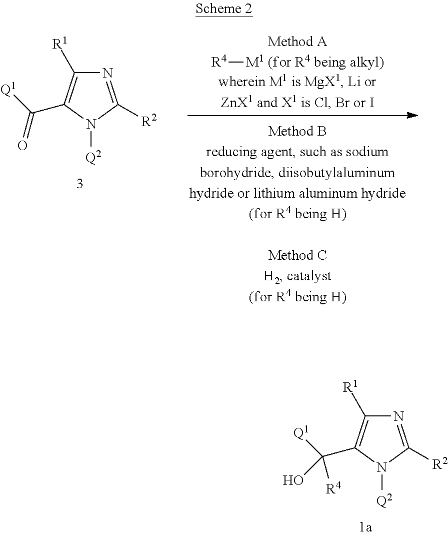

As shown in Scheme 3, Compounds of Formula 1a (i.e. Formula 1 wherein R³ is —OR⁶ and R⁶ is H) can be converted to the compounds of Formula 1b (i.e. Formula 1 wherein R³ is halogen) using a variety of conditions published in the chemical literature. For example, treatment of a compound of Formula 1a with a fluorinating agent (e.g., bis(2-methoxyethyl)aminosulfur (Deoxo-Fluor®), diethylaminosulfur trifluoride (DAST), HF-pyridine (Olah's reagent) or sulfur tetrafluoride) provides compounds of Formula 1b wherein R³ is F. For reaction conditions see C. J. Wang, Organic Reactions 2005, Vol. 34 (Wiley, New York, 1951) Chapter 2, pp. 319-321. Compounds of Formula 1b wherein R³ is Br can be prepared by treating the corresponding compound of Formula 1a with hydrobromic acid in a solvent such as glacial acetic acid using the method described by Beukers et al., Journal of Medicinal Chemistry 2004, 47(15), 3707-3709. Compounds of Formula 1b wherein R³ is Cl can be prepared by treating the corresponding compound of Formula 1a with thionyl chloride or phosphorus pentachloride in the presence of a base such as triethylamine or pyridine in a solvent such as dichloromethane or pyridine at 25-110° C. Compounds of Formula 1b wherein R³ is I can be prepared by reacting the corresponding compound of Formula 1a with sodium iodide or potassium iodide in the presence of BF₃.Et₂O and an ether solvent such as 1,4-dioxane or with hydroiodic acid in a solvent such as acetonitrile at 25-70° C. according to general methods described in Tetrahedron Letters 2001, 42, 951-953 and Journal of the American Chemical Society 1965, 87, 539-42.

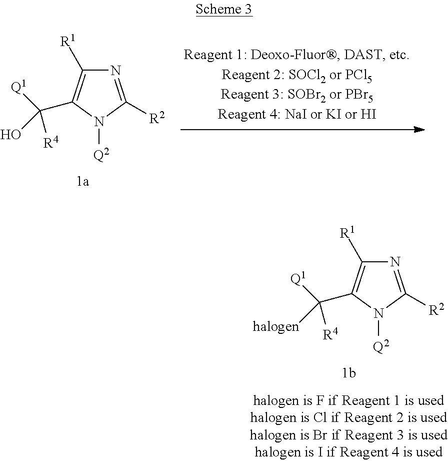

As shown in Scheme 4, compounds of Formula 1c (i.e. Formula 1 wherein R¹ is halogen) can be prepared by treating compounds of Formula 1 wherein R¹ is H with the corresponding N-halosuccinimide in the presence of a suitable solvent such as N,N-dimethylformamide or acetonitrile at 20 to 80° C. for a time period of about 30 minutes to 20 h, according to general procedures known in the art such as described in Tetrahedron Letters 2009, 50, 5762-5764. Example 5 and Example 6, Step D illustrate the method of Scheme 4 using NBS.

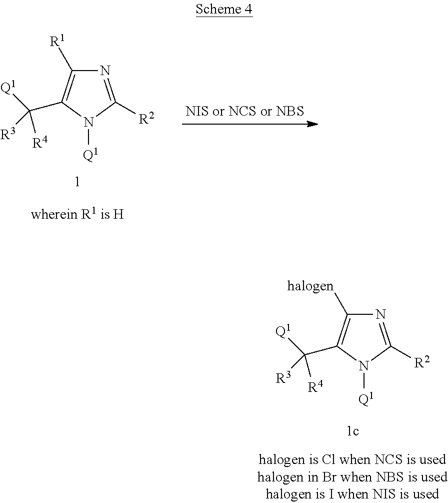

As shown in Scheme 5, to introduce a fluoro at the 4-position of the imidazole ring, compounds of Formula 1c wherein the halogen is Cl are treated with potassium fluoride or cesium fluoride in the presence of a solvent such as dimethyl sulfoxide or N,N-dimethylformamide at 0-25° C. for time periods of 30 minutes to 4 h, using procedures such as described in Zhurnal Organicheskoi Khimii 1983, 19, 2164-73.

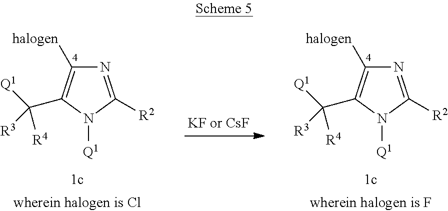

In the method of Scheme 4 halogenation typically occurs preferentially at the 4-position of the imidazole ring to provide a compound of Formula 1c (i.e. Formula 1 wherein R¹ is halogen). To obtain compounds of Formula 1 wherein R¹ and R² are both halogen, Compounds of Formula 1c can be treated with a second equivalent of the same halogenating reagent (for R¹ and R² being the same halogen) or a different halogenating reagent (for R¹ and R² being different halogens) using appropriate variations of the methods of Schemes 4 and 5. For an example illustrating the method of preparing a compound of Formula 1 wherein R¹ and R² are different halogens see Example 7.

As is shown in Scheme 6, intermediate compounds of Formula 2 wherein R⁴ is alkyl can be prepared by contacting an organometallic reagent of formula R⁴-M² with an amide of Formula 4. In this method compounds of formula R⁴-M² are Grignard reagents (i.e. M² is MgX² and X² is Br or Cl, for example, methylmagnesium chloride or bromide) or organolithium reagents (i.e. M² is Li, for example, methyllithium or tert-butyllithium). Typically the reaction is conducted in a suitable solvent such as diethyl ether, tetrahydrofuran or toluene at a temperature between about −78 to 20° C. The compounds of Formula 2 can be isolated by quenching the reaction mixture with aqueous acid, extracting with an organic solvent and concentrating.

Compounds of Formula 2 wherein R⁴ is H can be prepared by reduction of compounds of Formula 4 with a metal hydride reducing agent such as diisobutylaluminum hydride, as shown in Scheme 6.

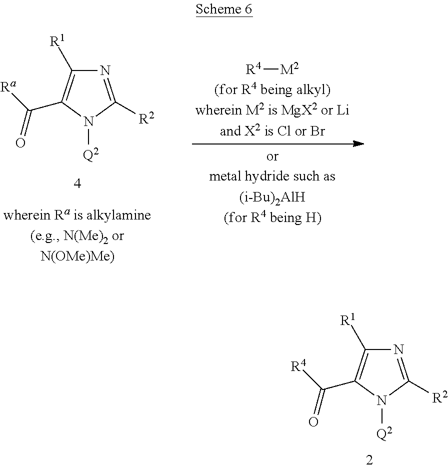

Amides of Formula 4 can be prepared by methods known in the art. For example, as shown in Scheme 7, compounds of Formula 4 wherein R^{a} is N(OMe)Me can be synthesized by conversion of a carboxylic acid of Formula 5 to the corresponding acid chloride, which can be isolated or formed in situ, as shown in Scheme 7. Treatment of the acid chloride with N,O-dimethylhydroxylamine hydrochloride provides Formula 4 wherein R^{a} is N(OMe)Me. Reactions of this type are well-known and published in the chemistry literature (e.g., publications relating to Weinreb amide reactions). For conditions and variations see the following references and references cited therein: PCT Patent Publication WO 2005/086836, De Luca et al., Journal of Organic Chemistry 2001, 66, 2534-2537 and Weinreb et al., Tetrahedron Letters, 1981, Vol. 22, No. 39, 3815-3818. Also, present Example 3, Step D lustrates the method of Scheme 7.

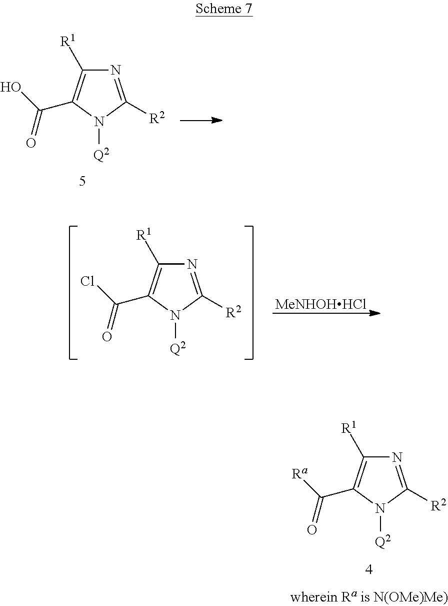

Compounds of Formula 5 can be prepared as shown in Scheme 8. In this method a compound Formula 6 is first treated with a base in an appropriate solvent such as tetrahydrofuran, diethyl ether or toluene at temperatures ranging from about −78° C. to ambient temperature. Useful bases for this reaction include lithium salts or magnesium halide salts of amine bases such as diisopropylamine or 2,2,6,6-tetramethylpiperidine. Subsequent treatment of the resulting anion (generated in situ) with an electrophile adds an R² group to the imidazole ring to provide a compound of Formula 6a. For halogenation, the electrophile can be a halogen derivative such as N-chlorosuccinimide (NCS), N-bromosuccinimide (NBS), N-iodosuccinimide (NIS), hexachloroethane, 1,2-dibromotetrachloroethane, carbon tetrabromide, hexachloroethane or a fluorinating reagent such as Accufluor® (e.g., N-fluorobis(phenylsulfonyl)amine). For alkylation, the electrophile can be an alkylating agent of the formula R²-Lg (wherein Lg is a leaving group such as Cl, Br, I or a sulfonate, for example, p-toluenesulfonate, methanesulfonate or trifluoromethanesulfonate) where R² is alkyl, alkylthio, haloalkyl, alkenyl, haloalkenyl, alkynyl, and the like. Alternatively, symmetrical electrophiles such as dialkyldisufides can be used where R² is alkylthio. As referred to herein, the terms “alkylation” and “alkylating agent” are not limited to R² being an alkyl group. For related reference see Almansa et al., Journal of Medicinal Chemistry 2003, 46, 3463-3475 Tetrahedron Letters 1994, 35(21), 3465-8 and Journal of Organic Chemistry 2001, 66(15), 5163-5173. Also, Example 3, Step B illustrates the preparation of a compound of Formula 6a using the method of Scheme 8. The resulting ester of Formula 6a can be converted to the carboxylic acid of Formula 5 using a variety of methods reported in the chemical literature, including nucleophilic cleavage under anhydrous conditions or hydrolysis involving the use of either acids or bases (see T. W. Greene and P. G. M. Wuts, Protective Groups in Organic Synthesis, 2nd ed., John Wiley & Sons, Inc., New York, 1991, pp. 224-269 for a review of methods). Base-catalyzed hydrolytic methods are preferred to prepare the carboxylic acids of Formula 5 from the corresponding esters. Suitable bases include alkali metal (such as lithium, sodium, or potassium) hydroxides. For example, the esters can be dissolved in a mixture of water and alcohol such as methanol. Upon treatment with sodium hydroxide or potassium hydroxide, the ester saponifies to provide the sodium or potassium salt of the carboxylic acid. Acidification with a strong acid, such as hydrochloric acid or sulfuric acid, gives the carboxylic acid. Example 3, Step C and PCT Publication WO 2003/016283 provide examples illustrating the base-catalyzed hydrolysis method for the conversion of an ester to an acid.

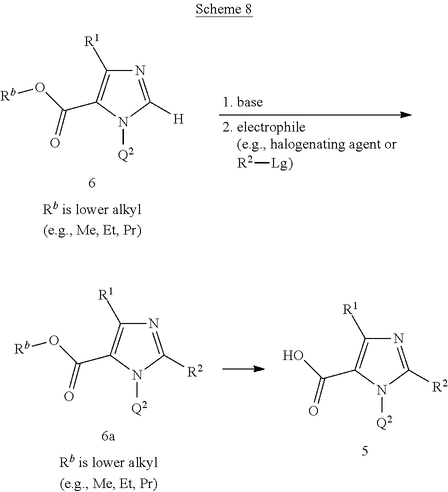

A method analogous to Scheme 8 can also be used to prepare compounds of Formula 4 wherein R² is halogen, alkyl, alkylthio, haloalkyl, alkenyl, haloalkenyl, alkynyl, and the like from the corresponding compounds of Formula 4 wherein R² is H.

In an alternative method, compounds of Formula 2 wherein R⁴ is H can be prepared by oxidation of alcohols of Formula 7 to the corresponding aldehydes as shown in Scheme 9. The oxidation reaction can be performed by a variety of means, such as by treatment of the alcohols of Formula 7 with manganese dioxide, Dess-Martin periodinane, pyridinium chlorochromate or pyridinium dichromate. The method of Scheme 9 is illustrated in Example 1, Step D and Example 6, Step B.

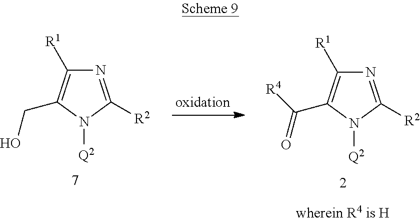

As shown in Scheme 10, compounds of Formula 2 wherein R¹ and R⁴ are H and R² is alkyl, haloalkyl, and the like, can also be prepared by condensation of an aniline of Formula 8 with a nitrile of Formula 9 in the presence of hydrogen chloride gas to make an amidine 10. Reaction of a compound of Formula 10 with 2-halomalonaldehyde 11 (i.e. 2-chloromalonaldehyde or 2-bromomalonaldehyde) in the presence of acetic acid and triethylamine catalysts provides compounds of Formula 2. For references see, for example, Ferreira et al., European Journal of Medicinal Chemistry 2007, 42(11-12), 1388-1395 and references therein. Also, present Example 4, Steps A and B illustrates the method of Scheme 10.

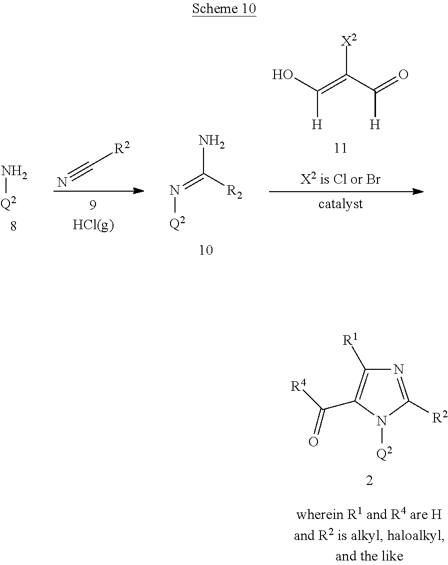

The anilines of Formula 8 and nitriles of Formula 9 are commercially available and can be prepared by methods well-known in the art. The halomalonaldehydes of Formula 11 are commercially available and can be prepared by methods known in the art, such as in described by Trofimenko, Journal of Organic Chemistry 1963, 28, 3243-3245.

Intermediate compounds of Formula 3 can be prepared using a method analogous to Scheme 6, where an aryl organometallic reagent of formula Q¹-M² is reacted with a compound of Formula 4 to provide a compound of Formula 3, as shown in Scheme 11. Example 3, Step E illustrates the method of Scheme 11.

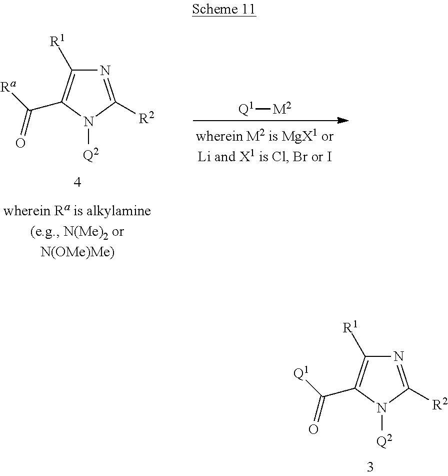

Alternatively, as shown in Scheme 12, compounds of Formula 3 can be prepared by reaction of an acid chloride of Formula 12 with a compound of formula Q¹-H using Friedel-Crafts condensation techniques. Typically the reaction is run in the presence of a Lewis acid (such as aluminum chloride or tin tetrachloride) and a solvent such as dichloromethane, 1,2-dichloroethane, tetrachloroethane, nitrobenzene or 1,2-dichlorobenzene, at a temperature between about −10 to 220° C. Friedel-Crafts reactions are documented in a variety of published references including Lutjens et al., Journal of Medicinal Chemistry 2003, 46(10), 1870-1877, PCT Patent Publication WO 2005/037758 and J. March, Advanced Organic Chemistry, McGraw-Hill, New York, p 490 and references cited within. The method of Scheme 12 is also illustrated in Step E of Example 2.

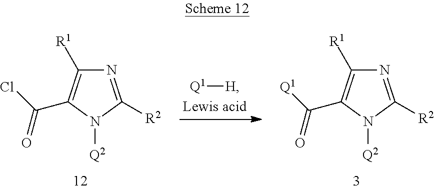

As shown in Scheme 13, intermediate compounds of Formula 7 can be obtained by reduction of an acid or ester of Formula 13. Useful reducing agents for the method of Scheme 13 include, for example, borane complexes, lithium aluminum hydride, sodium borohydride or diisobutylaluminum hydride. The method of Scheme 13 is illustrated in Example 1, Step C and Example 6, Step A.

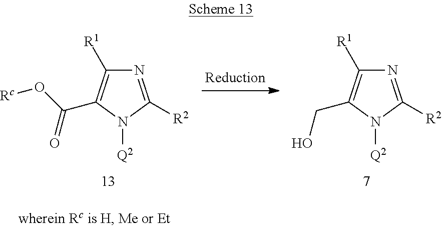

As shown in Scheme 14, compounds of Formula 13 can be prepared by treatment of an aniline of Formula 14 with a glyoxylate of Formula 15. Depending on the reactions conditions (e.g., reaction temperature and solvent) the intermediate of Formula 16 or Formula 17 is formed. Both compounds Formulae 16 and 17 undergo under cyclization when treated with a p-toluenesulfonylmethyl isocyanide of Formula 18 or benzotriazol-1-ylmethyl isocyanide of Formula 19 in the presence of a suitable base such as potassium carbonate, potassium tert-butoxide, sodium hydroxide, sodium hydride, tert-butylamine or 1,8-diazabicyclo[5.4.0]undec-7-ene (DBU) in an appropriate solvent such as methanol, dioxane, tetrahydrofuran, dimethylsulfoxide, N,N-dimethylformamide or dimethoxyethane, at temperatures ranging from about 0 to 150° C. For representative procedures see Chen et al., Tetrahedron Letters 2000, 41(29), 5453-5456, Almansa et al., Journal of Medicinal Chemistry 2003, 46(16), 3463-3475 and Katritzky et al., Heterocycles 1997, 44, 67-70. Also, the method of Scheme 14 is illustrated in Example 1, Step A, Example 2, Step A, and Example 3, Step A.

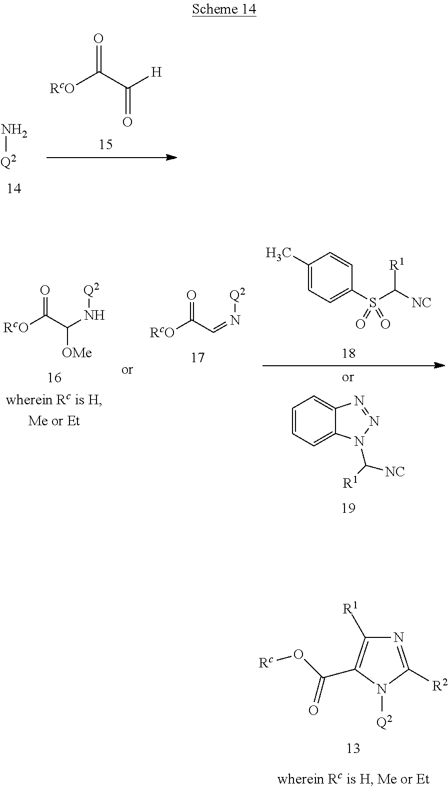

Compounds of Formula 18 are commercial available and can be prepared from unsubstituted p-toluenesulfonylmethyl isocyanide (i.e. R¹ is H) under phase-transfer conditions using methods reported in the chemical literature; see, for example, Leusen et al., Tetrahedron Letters 1975, 40, 3487-3488.

The substituted benzotriazol-1-ylmethyl isocyanides of Formula 19 can be prepared by contacting benzotriazol-1-yl-methyl isocyanide with a compound of formula R¹X³ (wherein X³ is halogen) in the presence of a base such as potassium carbonate, sodium hydride or potassium tert-butoxide. For typical reaction conditions see Katritzky et al., Heterocycles 1997, 44, 67-70. One skilled in the art will recognize other methods for preparing compounds of Formula 19, for example, the method described by Katritzky et al., Journal of the Chemical Society, Perkin Transactions 1 1990, (7), 1847-1851.

Numerous other methods for preparation of imidazoles and functionalization of imidazoles at the 2- and 4-positions exist in the art and are well-known to one skilled in the art. For representative procedures see Journal of the Chemical Society, Perkin Transactions 1: Organic and Bioorganic Chemistry 1975 (3), 275-7; Chemische Berichte 1976, 109(5), 1625-37; Synthesis 1988, (10), 767-71; Journal of Medicinal Chemistry 2003, 46(16), 3463-3475; and Russian Journal of Organic Chemistry 2009, 45(8), 1210-1213; alao World Patent Publications: WO 2009/137651, WO 2009/127615 and WO 2009/053102.

It is recognized by one skilled in the art that various functional groups can be converted into others to provide different compounds of Formula 1. Conversion of compounds of Formula 1 wherein R³ is OH to corresponding esters, carbonates and ethers is well-known to one skilled in the art.

Compounds of Formula 1 or intermediates for their preparation may contain aromatic nitro groups, which can be reduced to amino groups, and then be converted via reactions well-known in the art such as the Sandmeyer reaction, to various halides or alkylsulfides, providing other compounds of Formula 1. By similar known reactions, aromatic amines (anilines) can be converted via diazonium salts to phenols, which can then be alkylated to prepare compounds of Formula 1 with alkoxy substituents. Likewise, aromatic halides such as bromides or iodides prepared via the Sandmeyer reaction can react with alcohols under copper-catalyzed conditions, such as the Ullmann reaction or known modifications thereof, to provide compounds of Formula 1 that contain alkoxy substituents. Additionally, some halogen groups, such as fluorine or chlorine, can be displaced with alcohols under basic conditions to provide compounds of Formula 1 containing the corresponding alkoxy substituents. The resultant alkoxy compounds can themselves be used in further reactions to prepare compounds of Formula 1 wherein R^{5a} is -T-U—V (see, for example, PCT Publication WO 2007/149448). Compounds of Formula 1 or precursors thereof in which R¹ or R² are halide, preferably bromide or iodide, are particularly useful intermediates for transition metal-catalyzed cross-coupling reactions to prepare compounds of Formula 1. These types of reactions are well documented in the literature; see, for example, Tsuji in Transition Metal Reagents and Catalysts: Innovations in Organic Synthesis, John Wiley and Sons, Chichester, 2002; Tsuji in Palladium in Organic Synthesis, Springer, 2005; and Miyaura and Buchwald in Cross Coupling Reactions: A Practical Guide, 2002; and references cited therein.

One skilled in the art will recognize that sulfide groups can be oxidized to the corresponding sulfoxides or sulfones by conditions well-known in the art.

It is recognized that some reagents and reaction conditions described above for preparing compounds of Formula 1 may not be compatible with certain functionalities present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, Greene, T. W.; Wuts, P. G. M. Protective Groups in Organic Synthesis, 2nd ed.; Wiley: New York, 1991). One skilled in the art will recognize that, in some cases, after the introduction of a given reagent as it is depicted in any individual scheme, it may be necessary to perform additional routine synthetic steps not described in detail to complete the synthesis of compounds of Formula 1. One skilled in the art will also recognize that it may be necessary to perform a combination of the steps illustrated in the above schemes in an order other than that implied by the particular sequence presented to prepare the compounds of Formula 1. One skilled in the art will also recognize that compounds of Formula 1 and the intermediates described herein can be subjected to various electrophilic, nucleophilic, radical, organometallic, oxidation, and reduction reactions to add substituents or modify existing substituents.

Without further elaboration, it is believed that one skilled in the art using the preceding description can utilize the present invention to its fullest extent. The following Synthesis Examples are, therefore, to be construed as merely illustrative, and not limiting of the disclosure in any way whatsoever. Steps in the following Synthesis Examples illustrate a procedure for each step in an overall synthetic transformation, and the starting material for each step may not have necessarily been prepared by a particular preparative run whose procedure is described in other Examples or Steps. Percentages are by weight except for chromatographic solvent mixtures or where otherwise indicated. Parts and percentages for chromatographic solvent mixtures are by volume unless otherwise indicated. ¹H NMR spectra are reported in ppm downfield from tetramethylsilane in CDCl₃ unless otherwise noted; “s” means singlet, “d” means doublet, “t” means triplet, “q” means quartet, “m” means multiplet, “br s” means broad singlet, “dt” means doublet of triplets.

### Example 1

### Preparation of 4-chloro-α-(2-chloro-4-fluorophenyl)-1-(2,6-difluorophenyl)-1H-imidazole-5-methanol (Compound 1)

### Step A: Preparation of ethyl 1-(2,6-difluorophenyl)-1H-imidazole-5-carboxylate

To a mixture of 2,6-difluorobenzeneamine (4.32 g, 33.5 mmol) in methanol (100 mL) was added ethyl glyoxylate (50% solution in toluene, 33 mL). The reaction mixture was heated at 60° C. for 16 h, and then concentrated under reduced pressure. The resulting material was diluted with toluene and concentrated under reduced pressure (2×100 mL) to provide a yellow oil (12.55 g). To a mixture of the yellow oil in methanol (100 mL) was added 1-[(isocyanomethyl)sulfonyl]-4-methylbenzene (also known as p-toluenesulfonyl-methylisonitrile) (8.6 g, 44 mmol) and powdered potassium carbonate (12 g, 87 mmol). The reaction mixture was heated at 50 to 53° C. for 3.5 h, and then concentrated under reduced pressure. The resulting material was diluted with ethyl acetate (100 mL) and filtered through a pad of silica gel on a sintered glass frit funnel. The filtrate was concentrated under reduced pressure, diluted with hexanes-ethyl acetate (2:1, 20 mL), warmed to about 45° C. and allowed to stand. After 3 days, the hexanes-ethyl acetate mixture was filtered to provide the title compound as a white solid (2.04 g). The filtrate was concentrated under reduced pressure and the resulting material purified by silica gel column chromatography (33 to 40% gradient of ethyl acetate in hexanes as eluant) to provide more of the title compound as a yellow solid (1.18 g).

¹H NMR (CDCl₃): δ 7.90 (s, 1H), 7.66 (s, 1H), 7.45 (m, 1H), 7.08 (m, 2H), 4.23 (q, 2H), 1.25 (t, 3H).

### Step B: Preparation of ethyl 4-chloro-1-(2,6-difluorophenyl)-1H-imidazole-5-carboxylate

To a mixture of ethyl 1-(2,6-difluorophenyl)-1H-imidazole-5-carboxylate (i.e. the product of Step A) (0.50 g, 2 mmol) in acetonitrile (4 mL) was added N-chlorosuccinimide (0.29 g, 2.2 mmol) and the mixture was heated at 80° C. After 17 h, more N-chlorosuccinimide (0.10 g, 0.7 mmol) was added to the reaction mixture and heating was continued at 80° C. After 4 h, more N-chlorosuccinimide (0.10 g, 0.7 mmol) was added to the reaction mixture and heating was continued at 80° C. for 20 h. The reaction mixture was allowed to cool to ambient temperature (about 20° C.) and partitioned between water and ethyl acetate (1:1, 40 mL). The organic phase was separated, dried over magnesium sulfate, filtered and concentrated under reduced pressure. The resulting material was purified by silica gel column chromatography (20% ethyl acetate in hexanes as eluant) to provided the title compound (0.29 g).

¹H NMR (CDCl₃): δ 7.80 (1, 1H), 7.51 (m, 1H), 7.10 (m, 2H), 4.21 (q, 2H), 1.23 (t, 3H).

### Step C: Preparation of 4-chloro-1-(2,6-difluorophenyl)-1H-imidazole-5-methanol

To a mixture of ethyl 4-chloro-1-(2,6-difluorophenyl)-1H-imidazole-5-carboxylate (i.e. the product of Step B) (0.28 g, 0.98 mmol) in diethyl ether (10 mL) at about 0° C. was added lithium aluminum hydride (1.0 M solution in ether, 1.0 mL) dropwise. After 1 h, water (40 μL) was added to the reaction mixture, followed by sodium hydroxide (15% aqueous solution, 40 μL) and water (110 μL). After about 5 minutes, the reaction mixture was filtered through a pad of Celite® (diatomaceous filter aid) on a sintered glass frit funnel, and rinsed with diethyl ether (10 mL) and ethyl acetate (10 mL). The filtrate was concentrated under reduced pressure to provide the title compound as a white solid (0.196 g).

¹H NMR (CDCl₃): δ 7.53 (m, 1H), 7.13 (m, 2H), 7.08 (s, 1H), 4.44 (d, 2H), 1.6 (br s, 1H, OH).

### Step D: Preparation of 4-chloro-1-(2,6-difluorophenyl)-1H-imidazole-5-carboxaldehyde

To a mixture of 4-chloro-1-(2,6-difluorophenyl)-1H-imidazole-5-methanol (i.e. the product of Step C) (0.19 g, 0.78 mmol) in dichloromethane (7 mL) was added manganese(IV) oxide (0.60 g), the mixture was heated at reflux for 2 h, allowed to cool to ambient temperature (about 20° C.) and filtered through pad of Celite® (diatomaceous filter aid) on a sintered glass frit funnel, rinsing with dichloromethane (15 mL). The filtrate was concentrated under reduced pressure to provide the title compound.

AP⁺ (M+1) 243

### Step E: Preparation of 4-chloro-α-(2-chloro-4-fluorophenyl)-1-(2,6-difluorophenyl)-1H-imidazole-5-methanol

To a mixture of 1-bromo-2-chloro-4-fluorobenzene (0.12 mL, 0.99 mmol) in tetrahydrofuran (5 mL) at about −78° C. was added dropwise over 5 minutes n-butyllithium (2.5 M in hexanes, 0.37 mL, 0.94 mmol) while maintaining the temperature of the reaction mixture below −65° C. After the addition was complete, 4-chloro-1-(2,6-difluorophenyl)-1H-imidazole-5-carboxaldehyde (i.e. the product of Step D) in tetrahydrofuran (2 mL) was added dropwise to the reaction mixture while maintaining the reaction mixture at about −62 to −65° C. After 20 minutes, saturated aqueous ammonium chloride solution (5 mL) was added in one portion to the reaction mixture, the mixture was allowed to warm to ambient temperature (about 20° C.), and then water (1 mL) was added. The resulting mixture was poured onto a solid phase extraction tube (Varian Chem Elute®, prepacked with diatomaceous) and eluted with ethyl acetate (50 mL). The ethyl acetate eluant was concentrated under reduced pressure and the resulting material was triturated with ethyl acetate-hexanes to provide a solid. The solid was recrystallized from ethyl acetate-hexanes to provide the title compound, a compound of the present invention, as a solid (0.080 g).

¹H NMR (DMSO-d₆): δ 7.71 (m, 1H), 7.45-7.35 (m, 4H), 7.20 (m, 1H), 6.68 (m, 1H), 6.24 (br s, 1H), 5.71 (s, 1H).

### Example 2

### Preparation of 4-chloro-1-(2-chloro-4-fluorophenyl)-α-(2,4-difluorophenyl)-1H-imidazole-5-methanol (Compound 2)

### Step A: Preparation of ethyl 1-(2-chloro-4-fluorophenyl)-1H-imidazole-5-carboxylate

To a mixture of 2-chloro-4-fluorobenzenamine (10 g, 69 mmol) in ethanol (50 mL) was added ethyl glyoxylate (50% solution in toluene, 14 g). The reaction mixture was heated at 60° C. for 16 h, and then concentrated under reduced pressure. The resulting material was diluted with toluene and concentrated under reduced pressure (2×150 mL) to provide a yellow oil (14 g). To a mixture of the yellow oil in ethanol (150 mL) was added 1-[(isocyanomethyl)sulfonyl]-4-methylbenzene (15.4 g, 79 mmol) and powdered potassium carbonate (21.9 g, 159 mmol). The reaction mixture was heated at 70° C. for 12 h, and then concentrated under reduced pressure. The resulting material was partitioned between ethyl acetate and water, separated, and the aqueous layer was extracted with ethyl acetate (3×200 mL). The combined organic layers were dried over magnesium sulfate, filtered and concentrated under reduced pressure. The resulting material was purified by silica gel column chromatography (20% ethyl acetate in hexanes as eluant) to provide the title compound as an oil (4.5 g).

¹H NMR (CDCl₃): δ 7.86 (m, 1H), 7.59 (m, 1H), 7.36-7.33 (m, 1H), 7.3-7.28 (m, 1H), 7.13-7.09 (m, 1H), 4.20 (m, 2H), 1.25 (m, 3H).

### Step B: Preparation of ethyl 4-chloro-1-(2-chloro-4-fluorophenyl)-1H-imidazole-5-carboxylate

To a mixture of ethyl 1-(2-chloro-4-fluorophenyl)-1H-imidazole-5-carboxylate (i.e. the product of Step A) (2.5 g, 9.3 mmol) in carbon tetrachloride (25 mL) was added N-chlorosuccinimide (2.49 g, 18.6 mmol) and 2,2′-(1,2-diazenediyl)bis[2-methyl-propanenitrile (AIBN) (76 mg, 0.46 mmol). The reaction mixture was heated at 80° C. for 12 h, and then allowed to cool to ambient temperature (about 20° C.) and partitioned between water and ethyl acetate (1:1, 200 mL). The organic layer was separated, dried over magnesium sulfate, filtered and concentrated under reduced pressure. The resulting material was purified by silica gel chromatography (20% ethyl acetate in hexanes as eluant) to provide the title compound (1.7 g).

¹H NMR (CDCl₃): δ 7.77 (m, 1H), 7.59 (m, 1H), 7.32 (m, 2H), 7.15 (m, 1H), 4.17 (m, 2H), 1.25 (m, 3H).

### Step C: Preparation of 4-chloro-1-(2-chloro-4-fluorophenyl)-1H-imidazole-5-carboxylic acid

To a mixture of ethyl 4-chloro-1-(2-chloro-4-fluorophenyl)-1H-imidazole-5-carboxylate (i.e. the product of Step B) (1.7 g, 5.6 mmol) in methanol (21 mL) and tetrahydrofuran (21 mL) was added dropwise sodium hydroxide (1 N, 27 mL). After 2 h, the reaction mixture was concentrated under reduced pressure and the resulting material was acidified to pH 2 with aqueous hydrochloric acid solution (6 N). The resulting mixture was extracted with ethyl acetate (3×100 mL) and the combined organic layers were washed with saturated aqueous sodium chloride solution, dried over sodium sulfate, filtered and concentrated under reduced pressure to provide the title compound as a white solid (1.48 g).

¹H NMR (DMSO-d₆): δ 7.75 (m, 1H), 7.73-7.68 (m, 2H), 7.32 (m, 1H).

### Step D: Preparation of 4-chloro-1-(2-chloro-4-fluorophenyl)-1H-imidazole-5-carbonyl chloride

To a mixture of 4-chloro-1-(2-chloro-4-fluorophenyl)-1H-imidazole-5-carboxylic acid (i.e. the product of Step C) (0.55 g, 2 mmol) in dichloromethane (5 mL) and N,N-dimethylformamide (catalytic amount) was added dropwise oxalyl chloride (0.5 mL, 6 mmol). The reaction mixture was heated at 40° C. for 2 h, and then concentrated under reduced pressure to provide the title compound (0.8 g), which was used without purification.

### Step E: Preparation of [4-chloro-1-(2-chloro-4-fluorophenyl)-1H-imidazol-5-yl](2,4-difluorophenyl)methanone

To a mixture of 4-chloro-1-(2-chloro-4-fluorophenyl)-1H-imidazole-5-carbonyl chloride (i.e. the product of Step D) (0.8 g, 2 mmol) in tetrachloroethane (10 mL) was added aluminum chloride (0.91 g, 6.8 mmol) and 1,3-difluorobenzene (1.3 mL, 13 mmol). The reaction mixture was heated at 150° C. for 48 h, cooled to ambient temperature (about 20° C.), poured into cold aqueous hydrochloric acid solution (1 N) and extracted with ethyl acetate. The organic layer was dried over sodium sulfate, filtered and concentrated under reduced pressure. The resulting material was purified by silica gel chromatography (10% ethyl acetate in hexanes as eluant) to provide the title compound as a yellow oil (0.3 g).

¹H NMR (CDCl₃): δ 7.60 (m, 1H), 7.52 (m, 1H), 7.42 (m, 1H), 7.32 (m, 1H), 7.18 (m, 1H), 6.98 (m, 1H), 6.92 (m, 1H).

### Step F: Preparation of 4-chloro-1-(2-chloro-4-fluorophenyl)-α-(2,4-difluorophenyl)-1H-imidazole-5-methanol

To a mixture of [4-chloro-1-(2-chloro-4-fluoroophenyl)-1H-imidazol-5-yl](2,4-difluorophenyl)methanone (i.e. the product of Step E) (0.24 g, 0.65 mmol) in methanol (10 mL) at 0° C. was added sodium borohydride (0.122 g, 3.23 mmol). The reaction mixture was allowed to warm to ambient temperature (about 20° C.) and stirred for 3 h. The reaction mixture was concentrated under reduced pressure and the resulting material was diluted with water and extracted with ethyl acetate (3×50 mL). The combined organic layers were dried over sodium sulfate, filtered and concentrated under reduced pressure. The resulting material was purified by silica gel chromatography (10% ethyl acetate in hexanes as eluant) to provide the title compound, a compound of the present invention, as a white solid (0.18 g).

¹H NMR (CDCl₃): δ 7.43-7.35 (m, 2H), 7.28 (m, 2H), 7.18 (m, 1H), 7.14-7.07 (m, 2H) 6.88 (m, 2H), 6.82 (m, 1H), 6.75 (m, 2H), 5.77 (m, 1H), 5.69 (m, 1H).

### Example 3

### Preparation of 2-chloro-1-(2-chloro-4,6-difluorophenyl)-α-(2,4-difluorophenyl)-α,4-dimethyl-1H-imidazole-5-methanol (Compound 81)

### Step A: Preparation of ethyl 1-(2-chloro-4,6-difluorophenyl)-4-methyl-1H-imidazole-5-carboxylate

To a mixture of 2-chloro-4,6-difluorobenzenamine (19.3 g, 118 mmol) in methanol (200 mL) was added ethyl glyoxylate (50% solution in toluene, 33.6 g, 164 mmol). The reaction mixture was heated at 65° C. for 1 h, and then concentrated under reduced pressure. The resulting material was concentrated onto silica gel and purified by column chromatography (1:1 dichloromethane and hexanes as eluant) to provide an oil (25.5 g). To a mixture of the oil in ethanol (200 mL) was added 1-[(isocyanoethyl)sulfonyl]-4-methylbenzene (19.6 g, 93.7 mmol) and powdered potassium carbonate (21 g, 152 mmol). The reaction mixture was heated at reflux for 2 h, and then concentrated under reduced pressure. The resulting material was partitioned between ethyl acetate and water, the layers were separated, and the aqueous layer was extracted with ethyl acetate (3×200 mL). The combined organic layers were dried over magnesium sulfate, filtered and concentrated under reduced pressure. The resulting material was purified by silica gel column chromatography (20% ethyl acetate in hexanes as eluant) to provide the title compound as an off-white solid (18 g).

¹H NMR (CDCl₃): δ 7.47 (s, 1H), 7.12 (m, 1H), 6.95 (m, 1H), 4.18 (q, 2H), 2.59 (s, 3H) 1.21 (t, 3H).

### Step B: Preparation of ethyl 2-chloro-1-(2-chloro-4,6-difluorophenyl)-4-methyl-1H-imidazole-5-carboxylate

To a mixture of ethyl 1-(2-chloro-4,6-difluorophenyl)-4-methyl-1H-imidazole-5-carboxylate (i.e. the product of Step A) (11.4 g, 37.9 mmol) in tetrahydrofuran (200 mL) at −40° C. was added 2,2,6,6-tetramethylpiperidinylmagnesium chloride lithium chloride complex (1.2 M in tetrahydrofuran, 40 mL) over 20 minutes. After the addition was complete, the reaction temperature was allowed to warm to −15° C. over 30 minutes, and maintained between −15 to −17° C. for 15 minutes, and then hexachloroethane (13.4 g, 56.6 mmol) was added to the reaction mixture. The reaction mixture was allowed to warm to ambient temperature (about 20° C.) over 30 minutes, and then diluted with saturated aqueous ammonium chloride solution. The organic layer was separated, and the aqueous layer was extracted with ethyl acetate (2×200 mL). The combined organic layers were dried over magnesium sulfate, filtered and concentrated under reduced pressure. The resulting material was purified by silica gel chromatography (20% ethyl acetate in hexanes as eluant) to provide the title compound (10.7 g) as a white solid.

¹H NMR (CDCl₃): δ 7.14 (m, 1H), 6.97 (m, 1H), 4.18 (q, 2H), 2.56 (s, 3H), 1.20 (t, 3H).

### Step C: Preparation of 2-chloro-1-(2-chloro-4,6-difluorophenyl)-4-methyl-1H-imidazole-5-carboxylic acid

To a mixture of ethyl 2-chloro-1-(2-chloro-4,6-difluorophenyl)-4-methyl-1H-imidazole-5-carboxylate (i.e. the product of Step B) (30.0 g, 89.6 mmol) in methanol (200 mL) and water (200 mL) was added aqueous sodium hydroxide (50%, 32 g). The reaction mixture was stirred at 40° C. for 12 h, and then diluted with water (200 mL) and concentrated under reduce pressure to about one-half the starting volume. The resulting mixture was diluted with water (300 mL), cooled in an ice bath, and the pH was adjusted to about 2 by the addition of concentrated hydrochloric acid. The resulting slurry was filtered, and the solid collected was washed with water and dried under vacuum to provide the title compound as a white solid (8.0 g).

¹H NMR (DMSO-d₆) δ 13.3 (br s, 1H), 7.73 (m, 2H), 2.45 (s, 3H).

### Step D: Preparation of 2-chloro-1-(2-chloro-4,6-difluorophenyl)-N-methoxy-N,4-dimethyl-1H-imidazole-5-carboxamide

To a mixture of 2-chloro-1-(2-chloro-4,6-difluorophenyl)-4-methyl-1H-imidazole-5-carboxylic acid (i.e. the product of Step C) (86.5 g, 281 mmol) in dichloromethane (800 mL) was added N,N-dimethylformamide (a few drops), followed by oxalyl chloride (38 g, 299 mmol) over 15 minutes. The reaction mixture was stirred for 40 minutes, and then N-methoxymethanamine hydrochloride (1:1) (also known as N,O-dimethylhydroxylamine hydrochloride) (31 g, 317 mmol) was added, followed by sodium carbonate (65 g, 613 mmol) portionwise. The reaction mixture was stirred for 12 h, diluted with water (500 mL), and the layers were separated. The aqueous layer was extracted with ethyl acetate (150 mL), and the combined organic layers were dried over magnesium sulfate, filtered and concentrated under reduced pressure to give an off-white solid. The solid was washed with hexanes (400 mL) and dried under vacuum to provide the title compound (92.6 g).

¹H NMR (CDCl₃): δ 7.12 (m, 1H), 6.96 (m, 1H), 3.62 (s, 3H), 3.25 (s, 3H), 2.36 (s, 3H).

### Step E: Preparation of [2-chloro-1-(2-chloro-4,6-difluorophenyl)-4-methyl-1H-imidazol-5-yl]-(2,4-difluorophenyl)methanone

To a mixture of 1-bromo-2,4-difluorobenzene (1.47 g, 7.7 mmol) in tetrahydrofuran (30 mL) at −40° C. was added isopropylmagnesium chloride (2.0 M in tetrahydrofuran, 3.3 mL) via syringe. The reaction mixture was warmed to −2.5° C. over 100 minutes, and then 2-chloro-1-(2-chloro-4,6-difluorophenyl)-N-methoxy-N,4-dimethyl-1H-imidazole-5-carboxamide (i.e. the product of Step D) (1.6 g, 4.8 mmol) was added. The reaction mixture was warmed to ambient temperature (about 20° C.) and stirred for 12 h. The reaction mixture was diluted with water (50 mL), the layers were separated, and the aqueous layer was extracted with ethyl acetate (50 mL). The combined organic layers were dried over magnesium sulfate, filtered and concentrated under reduced pressure to provide the title compound as an off-white solid (1.9 g).

¹H NMR (CDCl₃): δ 7.52 (q, 1H), 7.11 (dt, 1H), 6.98 (m, 2H), 6.90 (dt, 1H), 2.12 (s, 3H).

### Step F: Preparation of 2-chloro-1-(2-chloro-4,6-difluorophenyl)-α-(2,4-difluorophenyl)-α,4-dimethyl-1H-imidazole-5-methanol

A mixture of [2-chloro-1-(2-chloro-4,6-difluorophenyl)-4-methyl-1H-imidazol-5-yl]-(2,4-difluorophenyl)methanone (i.e. the product of Step E) (370 mg, 0.91 mmol) in tetrahydrofuran (10 mL) was cooled to −28° C., and then methyllithium complex (1.6 M in diethyl ether, 0.8 mL) was added via syringe. The reaction mixture was warmed to 0° C. over 30 minutes, and then diluted with saturated aqueous ammonium chloride solution (10 mL), and extracted with ethyl acetate (10 mL). The organic layer was dried over magnesium sulfate, filtered and concentrated under reduced pressure. The resulting material was purified by silica gel chromatography (30% ethyl acetate in hexanes as eluant) to provide the title, a compound of the present invention, as a solid (21 mg).

MS 419 (M+1).

### Example 4

### Preparation of α-(2-chloro-4-fluorophenyl)-2-methyl-1-(2,4,6-trifluorophenyl)-1H-imidazole-5-methanol (Compound 167)

### Step A: Preparation of N-(2,4,6-trifluorophenyl)ethanimidamide

To a mixture of 2,4,6-trifluorobenzenamine (3.68 g, 25.0 mmol) in acetonitrile (50 mL) was added, over ten minutes, hydrogen chloride gas (generated by the dropwise addition of concentrated sulfuric acid (10 mL) to stirred concentrated hydrochloric acid (10 mL) in a separate flask, with the gas thus generated being vented through a piece of Tygon® tubing fitted to a plastic pipet placed below the surface of the acetonitrile reaction mixture). The reaction mixture was stirred overnight (about 16 h) at ambient temperature (about 20° C.), and then concentrated under reduced pressure. The resulting white solid was suspended in dichloromethane (about 50 mL) and saturated aqueous sodium bicarbonate solution (about 50 mL) was slowly added, with agitation, until all solids were dissolved and gas evolution had ceased. The layers were separated, and the aqueous layer was extracted with dichloromethane (2×50 mL). The combined organic layers were dried over magnesium sulfate, filtered and concentrated under reduced pressure to provide the title compound as a white solid (4.0 g).

¹H NMR (CDCl₃): δ 6.7 (m, 2H), 5.0 and 4.6 (br s, 2H total), 2.19 and 1.83 (s, 3H total).

### Step B: Preparation of 2-methyl-1-(2,4,6-trifluorophenyl)-1H-imidazole-5-carboxaldehyde

To a mixture of N-(2,4,6-trifluorophenyl)ethanimidamide (i.e. the product of Step A) (4.00 g, 21.2 mmol) in isopropyl alcohol (40 mL) glacial acetic acid (1.44 mL, 25 mmol), triethylamine (3.35 mL, 24 mmol) and 2-bromopropanedial (3.22 g, 21.3 mmol) were added. The reaction mixture was heated at reflux for 1 h, and then water (about 40 mL) was added. The reaction mixture was concentrated under reduced pressure to about one-half the starting volume, and saturated aqueous sodium bicarbonate solution (50 mL) and ethyl acetate (100 mL) were added. The resulting mixture was filtered through a sintered glass frit funnel. The organic layer was separated, and the aqueous layer was extracted ethyl acetate (50 mL). The combined organic layers were washed with saturated sodium chloride solution, dried over magnesium sulfate, filtered and concentrated under reduced pressure to provide a brown solid. The solid was washed with a small amount of diethyl ether to provide the title compound as an off-white solid (3.4 g).

¹H NMR (CDCl₃): δ 9.66 (s, 1H), 7.84 (s, 1H), 6.88 (m, 2H), 2.31 (s, 3H).

### Step C: Preparation of α-(2-chloro-4-fluorophenyl)-2-methyl-1-(2,4,6-trifluorophenyl)-1H-imidazole-5-methanol

To a mixture of 1-bromo-2-chloro-4-fluorobenzene (2.35 mL, 19.3 mmol) in tetrahydrofuran (15 mL) at −2 to −3° C. was added isopropylmagnesium chloride lithium chloride (1.3 M in tetrahydrofuran, 15 mL, 19.5 mmol) dropwise over ten minutes. The reaction mixture was stirred for 1.5 h at 0 to 5° C., and then 2-methyl-1-(2,4,6-trifluorophenyl)-1H-imidazole-5-carboxaldehyde (i.e. the product of Step B) (2.32 g, 9.65 mmol) in tetrahydrofuran (8 mL) was added dropwise over 10 minutes while maintaining the reaction temperature at about 0 to 5° C. After 1 h, saturated aqueous ammonium chloride solution (10 mL) was added dropwise to the reaction mixture, and the mixture was extracted with ethyl acetate (2×25 mL). The combined organic layers were washed with aqueous sodium chloride solution, dried over magnesium sulfate, filtered and concentrated under reduced pressure at 45° C. until a slurry was obtained. Hexanes were added to the resulting slurry (with agitation) and the mixture was allowed to cool to ambient temperature (about 20° C.). The resulting precipitate was collected on a sintered glass frit funnel, washed ethyl acetate/hexanes (1:1, 3 mL), and allowed to air dry to provide the title compound, a compound of the present invention, as a tan solid (1.866 g).

¹H NMR (DMSO-d₆) δ δ 7.5 (m, 3H), 7.38 (m, 1H), 7.20 (m, 1H), 6.43 (s, 1H), 5.96 (m, 1H), 5.64 (m, 1H), 2.05 (s, 3H).

### Example 5

### Preparation of 4-bromo-α-(2-chloro-4-fluorophenyl)-2-methyl-1-(2,4,6-trifluorophenyl)-1H-imidazole-5-methanol (Compound 168)

To a mixture of α-(2-chloro-4-fluorophenyl)-2-methyl-1-(2,4,6-trifluorophenyl)-1H-imidazole-5-methanol (i.e. the product of Step C, Example 4) (1.796 g, 4.84 mmol) in N,N-dimethylformamide (15 mL) was added N-bromosuccinimide (0.905 g, 5.08 mmol) portionwise. The reaction mixture was stirred at ambient temperature (about 20° C.) for 5 h, and then water (1 mL), saturated aqueous sodium bisulfite solution (0.25 mL) and saturated aqueous sodium bicarbonate solution (0.25 mL) were added sequentially. Stirring was continued, and water (10 mL) was added dropwise until a suspension formed. After 10 minutes, more water (20 mL) was added. After 30 minutes, the precipitate that formed was collected on a sintered glass frit funnel and washed with water (5 mL) and aqueous methanol (33%, 5 mL). The solid was air dried to provide the title compound, a compound of the present invention, as a white solid (1.736 g).

¹H NMR (DMSO-d₆) δ 7.53 (m, 1H), 7.34 (m, 1H), 7.17 (m, 1H), 7.06 (m, 1H), 6.94 (m, 1H), 6.33 (m, 1H), 5.73 (m, 1H), 1.98 (s, 3H).

### Example 6

### Preparation of 4-bromo-1-(2,6-difluorophenyl)-α-(2-chloro-4-methoxyphenyl)-1H-imidazole-5-methanol (Compound 274)

### Step A: Preparation of 1-(2,6-difluorophenyl)-1H-imidazole-5-methanol

To a mixture of ethyl 1-(2,6-difluorophenyl)-1H-imidazole-5-carboxylate (7.00 g, 27.75 mmol, prepared by the method of Example 1, Step A) in tetrahydrofuran (100 mL) cooled in an ice-water bath was added lithium aluminum hydride (1.0 M in tetrahydrofuran, 27.8 mL, 27.8 mmol) dropwise. After 45 minutes, water (1.0 mL) was added to the reaction mixture, followed by sodium hydroxide (15% aqueous solution, 1.0 mL), and then more water (3.0 mL). The resulting mixture was stirred for 16 h, and then magnesium sulfate (small amount) was added, and the mixture was filtered through a pad of Celite® (diatomaceous filter aid) on a sintered glass frit funnel. The filtrate was concentrated under reduced pressure to provide the title compound as a white solid (5.57 g).

¹H NMR (CDCl₃): δ 7.52 (s, 1H), 7.45 (m, 1H), 7.11 (m, 2H), 4.53 (s, 2H), 2.15 (br s, 1H).

### Step B: Preparation of 1-(2,6-difluorophenyl)-1H-imidazole-5-carboxaldehyde

To a mixture of 1-(2,6-difluorophenyl)-1H-imidazole-5-methanol (i.e. the product of Step A) (3.4 g, 16.2 mmol) in dichloromethane (60 mL) was added manganese(IV) oxide (16.5 g, 162 mmol). The reaction mixture was heated at reflux for 3 h, cooled, and filtered through a pad of Celite® (diatomaceous filter aid) on a sintered glass frit funnel. The filtrate was concentrated under reduced pressure and the resulting solid was washed on a glass frit funnel with a small amount of diethyl ether and allowed to air dry to provide the title compound as a white solid (2.51 g).

¹H NMR (CDCl₃): δ 9.79 (d, J=0.8 Hz, 1H), 7.96 (d, J=0.8 Hz, 1H), 7.75 (s, 1H), 7.45 (m, 1H), 7.12 (m, 2H).

### Step C: Preparation of α-(2-chloro-4-methoxyphenyl)-1-(2,6-difluorophenyl)-1H-imidazole-5-methanol

To a mixture of 1-bromo-2-chloro-4-methoxybenzene (5.31 g, 24.0 mmol) in tetrahydrofuran (40 mL) cooled in an ice-water bath was added dropwise isopropylmagnesium chloride lithium chloride complex (1.3 M tetrahydrofuran, 18.4 mL, 24.0 mmol) over 15 minutes. The reaction mixture was allowed to warm to ambient temperature (about 20° C.) and stirred for 16 h. After 16 h, the reaction mixture was cooled to 0° C. and 1-(2,6-difluorophenyl)-1H-imidazole-5-carboxaldehyde (i.e. the product of Step B) (2.50 g, 12.0 mmol) in tetrahydrofuran (10 mL) was added dropwise. The reaction mixture was stirred for about 15 minutes, and then saturated aqueous ammonium chloride solution (about 3 mL) was added. After about 5 minutes more saturated aqueous ammonium chloride solution (about 100 mL) was added and the resulting mixture was extracted with ethyl acetate (100 mL). The organic layer was washed with saturated aqueous sodium chloride solution, dried over magnesium sulfate, filtered and concentrated under reduced pressure to provide the title compound as a white solid (2.77 g).

¹H NMR (CDCl₃): δ 7.51 (s, 1H), 7.43 (m, 1H), 7.38 (d, 1H) 7.1-7.0 (m, 2H), 6.9 (m, 1H), 6.82 (m, 1H), 6.78 (m, 1H), 5.98 (m, 1H), 3.80 (s, 3H), 2.4 (m, 1H).

### Step D: Preparation of 4-bromo-α-(2-chloro-4-methoxyphenyl)-1-(2,6-difluorophenyl)-1H-imidazole-5-methanol

To α-(2-chloro-4-methoxyphenyl)-1-(2,6-difluorophenyl)-1H-imidazole-5-methanol (i.e. the product of Step C) (1.72 g, 4.90 mmol) in N,N-dimethylformamide (15 mL) was added N-bromosuccinimide (0.91 g, 5.11 mmol). The reaction mixture was stirred for 16 h, and then heated at 40° C. for 16 h. Additional N-bromosuccinimide (0.31 g, 1.74 mmol) was added to the reaction mixture and the mixture was heated at 40° C. for 2 h and at 60° C. for 10 h. The reaction mixture was diluted with water, stirred for 30 minutes and filtered. The solid collected was washed with water, a small amount of water/methanol (1:1 mixture) and allowed to air dry to provide the title compound, a compound of the present invention, as a white solid (1.43 g).

¹H NMR (CDCl₃): δ 7.37 (m, 1H), 7.34 (s, 1H), 7.1-7.0 (m, 2H), 6.81 (m, 1H), 6.76 (m, 1H), 6.48 (m, 1H), 6.07 (m, 1H), 3.75 (s, 3H), 2.38 (m, 1H).

### Example 7

### Preparation of 4-bromo-2-chloro-α-(2-chloro-4-methoxyphenyl)-1-(2,6-difluorophenyl)-1H-imidazole-5-methanol (Compound 287)

To 4-bromo-α-(2-chloro-4-methoxyphenyl)-1-(2,6-difluorophenyl)-1H-imidazole-5-methanol (i.e. the product of Example 6) (1.34 g, 3.13 mmol) in N,N-dimethylformamide (6 mL) was added N-chlorosuccinimide (0.44 g, 3.30 mmol). The reaction mixture was heated at 40° C. for 16 h. Additional N-chlorosuccinimide (0.083 g, 0.62 mmol) was added to the reaction mixture and the mixture was heated at 40° C. for 24 h. The reaction mixture was diluted with water, stirred for 30 minutes and filtered. The solid collected was washed water, a small amount of water/methanol (1:1 mixture) and allowed to air dry to provide the title compound, a compound of the present invention, as a white solid (0.45 g).

¹H NMR (CDCl₃): δ 7.40 (m, 1H), 7.06 (m, 1H), 6.98 (m, 1H), 6.82 (m, 1H), 6.75 (m, 1H), 6.42 (m, 1H), 6.00 (m, 1H), 3.76 (s, 3H), 2.39 (m, 1H).

By the procedures described herein together with methods known in the art, the compounds disclosed in the Tables that follow can be prepared. The following abbreviations are used in the Tables which follow: Me means methyl, Et means ethyl, MeO means methoxy, EtO means ethoxy, Ph means phenyl and CN means cyano.

TABLE 1 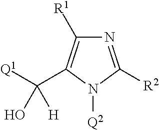
Q¹ is 4-F—Ph, R¹ is H, R² is Me.

| Q² | Q² | Q² | Q² | Q² |
|---|---|---|---|---|
| 2-Br—Ph | 2-I-4-F—Ph | 2-F-4-MeO—Ph | 2,4-di-Cl-6-F—Ph | 2-Br-4-Cl-6-F—Ph |
| 2-Cl—Ph | 2-I-6-F—Ph | 2-F-4-EtO—Ph | 2,6-di-Cl-4-CN—Ph | 2-Br-4-F-6-Cl—Ph |
| 2-F—Ph | 2-Cl-4-Br—Ph | 2-Br-4-EtO—Ph | 2,6-di-Cl-4-F—Ph | 2-Cl-4-Br-6-F—Ph |
| 2-I—Ph | 2-Cl-4-CN—Ph | 2-Cl-4-EtO—Ph | 2,6-di-Cl-4-MeO—Ph | 2-Br-3-pyridinyl |
| 2-CF₃—Ph | 2-Cl-4-F—Ph | 2-CF₃-4-F—Ph | 2,6-di-F-4-Br—Ph | 2-Cl-3-pyridinyl |
| 2,4-di-Cl—Ph | 2-Cl-6-F—Ph | 2-CF₃-6-F—Ph | 2,6-di-F-4-Cl—Ph | 2-Me-3-pyridinyl |
| 2,6-di-Cl—Ph | 2-Cl-4-I—Ph | 2,4,6-tri-Cl—Ph | 2,6-di-F-4-CN—Ph | 2,4-di-Cl-3-pyridinyl |
| 2,4-di-F—Ph | 2,3,5-tri-F—Ph | 2-Cl-4-MeO—Ph | 2,6-di-F-4-I—Ph | 2,6-di-Cl-3-pyridinyl |
| 2,6-di-F—Ph | 2-F-4-Br—Ph | 2,3,6-tri-F—Ph | 2,6-di-F-4-MeO—Ph | 3,5-di-Cl-2-pyridinyl |
| 2-Br-4-Cl—Ph | 2-F-4-Cl—Ph | 2,4,5-tri-F—Ph | 2,6-di-F-4-EtO—Ph | 3,5-di-F-2-pyridinyl |
| 2-Br-4-CN—Ph | 2-F-4-CN—Ph | 2,4,6-tri-F—Ph | 2-Br-4,6-di-F—Ph | 2-Cl-6-MeO-3-pyridinyl |
| 2-Br-4-F—Ph | 2-F-4-I—Ph | 2,4-di-Br-6-F—Ph | 2-Cl-4,6-di-F—Ph | 1,3-di-Me-1H-4-pyrazol-5-yl |
| 2-Br-6-F—Ph | 2-Me-4-F—Ph | 2-Br-4-MeO—Ph | 2-I-4,6-di-F—Ph | 2-Br-3-thienyl |

The present disclosure also includes Tables 1A through 356A, each of which is constructed the same as Table 1 above, except that the row heading in Table 1 (i.e. “Q¹ is 4-F-Ph, R¹ is H, R² is Me”) is replaced with the respective row heading shown below. For Example, in Table 1A the row heading is “Q¹ is 4-F-Ph, R¹ is H, R² is Br”, and Q² is as defined in Table 1 above. Thus, the first entry in Table 1A specifically discloses 2-bromo-α-(4-fluorophenyl)-1-(2-bromophenyl)-1H-imidazole-5-methanol. Tables 2A through 356A are constructed similarly.

| Table | Row Heading |
|---|---|
| 1A | Q¹ is 4-F—Ph, R¹ is H, R² is Br. |
| 2A | Q¹ is 4-F—Ph, R¹ is H, R² is Cl. |
| 3A | Q¹ is 4-F—Ph, R¹ is Br, R² is H. |
| 4A | Q¹ is 4-F—Ph, R¹ is Br, R² is Br. |
| 5A | Q¹ is 4-F—Ph, R¹ is Br, R² is Cl. |
| 6A | Q¹ is 4-F—Ph, R¹ is Br, R² is Me. |
| 7A | Q¹ is 4-F—Ph, R¹ is Cl, R² is H. |
| 8A | Q¹ is 4-F—Ph, R¹ is Cl, R² is Br. |
| 9A | Q¹ is 4-F—Ph, R¹ is Cl, R² is Cl. |
| 10A | Q¹ is 4-F—Ph, R¹ is Cl, R² is Me. |
| 11A | Q¹ is 4-F—Ph, R¹ is Me, R² is H. |
| 12A | Q¹ is 4-F—Ph, R¹ is Me, R² is Br. |
| 13A | Q¹ is 4-F—Ph, R¹ is Me, R² is Cl. |
| 14A | Q¹ is 4-F—Ph, R¹ is Me, R² is Me. |
| 15A | Q¹ is 2,4-di-F—Ph, R¹ is H, R² is Br. |
| 16A | Q¹ is 2,4-di-F—Ph, R¹ is H, R² is Cl. |
| 17A | Q¹ is 2,4-di-F—Ph, R¹ is H, R² is Me. |
| 18A | Q¹ is 2,4-di-F—Ph, R¹ is Br, R² is H. |
| 19A | Q¹ is 2,4-di-F—Ph, R¹ is Br, R² is Br. |
| 20A | Q¹ is 2,4-di-F—Ph, R¹ is Br, R² is Cl. |
| 21A | Q¹ is 2,4-di-F—Ph, R¹ is Br, R² is Me. |
| 22A | Q¹ is 2,4-di-F—Ph, R¹ is Cl, R² is H. |
| 23A | Q¹ is 2,4-di-F—Ph, R¹ is Cl, R² is Br. |
| 24A | Q¹ is 2,4-di-F—Ph, R¹ is Cl, R² is Cl. |
| 25A | Q¹ is 2,4-di-F—Ph, R¹ is Cl, R² is Me. |
| 26A | Q¹ is 2,4-di-F—Ph, R¹ is Me, R² is H. |
| 27A | Q¹ is 2,4-di-F—Ph, R¹ is Me, R² is Br. |
| 28A | Q¹ is 2,4-di-F—Ph, R¹ is Me, R² is Cl. |
| 29A | Q¹ is 2,4-di-F—Ph, R¹ is Me, R² is Me. |
| 30A | Q¹ is 2-Cl-4-F—Ph, R¹ is H, R² is Br. |
| 31A | Q¹ is 2-Cl-4-F—Ph, R¹ is H, R² is Cl. |
| 32A | Q¹ is 2-Cl-4-F—Ph, R¹ is H, R² is Me. |
| 33A | Q¹ is 2-Cl-4-F—Ph, R¹ is Br, R² is H. |
| 34A | Q¹ is 2-Cl-4-F—Ph, R¹ is Br, R² is Br. |
| 35A | Q¹ is 2-Cl-4-F—Ph, R¹ is Br, R² is Cl. |
| 36A | Q¹ is 2-Cl-4-F—Ph, R¹ is Br, R² is Me. |
| 37A | Q¹ is 2-Cl-4-F—Ph, R¹ is Cl, R² is H. |
| 38A | Q¹ is 2-Cl-4-F—Ph, R¹ is Cl, R² is Br. |
| 39A | Q¹ is 2-Cl-4-F—Ph, R¹ is Cl, R² is Cl. |
| 40A | Q¹ is 2-Cl-4-F—Ph, R¹ is Cl, R² is Me. |
| 41A | Q¹ is 2-Cl-4-F—Ph, R¹ is Me, R² is H. |
| 42A | Q¹ is 2-Cl-4-F—Ph, R¹ is Me, R² is Br. |
| 43A | Q¹ is 2-Cl-4-F—Ph, R¹ is Me, R² is Cl. |
| 44A | Q¹ is 2-Cl-4-F—Ph, R¹ is Me, R² is Me. |
| 45A | Q¹ is 2,4-di-Cl—Ph, R¹ is H, R² is Br. |
| 46A | Q¹ is 2,4-di-Cl—Ph, R¹ is H, R² is Cl. |
| 47A | Q¹ is 2,4-di-Cl—Ph, R¹ is H, R² is Me. |
| 48A | Q¹ is 2,4-di-Cl—Ph, R¹ is Br, R² is H. |
| 49A | Q¹ is 2,4-di-Cl—Ph, R¹ is Br, R² is Br. |
| 50A | Q¹ is 2,4-di-Cl—Ph, R¹ is Br, R² is Cl. |
| 51A | Q¹ is 2,4-di-Cl—Ph, R¹ is Br, R² is Me. |
| 52A | Q¹ is 2,4-di-Cl—Ph, R¹ is Cl, R² is H. |
| 53A | Q¹ is 2,4-di-Cl—Ph, R¹ is Cl, R² is Br. |
| 54A | Q¹ is 2,4-di-Cl—Ph, R¹ is Cl, R² is Cl. |
| 55A | Q¹ is 2,4-di-Cl—Ph, R¹ is Cl, R² is Me. |
| 56A | Q¹ is 2,4-di-Cl—Ph, R¹ is Me, R² is H. |
| 57A | Q¹ is 2,4-di-Cl—Ph, R¹ is Me, R² is Br. |
| 58A | Q¹ is 2,4-di-Cl—Ph, R¹ is Me, R² is Cl. |
| 59A | Q¹ is 2,4-di-Cl—Ph, R¹ is Me, R² is Me. |
| 60A | Q¹ is 2-F-4-Cl—Ph, R¹ is H, R² is Br. |
| 61A | Q¹ is 2-F-4-Cl—Ph, R¹ is H, R² is Cl. |
| 62A | Q¹ is 2-F-4-Cl—Ph, R¹ is H, R² is Me. |
| 63A | Q¹ is 2-F-4-Cl—Ph, R¹ is Br, R² is H. |
| 64A | Q¹ is 2-F-4-Cl—Ph, R¹ is Br, R² is Br. |
| 65A | Q¹ is 2-F-4-Cl—Ph, R¹ is Br, R² is Cl. |
| 66A | Q¹ is 2-F-4-Cl—Ph, R¹ is Br, R² is Me. |
| 67A | Q¹ is 2-F-4-Cl—Ph, R¹ is Cl, R² is H. |
| 68A | Q¹ is 2-F-4-Cl—Ph, R¹ is Cl, R² is Br. |
| 69A | Q¹ is 2-F-4-Cl—Ph, R¹ is Cl, R² is Cl. |
| 70A | Q¹ is 2-F-4-Cl—Ph, R¹ is Cl, R² is Me. |
| 71A | Q¹ is 2-F-4-Cl—Ph, R¹ is Me, R² is H. |
| 72A | Q¹ is 2-F-4-Cl—Ph, R¹ is Me, R² is Br. |
| 73A | Q¹ is 2-F-4-Cl—Ph, R¹ is Me, R² is Cl. |
| 74A | Q¹ is 2-F-4-Cl—Ph, R¹ is Me, R² is Me. |
| 75A | Q¹ is 2-Br-4-F—Ph, R¹ is H, R² is Br. |
| 76A | Q¹ is 2-Br-4-F—Ph, R¹ is H, R² is Cl. |
| 77A | Q¹ is 2-Br-4-F—Ph, R¹ is H, R² is Me. |
| 78A | Q¹ is 2-Br-4-F—Ph, R¹ is Br, R² is H. |
| 79A | Q¹ is 2-Br-4-F—Ph, R¹ is Br, R² is Br. |
| 80A | Q¹ is 2-Br-4-F—Ph, R¹ is Br, R² is Cl. |
| 81A | Q¹ is 2-Br-4-F—Ph, R¹ is Br, R² is Me. |
| 82A | Q¹ is 2-Br-4-F—Ph, R¹ is Cl, R² is H. |
| 83A | Q¹ is 2-Br-4-F—Ph, R¹ is Cl, R² is Br. |
| 84A | Q¹ is 2-Br-4-F—Ph, R¹ is Cl, R² is Cl. |
| 85A | Q¹ is 2-Br-4-F—Ph, R¹ is Cl, R² is Me. |
| 86A | Q¹ is 2-Br-4-F—Ph, R¹ is Me, R² is H. |
| 87A | Q¹ is 2-Br-4-F—Ph, R¹ is Me, R² is Br. |
| 88A | Q¹ is 2-Br-4-F—Ph, R¹ is Me, R² is Cl. |
| 89A | Q¹ is 2-Br-4-F—Ph, R¹ is Me, R² is Me. |
| 90A | Q¹ is 2-Me-4-F—Ph, R¹ is H, R² is Br. |
| 91A | Q¹ is 2-Me-4-F—Ph, R¹ is H, R² is Cl. |
| 92A | Q¹ is 2-Me-4-F—Ph, R¹ is H, R² is Me. |
| 93A | Q¹ is 2-Me-4-F—Ph, R¹ is Br, R² is H. |
| 94A | Q¹ is 2-Me-4-F—Ph, R¹ is Br, R² is Br. |
| 95A | Q¹ is 2-Me-4-F—Ph, R¹ is Br, R² is Cl. |
| 96A | Q¹ is 2-Me-4-F—Ph, R¹ is Br, R² is Me. |
| 97A | Q¹ is 2-Me-4-F—Ph, R¹ is Cl, R² is H. |
| 98A | Q¹ is 2-Me-4-F—Ph, R¹ is Cl, R² is Br. |
| 99A | Q¹ is 2-Me-4-F—Ph, R¹ is Cl, R² is Cl. |
| 100A | Q¹ is 2-Me-4-F—Ph, R¹ is Cl, R² is Me. |
| 101A | Q¹ is 2-Me-4-F—Ph, R¹ is Me, R² is H. |
| 102A | Q¹ is 2-Me-4-F—Ph, R¹ is Me, R² is Br. |
| 103A | Q¹ is 2-Me-4-F—Ph, R¹ is Me, R² is Cl. |
| 104A | Q¹ is 2-Me-4-F—Ph, R¹ is Me, R² is Me. |
| 105A | Q¹ is 2,4,6-tri-F—Ph, R¹ is H, R² is Br. |
| 106A | Q¹ is 2,4,6-tri-F—Ph, R¹ is H, R² is Cl. |
| 107A | Q¹ is 2,4,6-tri-F—Ph, R¹ is H, R² is Me. |
| 108A | Q¹ is 2,4,6-tri-F—Ph, R¹ is Br, R² is H. |
| 109A | Q¹ is 2,4,6-tri-F—Ph, R¹ is Br, R² is Br. |
| 110A | Q¹ is 2,4,6-tri-F—Ph, R¹ is Br, R² is Cl. |
| 111A | Q¹ is 2,4,6-tri-F—Ph, R¹ is Br, R² is Me. |
| 112A | Q¹ is 2,4,6-tri-F—Ph, R¹ is Cl, R² is H. |
| 113A | Q¹ is 2,4,6-tri-F—Ph, R¹ is Cl, R² is Br. |
| 114A | Q¹ is 2,4,6-tri-F—Ph, R¹ is Cl, R² is Cl. |
| 115A | Q¹ is 2,4,6-tri-F—Ph, R¹ is Cl, R² is Me. |
| 116A | Q¹ is 2,4,6-tri-F—Ph, R¹ is Me, R² is H. |
| 117A | Q¹ is 2,4,6-tri-F—Ph, R¹ is Me, R² is Br. |
| 118A | Q¹ is 2,4,6-tri-F—Ph, R¹ is Me, R² is Cl. |
| 119A | Q¹ is 2,4,6-tri-F—Ph, R¹ is Me, R² is Me. |
| 120A | Q¹ is 4-Cl—Ph, R¹ is H, R² is Br. |
| 121A | Q¹ is 4-Cl—Ph, R¹ is H, R² is Cl. |
| 122A | Q¹ is 4-Cl—Ph, R¹ is H, R² is Me. |
| 123A | Q¹ is 4-Cl—Ph, R¹ is Br, R² is H. |
| 124A | Q¹ is 4-Cl—Ph, R¹ is Br, R² is Br. |
| 125A | Q¹ is 4-Cl—Ph, R¹ is Br, R² is Cl. |
| 126A | Q¹ is 4-Cl—Ph, R¹ is Br, R² is Me. |
| 127A | Q¹ is 4-Cl—Ph, R¹ is Cl, R² is H. |
| 128A | Q¹ is 4-Cl—Ph, R¹ is Cl, R² is Br. |
| 129A | Q¹ is 4-Cl—Ph, R¹ is Cl, R² is Cl. |
| 130A | Q¹ is 4-Cl—Ph, R¹ is Cl, R² is Me. |
| 131A | Q¹ is 4-Cl—Ph, R¹ is Me, R² is H. |
| 132A | Q¹ is 4-Cl—Ph, R¹ is Me, R² is Br. |
| 133A | Q¹ is 4-Cl—Ph, R¹ is Me, R² is Cl. |
| 134A | Q¹ is 4-Cl—Ph, R¹ is Me, R² is Me. |
| 135A | Q¹ is 2,6-di-F—Ph, R¹ is H, R² is Br. |
| 136A | Q¹ is 2,6-di-F—Ph, R¹ is H, R² is Cl. |
| 137A | Q¹ is 2,6-di-F—Ph, R¹ is H, R² is Me. |
| 138A | Q¹ is 2,6-di-F—Ph, R¹ is Br, R² is H. |
| 139A | Q¹ is 2,6-di-F—Ph, R¹ is Br, R² is Br. |
| 140A | Q¹ is 2,6-di-F—Ph, R¹ is Br, R² is Cl. |
| 141A | Q¹ is 2,6-di-F—Ph, R¹ is Br, R² is Me. |
| 142A | Q¹ is 2,6-di-F—Ph, R¹ is Cl, R² is H. |
| 143A | Q¹ is 2,6-di-F—Ph, R¹ is Cl, R² is Br. |
| 144A | Q¹ is 2,6-di-F—Ph, R¹ is Cl, R² is Cl. |
| 145A | Q¹ is 2,6-di-F—Ph, R¹ is Cl, R² is Me. |
| 146A | Q¹ is 2,6-di-F—Ph, R¹ is Me, R² is H. |
| 147A | Q¹ is 2,6-di-F—Ph, R¹ is Me, R² is Br. |
| 148A | Q¹ is 2,6-di-F—Ph, R¹ is Me, R² is Cl. |
| 149A | Q¹ is 2,6-di-F—Ph, R¹ is Me, R² is Me. |
| 150A | Q¹ is 2,4,6-tri-Cl—Ph, R¹ is H, R² is Br. |
| 151A | Q¹ is 2,4,6-tri-Cl—Ph, R¹ is H, R² is Cl. |
| 152A | Q¹ is 2,4,6-tri-Cl—Ph, R¹ is H, R² is Me. |
| 153A | Q¹ is 2,4,6-tri-Cl—Ph, R¹ is Br, R² is H. |
| 154A | Q¹ is 2,4,6-tri-Cl—Ph, R¹ is Br, R² is Br. |
| 155A | Q¹ is 2,4,6-tri-Cl—Ph, R¹ is Br, R² is Cl. |
| 156A | Q¹ is 2,4,6-tri-Cl—Ph, R¹ is Br, R² is Me. |
| 157A | Q¹ is 2,4,6-tri-Cl—Ph, R¹ is Cl, R² is H. |
| 158A | Q¹ is 2,4,6-tri-Cl—Ph, R¹ is Cl, R² is Br. |
| 159A | Q¹ is 2,4,6-tri-Cl—Ph, R¹ is Cl, R² is Cl. |
| 160A | Q¹ is 2,4,6-tri-Cl—Ph, R¹ is Cl, R² is Me. |
| 161A | Q¹ is 2,4,6-tri-Cl—Ph, R¹ is Me, R² is H. |
| 162A | Q¹ is 2,4,6-tri-Cl—Ph, R¹ is Me, R² is Br. |
| 163A | Q¹ is 2,4,6-tri-Cl—Ph, R¹ is Me, R² is Cl. |
| 164A | Q¹ is 2,4,6-tri-Cl—Ph, R¹ is Me, R² is Me. |
| 165A | Q¹ is 2-Cl-4,6-di-F—Ph, R¹ is H, R² is Br. |
| 166A | Q¹ is 2-Cl-4,6-di-F—Ph, R¹ is H, R² is Cl. |
| 167A | Q¹ is 2-Cl-4,6-di-F—Ph, R¹ is H, R² is Me. |
| 168A | Q¹ is 2-Cl-4,6-di-F—Ph, R¹ is Br, R² is H. |
| 169A | Q¹ is 2-Cl-4,6-di-F—Ph, R¹ is Br, R² is Br. |
| 170A | Q¹ is 2-Cl-4,6-di-F—Ph, R¹ is Br, R² is Cl. |
| 171A | Q¹ is 2-Cl-4,6-di-F—Ph, R¹ is Br, R² is Me. |
| 172A | Q¹ is 2-Cl-4,6-di-F—Ph, R¹ is Cl, R² is H. |
| 173A | Q¹ is 2-Cl-4,6-di-F—Ph, R¹ is Cl, R² is Br. |
| 174A | Q¹ is 2-Cl-4,6-di-F—Ph, R¹ is Cl, R² is Cl. |
| 175A | Q¹ is 2-Cl-4,6-di-F—Ph, R¹ is Cl, R² is Me. |
| 176A | Q¹ is 2-Cl-4,6-di-F—Ph, R¹ is Me, R² is H. |
| 177A | Q¹ is 2-Cl-4,6-di-F—Ph, R¹ is Me, R² is Br. |
| 178A | Q¹ is 2-Cl-4,6-di-F—Ph, R¹ is Me, R² is Cl. |
| 179A | Q¹ is 2-Cl-4,6-di-F—Ph, R¹ is Me, R² is Me. |
| 180A | Q¹ is 2,4-di-Cl-6-F—Ph, R¹ is H, R² is Br. |
| 181A | Q¹ is 2,4-di-Cl-6-F—Ph, R¹ is H, R² is Cl. |
| 182A | Q¹ is 2,4-di-Cl-6-F—Ph, R¹ is H, R² is Me. |
| 183A | Q¹ is 2,4-di-Cl-6-F—Ph, R¹ is Br, R² is H. |
| 184A | Q¹ is 2,4-di-Cl-6-F—Ph, R¹ is Br, R² is Br. |
| 185A | Q¹ is 2,4-di-Cl-6-F—Ph, R¹ is Br, R² is Cl. |
| 186A | Q¹ is 2,4-di-Cl-6-F—Ph, R¹ is Br, R² is Me. |
| 187A | Q¹ is 2,4-di-Cl-6-F—Ph, R¹ is Cl, R² is H. |
| 188A | Q¹ is 2,4-di-Cl-6-F—Ph, R¹ is Cl, R² is Br. |
| 189A | Q¹ is 2,4-di-Cl-6-F—Ph, R¹ is Cl, R² is Cl. |
| 190A | Q¹ is 2,4-di-Cl-6-F—Ph, R¹ is Cl, R² is Me. |
| 191A | Q¹ is 2,4-di-Cl-6-F—Ph, R¹ is Me, R² is H. |
| 192A | Q¹ is 2,4-di-Cl-6-F—Ph, R¹ is Me, R² is Br. |
| 193A | Q¹ is 2,4-di-Cl-6-F—Ph, R¹ is Me, R² is Cl. |
| 194A | Q¹ is 2,4-di-Cl-6-F—Ph, R¹ is Me, R² is Me. |
| 195A | Q¹ is 2-Br-4,6-di-F—Ph, R¹ is H, R² is Br. |
| 196A | Q¹ is 2-Br-4,6-di-F—Ph, R¹ is H, R² is Cl. |
| 197A | Q¹ is 2-Br-4,6-di-F—Ph, R¹ is H, R² is Me. |
| 198A | Q¹ is 2-Br-4,6-di-F—Ph, R¹ is Br, R² is H. |
| 199A | Q¹ is 2-Br-4,6-di-F—Ph, R¹ is Br, R² is Br. |
| 200A | Q¹ is 2-Br-4,6-di-F—Ph, R¹ is Br, R² is Cl. |
| 201A | Q¹ is 2-Br-4,6-di-F—Ph, R¹ is Br, R² is Me. |
| 202A | Q¹ is 2-Br-4,6-di-F—Ph, R¹ is Cl, R² is H. |
| 203A | Q¹ is 2-Br-4,6-di-F—Ph, R¹ is Cl, R² is Br. |
| 204A | Q¹ is 2-Br-4,6-di-F—Ph, R¹ is Cl, R² is Cl. |
| 205A | Q¹ is 2-Br-4,6-di-F—Ph, R¹ is Cl, R² is Me. |
| 206A | Q¹ is 2-Br-4,6-di-F—Ph, R¹ is Me, R² is H. |
| 207A | Q¹ is 2-Br-4,6-di-F—Ph, R¹ is Me, R² is Br. |
| 208A | Q¹ is 2-Br-4,6-di-F—Ph, R¹ is Me, R² is Cl. |
| 209A | Q¹ is 2-Br-4,6-di-F—Ph, R¹ is Me, R² is Me. |
| 210A | Q¹ is 2-F-4-MeO—Ph, R¹ is H, R² is Br. |
| 211A | Q¹ is 2-F-4-MeO—Ph, R¹ is H, R² is Cl. |
| 212A | Q¹ is 2-F-4-MeO—Ph, R¹ is H, R² is Me. |
| 213A | Q¹ is 2-F-4-MeO—Ph, R¹ is Br, R² is H. |
| 214A | Q¹ is 2-F-4-MeO—Ph, R¹ is Br, R² is Br. |
| 215A | Q¹ is 2-F-4-MeO—Ph, R¹ is Br, R² is Cl. |
| 216A | Q¹ is 2-F-4-MeO—Ph, R¹ is Br, R² is Me. |
| 217A | Q¹ is 2-F-4-MeO—Ph, R¹ is Cl, R² is H. |
| 218A | Q¹ is 2-F-4-MeO—Ph, R¹ is Cl, R² is Br. |
| 219A | Q¹ is 2-F-4-MeO—Ph, R¹ is Cl, R² is Cl. |
| 220A | Q¹ is 2-F-4-MeO—Ph, R¹ is Cl, R² is Me. |
| 221A | Q¹ is 2-F-4-MeO—Ph, R¹ is Me, R² is H. |
| 222A | Q¹ is 2-F-4-MeO—Ph, R¹ is Me, R² is Br. |
| 223A | Q¹ is 2-F-4-MeO—Ph, R¹ is Me, R² is Cl. |
| 224A | Q¹ is 2-F-4-MeO—Ph, R¹ is Me, R² is Me. |
| 225A | Q¹ is 2-Cl-4-MeO—Ph, R¹ is H, R² is Br. |
| 226A | Q¹ is 2-Cl-4-MeO—Ph, R¹ is H, R² is Cl. |
| 227A | Q¹ is 2-Cl-4-MeO—Ph, R¹ is H, R² is Me. |
| 228A | Q¹ is 2-Cl-4-MeO—Ph, R¹ is Br, R² is H. |
| 229A | Q¹ is 2-Cl-4-MeO—Ph, R¹ is Br, R² is Br. |
| 230A | Q¹ is 2-Cl-4-MeO—Ph, R¹ is Br, R² is Cl. |
| 231A | Q¹ is 2-Cl-4-MeO—Ph, R¹ is Br, R² is Me. |
| 232A | Q¹ is 2-Cl-4-MeO—Ph, R¹ is Cl, R² is H. |
| 233A | Q¹ is 2-Cl-4-MeO—Ph, R¹ is Cl, R² is Br. |
| 234A | Q¹ is 2-Cl-4-MeO—Ph, R¹ is Cl, R² is Cl. |
| 235A | Q¹ is 2-Cl-4-MeO—Ph, R¹ is Cl, R² is Me. |
| 236A | Q¹ is 2-Cl-4-MeO—Ph, R¹ is Me, R² is H. |
| 237A | Q¹ is 2-Cl-4-MeO—Ph, R¹ is Me, R² is Cl. |
| 238A | Q¹ is 2-Cl-4-MeO—Ph, R¹ is Me, R² is Me. |
| 239A | Q¹ is 2-F-4-CN—Ph, R¹ is H, R² is Br. |
| 240A | Q¹ is 2-F-4-CN—Ph, R¹ is H, R² is Cl. |
| 241A | Q¹ is 2-F-4-CN—Ph, R¹ is H, R² is Me. |
| 242A | Q¹ is 2-F-4-CN—Ph, R¹ is Br, R² is H. |
| 243A | Q¹ is 2-F-4-CN—Ph, R¹ is Br, R² is Br. |
| 244A | Q¹ is 2-F-4-CN—Ph, R¹ is Br, R² is Cl. |
| 245A | Q¹ is 2-F-4-CN—Ph, R¹ is Br, R² is Me. |
| 246A | Q¹ is 2-F-4-CN—Ph, R¹ is Cl, R² is H. |
| 247A | Q¹ is 2-F-4-CN—Ph, R¹ is Cl, R² is Br. |
| 248A | Q¹ is 2-F-4-CN—Ph, R¹ is Cl, R² is Cl. |
| 249A | Q¹ is 2-F-4-CN—Ph, R¹ is Cl, R² is Me. |
| 250A | Q¹ is 2-F-4-CN—Ph, R¹ is Me, R² is H. |
| 251A | Q¹ is 2-F-4-CN—Ph, R¹ is Me, R² is Br. |
| 252A | Q¹ is 2-F-4-CN—Ph, R¹ is Me, R² is Cl. |
| 253A | Q¹ is 2-F-4-CN—Ph, R¹ is Me, R² is Me. |
| 254A | Q¹ is 2-Cl-4-CN—Ph, R¹ is H, R² is Br. |
| 255A | Q¹ is 2-Cl-4-CN—Ph, R¹ is H, R² is Cl. |
| 256A | Q¹ is 2-Cl-4-CN—Ph, R¹ is H, R² is Me. |
| 257A | Q¹ is 2-Cl-4-CN—Ph, R¹ is Br, R² is H. |
| 258A | Q¹ is 2-Cl-4-CN—Ph, R¹ is Br, R² is Br. |
| 259A | Q¹ is 2-Cl-4-CN—Ph, R¹ is Br, R² is Cl. |
| 260A | Q¹ is 2-Cl-4-CN—Ph, R¹ is Br, R² is Me. |
| 261A | Q¹ is 2-Cl-4-CN—Ph, R¹ is Cl, R² is H. |
| 262A | Q¹ is 2-Cl-4-CN—Ph, R¹ is Cl, R² is Me. |
| 263A | Q¹ is 2-Cl-4-CN—Ph, R¹ is Me, R² is H. |
| 264A | Q¹ is 2-Cl-4-CN—Ph, R¹ is Me, R² is Br. |
| 265A | Q¹ is 2-Cl-4-CN—Ph, R¹ is Me, R² is Cl. |
| 266A | Q¹ is 2-Cl-4-CN—Ph, R¹ is Me, R² is Me. |
| 267A | Q¹ is 6-Cl-3-pyridinyl, R¹ is H, R² is Br. |
| 268A | Q¹ is 6-Cl-3-pyridinyl, R¹ is H, R² is Cl. |
| 269A | Q¹ is 6-Cl-3-pyridinyl, R¹ is H, R² is Me. |
| 270A | Q¹ is 6-Cl-3-pyridinyl, R¹ is Br, R² is H. |
| 271A | Q¹ is 6-Cl-3-pyridinyl, R¹ is Br, R² is Br. |
| 272A | Q¹ is 6-Cl-3-pyridinyl, R¹ is Br, R² is Cl. |
| 273A | Q¹ is 6-Cl-3-pyridinyl, R¹ is Br, R² is Me. |
| 274A | Q¹ is 6-Cl-3-pyridinyl, R¹ is Cl, R² is H. |
| 275A | Q¹ is 6-Cl-3-pyridinyl, R¹ is Cl, R² is Br. |
| 276A | Q¹ is 6-Cl-3-pyridinyl, R¹ is Cl, R² is Cl. |
| 277A | Q¹ is 6-Cl-3-pyridinyl, R¹ is Cl, R² is Me. |
| 278A | Q¹ is 6-Cl-3-pyridinyl, R¹ is Me, R² is H. |
| 279A | Q¹ is 6-Cl-3-pyridinyl, R¹ is Me, R² is Br. |
| 280A | Q¹ is 6-Cl-3-pyridinyl, R¹ is Me, R² is Cl. |
| 281A | Q¹ is 6-Cl-3-pyridinyl, R¹ is Me, R² is Me. |
| 282A | Q¹ is 2,6-di-F-4-CN—Ph, R¹ is Me, R² is Me. |
| 283A | Q¹ is 2,6-di-F-4-CN—Ph, R¹ is Cl, R² is Me. |
| 284A | Q¹ is 2,6-di-F-4-CN—Ph, R¹ is Me, R² is H. |
| 285A | Q¹ is 2,6-di-F-4-CN—Ph, R¹ is Me, R² is Br. |
| 286A | Q¹ is 2,6-di-F-4-CN—Ph, R¹ is Me, R² is Cl. |
| 287A | Q¹ is 2,6-di-F-4-CN—Ph, R¹ is Cl, R² is H. |
| 288A | Q¹ is 2,6-di-F-4-CN—Ph, R¹ is Cl, R² is Br. |
| 289A | Q¹ is 2,6-di-F-4-CN—Ph, R¹ is Cl, R² is Cl |
| 290A | Q¹ is 2,6-di-F-4-CN—Ph, R¹ is H, R² is Br. |
| 291A | Q¹ is 2,6-di-F-4-CN—Ph, R¹ is H, R² is Cl. |
| 292A | Q¹ is 2,6-di-F-4-CN—Ph, R¹ is H, R² is Me. |
| 293A | Q¹ is 2,6-di-F-4-CN—Ph, R¹ is Br, R² is H. |
| 294A | Q¹ is 2,6-diF-4-CN—Ph, R¹ is Br, R² is Br. |
| 295A | Q¹ is 2,6-di-F-4-CN—Ph, R¹ is Br, R² is Cl. |
| 296A | Q¹ is 2,6-di-F-4-CN—Ph, R¹ is Br, R² is Me. |
| 297A | Q¹ is 2-Cl-4-Me—Ph, R¹ is H, R² is Br. |
| 298A | Q¹ is 2-Cl-4-Me—Ph, R¹ is H, R² is Cl. |
| 299A | Q¹ is 2-Cl-4-Me—Ph, R¹ is H, R² is Me. |
| 300A | Q¹ is 2-Cl-4-Me—Ph, R¹ is Br, R² is H. |
| 301A | Q¹ is 2-Cl-4-Me—Ph, R¹ is Br, R² is Br. |
| 302A | Q¹ is 2-Cl-4-Me—Ph, R¹ is Br, R² is Cl. |
| 303A | Q¹ is 2-Cl-4-Me—Ph, R¹ is Br, R² is Me. |
| 304A | Q¹ is 2-Cl-4-Me—Ph, R¹ is Cl, R² is H. |
| 305A | Q¹ is 2-Cl-4-Me—Ph, R¹ is Cl, R² is Br. |
| 306A | Q¹ is 2-Cl-4-Me—Ph, R¹ is Cl, R² is Cl. |
| 307A | Q¹ is 2-Cl-4-Me—Ph, R¹ is Cl, R² is Me. |
| 308A | Q¹ is 2-Cl-4-Me—Ph, R¹ is Me, R² is H. |
| 309A | Q¹ is 2-Cl-4-Me—Ph, R¹ is Me, R² is Br. |
| 310A | Q¹ is 2-Cl-4-Me—Ph, R¹ is Me, R² is Cl. |
| 311A | Q¹ is 2-Cl-4-Me—Ph, R¹ is Me, R² is Me. |
| 312A | Q¹ is 2-Me-4-Cl—Ph, R¹ is H, R² is Br. |
| 313A | Q¹ is 2-Me-4-Cl—Ph, R¹ is H, R² is Cl. |
| 314A | Q¹ is 2-Me-4-Cl—Ph, R¹ is H, R² is Me. |
| 315A | Q¹ is 2-Me-4-Cl—Ph, R¹ is Br, R² is H. |
| 316A | Q¹ is 2-Me-4-Cl—Ph, R¹ is Br, R² is Br. |
| 317A | Q¹ is 2-Me-4-Cl—Ph, R¹ is Br, R² is Cl. |
| 318A | Q¹ is 2-Me-4-Cl—Ph, R¹ is Br, R² is Me. |
| 319A | Q¹ is 2-Me-4-Cl—Ph, R¹ is Cl, R² is H. |
| 320A | Q¹ is 2-Me-4-Cl—Ph, R¹ is Cl, R² is Br. |
| 321A | Q¹ is 2-Me-4-Cl—Ph, R¹ is Cl, R² is Cl. |
| 322A | Q¹ is 2-Me-4-Cl—Ph, R¹ is Cl, R² is Me. |
| 323A | Q¹ is 2-Me-4-Cl—Ph, R¹ is Me, R² is H. |
| 324A | Q¹ is 2-Me-4-Cl—Ph, R¹ is Me, R² is Br. |
| 325A | Q¹ is 2-Me-4-Cl—Ph, R¹ is Me, R² is Cl. |
| 326A | Q¹ is 2-Me-4-Cl—Ph, R¹ is Me, R² is Me. |
| 327A | Q¹ is 2-Br-4-MeO—Ph, R¹ is H, R² is Br. |
| 328A | Q¹ is 2-Br-4-MeO—Ph, R¹ is H, R² is Cl. |
| 329A | Q¹ is 2-Br-4-MeO—Ph, R¹ is H, R² is Me. |
| 330A | Q¹ is 2-Br-4-MeO—Ph, R¹ is Br, R² is H. |
| 331A | Q¹ is 2-Br-4-MeO—Ph, R¹ is Br, R² is Br. |
| 332A | Q¹ is 2-Br-4-MeO—Ph, R¹ is Br, R² is Cl. |
| 333A | Q¹ is 2-Br-4-MeO—Ph, R¹ is Br, R² is Me. |
| 334A | Q¹ is 2-Br-4-MeO—Ph, R¹ is Cl, R² is H. |
| 335A | Q¹ is 2-Br-4-MeO—Ph, R¹ is Cl, R² is Br. |
| 336A | Q¹ is 2-Br-4-MeO—Ph, R¹ is Cl, R² is Cl. |
| 337A | Q¹ is 2-Br-4-MeO—Ph, R¹ is Cl, R² is Me. |
| 338A | Q¹ is 2-Br-4-MeO—Ph, R¹ is Me, R² is H. |
| 339A | Q¹ is 2-Br-4-MeO—Ph, R¹ is Me, R² is Br. |
| 340A | Q¹ is 2-Br-4-MeO—Ph, R¹ is Me, R² is Cl. |
| 341A | Q¹ is 2-Br-4-MeO—Ph, R¹ is Me, R² is Me. |
| 342A | Q¹ is 2-Me-4-MeO—Ph, R¹ is H, R² is Br. |
| 343A | Q¹ is 2-Me-4-MeO—Ph, R¹ is H, R² is Cl. |
| 344A | Q¹ is 2-Me-4-MeO—Ph, R¹ is H, R² is Me. |
| 345A | Q¹ is 2-Me-4-MeO—Ph, R¹ is Br, R² is H. |
| 346A | Q¹ is 2-Me-4-MeO—Ph, R¹ is Br, R² is Br. |
| 347A | Q¹ is 2-Me-4-MeO—Ph, R¹ is Br, R² is Cl. |
| 348A | Q¹ is 2-Me-4-MeO—Ph, R¹ is Br, R² is Me. |
| 349A | Q¹ is 2-Me-4-MeO—Ph, R¹ is Cl, R² is H. |
| 350A | Q¹ is 2-Me-4-MeO—Ph, R¹ is Cl, R² is Br. |
| 351A | Q¹ is 2-Me-4-MeO—Ph, R¹ is Cl, R² is Cl. |
| 352A | Q¹ is 2-Me-4-MeO—Ph, R¹ is Cl, R² is Me. |
| 353A | Q¹ is 2-Me-4-MeO—Ph, R¹ is Me, R² is H. |
| 354A | Q¹ is 2-Me-4-MeO—Ph, R¹ is Me, R² is Br. |
| 355A | Q¹ is 2-Me-4-MeO—Ph, R¹ is Me, R² is Cl. |
| 356A | Q¹ is 2-Me-4-MeO—Ph, R¹ is Me, R² is Me. |

TABLE 2 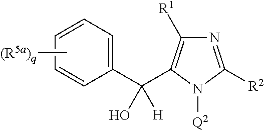
(R^{5a})ₚ is 4-MeNH(CH₂)₃O, R¹ is H, R² is Cl.

| Q² | Q² | Q² | Q² | Q² |
|---|---|---|---|---|
| 2-Br—Ph | 2-Br-4-MeO—Ph | 2-F-4-MeO—Ph | 2,4-di-Cl-6-F—Ph | 2-Br-4-Cl-6-F—Ph |
| 2-Cl—Ph | 2-Br-4-EtO—Ph | 2-F-4-EtO—Ph | 2,6-di-Cl-4-CN—Ph | 2-Br-4-F-6-Cl—Ph |
| 2-F—Ph | 2-Cl-4-Br—Ph | 2-I-4-F—Ph | 2,6-di-Cl-4-F—Ph | 2-Cl-4-Br-6-F—Ph |
| 2-I—Ph | 2-Cl-4-CN—Ph | 2-I-6-F—Ph | 2,6-di-Cl-4-MeO—Ph | 2-Br-3-pyridinyl |
| 2-CF₃—Ph | 2-Cl-4-F—Ph | 2-CF₃-4-F—Ph | 2,6-di-F-4-Br—Ph | 2-Cl-3-pyridinyl |
| 2,4-di-Cl—Ph | 2-Cl-6-F—Ph | 2-CF₃-6-F—Ph | 2,6-di-F-4-Cl—Ph | 2-Me-3-pyridinyl |
| 2,6-di-Cl—Ph | 2-Cl-4-I—Ph | 2-Me-4-F—Ph | 2,6-di-F-4-CN—Ph | 2,4-di-Cl-3-pyridinyl |
| 2,4-di-F—Ph | 2-Cl-4-MeO—Ph | 2,4,6-tri-Cl—Ph | 2,6-di-F-4-I—Ph | 2,6-di-Cl-3-pyridinyl |
| 2,6-di-F—Ph | 2-Cl-4-EtO—Ph | 2,3,5-tri-F—Ph | 2,6-di-F-4-MeO—Ph | 3,5-di-Cl-2-pyridinyl |
| 2-Br-4-Cl—Ph | 2-F-4-Br—Ph | 2,3,6-tri-F—Ph | 2,6-di-F-4-EtO—Ph | 3,5-di-F-2-pyridinyl |
| 2-Br-4-CN—Ph | 2-F-4-Cl—Ph | 2,4,5-tri-F—Ph | 2-Br-4,6-di-F—Ph | 2-Cl-6-MeO-3-pyridinyl |
| 2-Br-4-F—Ph | 2-F-4-CN—Ph | 2,4,6-tri-F—Ph | 2-Cl-4,6-di-F—Ph | 1,3-di-Me-1H-4-pyrazol-5-yl |
| 2-Br-6-F—Ph | 2-F-4-I—Ph | 2,4-di-Br-6-F—Ph | 2-I-4,6-di-F—Ph | 2-Br-3-thienyl |

The present disclosure also includes Tables 1B through 44B, each of which is constructed the same as Table 2 above, except that the row heading in Table 2 (i.e. “(R^{5a})ₚ is 4-MeNH(CH₂)₃O, R¹ is H, R² is Cl.”) is replaced with the respective row heading shown below. For Example, in Table 1B the row heading is “(R^{5a})ₚ is 4-MeNH(CH₂)₃O, R¹ is Br, R² is Cl.”, and Q² is as defined in Table 2 above. Thus, the first entry in Table 1B specifically discloses 4-bromo-1-(2-bromophenyl)-2-chloro-α-[4-[3-(methylamino)-propoxy]phenyl]-1H-imidazole-5-methanol. Tables 2B through 44B are constructed similarly.

| Table | Row Heading |
|---|---|
| 1B | (R^{5a})ₚ is 4-MeNH(CH₂)₃O, R¹ is Br, R² is Cl. |
| 2B | (R^{5a})ₚ is 4-MeNH(CH₂)₃O, R¹ is Me, R² is Cl. |
| 3B | (R^{5a})ₚ is 2-F-4-MeNH(CH₂)₃O, R¹ is Me, R² is Br. |
| 4B | (R^{5a})ₚ is 2-F-4-MeNH(CH₂)₃O, R¹ is Cl, R² is Cl. |
| 5B | (R^{5a})ₚ is 2-F-4-MeNH(CH₂)₃O, R¹ is Cl, R² is Br. |
| 6B | (R^{5a})ₚ is 2,6-di-F-4-MeNH(CH₂)₃O, R¹ is H, R² is Cl. |
| 7B | (R^{5a})ₚ is 2,6-di-F-4-MeNH(CH₂)₃O, R¹ is Cl, R² is Br. |
| 8B | (R^{5a})ₚ is 2,6-di-F-4-MeNH(CH₂)₃O, R¹ is Br, R² is Me. |
| 9B | (R^{5a})ₚ is 2,6-di-F-4-MeNH(CH₂)₃O, R¹ is Cl, R² is Cl. |
| 10B | (R^{5a})ₚ is 2,6-di-F-4-MeNH(CH₂)₃O, R¹ is Cl, R² is Me. |
| 11B | (R^{5a})ₚ is 2-Cl-6-F-4-MeNH(CH₂)₃O, R¹ is H, R² is Me. |
| 12B | (R^{5a})ₚ is 2-Cl-6-F-4-MeNH(CH₂)₃O, R¹ is Cl, R² is Me. |
| 13B | (R^{5a})ₚ is 2-Cl-6-F-4-MeNH(CH₂)₃O, R¹ is Br, R² is Me. |
| 14B | (R^{5a})ₚ is 4-Me₂N(CH₂)₃O, R¹ is Me, R² is Cl. |
| 15B | (R^{5a})ₚ is 4-Me₂N(CH₂)₃O, R¹ is Br, R² is Cl. |
| 16B | (R^{5a})ₚ is 4-Me₂N(CH₂)₃O, R¹ is Cl, R² is Cl. |
| 17B | (R^{5a})ₚ is 2-F-4-Me₂N(CH₂)₃O, R¹ is Cl, R² is Cl. |
| 18B | (R^{5a})ₚ is 2-F-4-Me₂N(CH₂)₃O, R¹ is Cl, R² is Br. |
| 19B | (R^{5a})ₚ is 2-F-4-Me₂N(CH₂)₃O, R¹ is Me, R² is Cl. |
| 20B | (R^{5a})ₚ is 2,6-di-F-4-Me₂N(CH₂)₃O, R¹ is H, R² is Cl. |
| 21B | (R^{5a})ₚ is 2,6-di-F-4-Me₂N(CH₂)₃O, R¹ is Cl, R² is Cl. |
| 22B | (R^{5a})ₚ is 2,6-di-F-4-Me₂N(CH₂)₃O, R¹ is Cl, R² is Me. |
| 23B | (R^{5a})ₚ is 2-Cl-6-F-4-MeO(CH₂)₃O, R¹ is Cl, R² is Cl. |
| 24B | (R^{5a})ₚ is 2-Cl-6-F-4-MeO(CH₂)₃O, R¹ is Cl, R² is Br. |
| 25B | (R^{5a})ₚ is 2-Cl-6-F-4-MeO(CH₂)₃O, R¹ is Cl, R² is Me. |
| 26B | (R^{5a})ₚ is 2-F-4-MeO(CH₂)₃O, R¹ is Me, R² is Me. |
| 27B | (R^{5a})ₚ is 2-F-4-MeO(CH₂)₃O, R¹ is H, R² is Cl. |
| 28B | (R^{5a})ₚ is 2-F-4-MeO(CH₂)₃O, R¹ is Br, R² is Cl. |
| 29B | (R^{5a})ₚ is 2-Cl-4-MeO(CH₂)₃O, R¹ is Br, R² is Cl. |
| 30B | (R^{5a})ₚ is 2-Cl-4-MeO(CH₂)₃O, R¹ is Me, R² is Cl. |
| 31B | (R^{5a})ₚ is 2-Cl-4-MeO(CH₂)₃O, R¹ is Cl, R² is Cl. |
| 32B | (R^{5a})ₚ is 2,6-di-F-4-MeO(CH₂)₃O, R¹ is Br, R² is Cl. |
| 33B | (R^{5a})ₚ is 2,6-di-F-4-MeO(CH₂)₃O, R¹ is Me, R² is Cl. |
| 34B | (R^{5a})ₚ is 2,6-di-F-4-MeO(CH₂)₃O, R¹ is Cl, R² is Cl. |
| 35B | (R^{5a})ₚ is 2-Cl-6-F-4-MeO(CH₂)₃O, R¹ is Me, R² is Cl. |
| 36B | (R^{5a})ₚ is 2-Cl-6-F-4-MeO(CH₂)₃O, R¹ is H, R² is Cl. |
| 37B | (R^{5a})ₚ is 2-Cl-6-F-4-MeO(CH₂)₃O, R¹ is Br, R² is Cl. |
| 38B | (R^{5a})ₚ is 2,6-di-F-3-MeNH(CH₂)₃O, R¹ is H, R² is Cl. |
| 39B | (R^{5a})ₚ is 2,6-di-F-3-MeNH(CH₂)₃O, R¹ is Br, R² is Cl. |
| 40B | (R^{5a})ₚ is 2,6-di-F-3-MeNH(CH₂)₃O, R¹ is Me, R² is Cl. |
| 41B | (R^{5a})ₚ is 2,6-di-F-3-MeNH(CH₂)₃O, R¹ is Cl, R² is Cl. |
| 42B | (R^{5a})ₚ is 2,6-F-3-Me₂N(CH₂)₃O, R¹ is Br, R² is Cl. |
| 43B | (R^{5a})ₚ is 2,6-F-3-Me₂N(CH2)₃O, R¹ is Cl, R² is Br. |
| 44B | (R^{5a})ₚ is 2,6-F-3-Me₂N(CH2)₃O, R¹ is Cl, R² is Me. |

TABLE 3

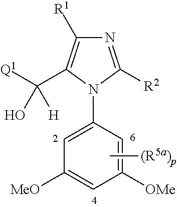

| Q¹ | R¹ | R² | (R^{5a})ₚ |
|---|---|---|---|
| 2,4-di-F—Ph | Me | Me | H |
| 2-Cl-4-F—Ph | Me | Me | 2-Cl |
| 2-Me-4-F—Ph | Me | Me | 2-Br |
| 2-Cl-4-F—Ph | Me | Br | H |
| 2,4-di-F—Ph | Me | Br | 2-Cl |
| 2-Cl-4-F—Ph | Me | Br | 2-Br |
| 2-Cl-4-F—Ph | Me | Cl | H |
| 2-Me-4-F—Ph | Me | Cl | 2-Cl |
| 2,4-di-F—Ph | Me | Cl | 2-Br |
| 2,4-di-F—Ph | Br | Br | H |
| 2-Cl-4-F—Ph | Br | Cl | 2-Cl |
| 2-Me-4-F—Ph | Br | Me | 2-Br |
| 2,4-di-F—Ph | Cl | Br | H |
| 2-Cl-4-F—Ph | Cl | Cl | 2-Cl |
| 2-Cl-4-F—Ph | Cl | Me | 2-Br |

As disclosed in Scheme 2 above, compounds of Formula 3 are useful intermediates for the preparation of compounds of Formula 1a (i.e. Formula 1 wherein R³ is —OR⁶ and R⁶ is H). The present invention includes but is not limited to the exemplary species of the compounds Formula 3 disclosed in Table 4.

TABLE 4 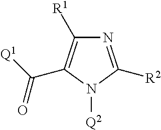
Q¹ is 2,4-di-F—Ph, R¹ is Me, R² is Cl.

| Q² | Q² | Q² | Q² | Q² |
|---|---|---|---|---|
| 2,6-di-F—Ph | 2-Cl-4-F | 2-Cl-6-F | 2-Br-4-F | 2-Br-6-F |
| 2-Cl-4,6-diF | 2-Br-4,6-di-F | 2,4,6-tri-F | 2-F-4-Me | 2-Cl-4-Me |

The present disclosure also includes exemplary species of the compounds Formula 3 disclosed in Tables 1C through 71C, each of which is constructed the same as Table 4 above, except that the row heading in Table 4 (i.e. “Q¹ is 2,4-di-F-Ph, R¹ is Me, R² is Cl”) is replaced with the respective row heading shown below. For Example, in Table 1C the row heading is “Q¹ is 2,4-di-F-Ph, R¹ is Me, R² is Br”, and Q² is as defined in Table 4 above. Thus, the first entry in Table 1C specifically discloses [2-bromo-1-(2,6-difluorophenyl)-4-methyl-1H-imidazol-5-yl](2,4-difluorophenyl)methanone. Tables 2C through 71C are constructed similarly.

| Table | Row Heading |
|---|---|
| 1C | Q¹ is 2,4-di-F—Ph, R¹ is Me, R² is Br. |
| 2C | Q¹ is 2,4-di-F—Ph, R¹ is Cl, R² is Me. |
| 3C | Q¹ is 2,4-di-F—Ph, R¹ is Cl, R² is Cl. |
| 4C | Q¹ is 2,4-di-F—Ph, R¹ is Cl, R² is Br. |
| 5C | Q¹ is 2,4-di-F—Ph, R¹ is Br, R² is Me. |
| 6C | Q¹ is 2,4-di-F—Ph, R¹ is Br, R² is Cl. |
| 7C | Q¹ is 2,4-di-F—Ph, R¹ is Br, R² is Br. |
| 8C | Q¹ is 2-F-4-MeO—Ph, R¹ is Me, R² is Cl. |
| 9C | Q¹ is 2-F-4-MeO—Ph, R¹ is Me, R² is Br. |
| 10C | Q¹ is 2-F-4-MeO—Ph, R¹ is Cl, R² is Me. |
| 11C | Q¹ is 2-F-4-MeO—Ph, R¹ is Cl, R² is Cl. |
| 12C | Q¹ is 2-F-4-MeO—Ph, R¹ is Cl, R² is Br. |
| 13C | Q¹ is 2-F-4-MeO—Ph, R¹ is Br, R² is Me. |
| 14C | Q¹ is 2-F-4-MeO—Ph, R¹ is Br, R² is Cl. |
| 15C | Q¹ is 2-F-4-MeO—Ph, R¹ is Br, R² is Br. |
| 16C | Q¹ is 2-Cl-4-F—Ph, R¹ is Me, R² is Cl. |
| 17C | Q¹ is 2-Cl-4-F—Ph, R¹ is Me, R² is Br. |
| 18C | Q¹ is 2-Cl-4-F—Ph, R¹ is Cl, R² is Me. |
| 19C | Q¹ is 2-Cl-4-F—Ph, R¹ is Cl, R² is Cl. |
| 20C | Q¹ is 2-Cl-4-F—Ph, R¹ is Cl, R² is Br. |
| 21C | Q¹ is 2-Cl-4-F—Ph, R¹ is Br, R² is Me. |
| 22C | Q¹ is 2-Cl-4-F—Ph, R¹ is Br, R² is Cl. |
| 23C | Q¹ is 2-Cl-4-F—Ph, R¹ is Br, R² is Br. |
| 24C | Q¹ is 2-Cl-4-MeO—Ph, R¹ is Me, R² is Cl. |
| 25C | Q¹ is 2-Cl-4-MeO—Ph, R¹ is Me, R² is Br. |
| 26C | Q¹ is 2-Cl-4-MeO—Ph, R¹ is Cl, R² is Me. |
| 27C | Q¹ is 2-Cl-4-MeO—Ph R¹ is Cl, R² is Cl. |
| 28C | Q¹ is 2-Cl-4-MeO—Ph, R¹ is Cl, R² is Br. |
| 29C | Q¹ is 2-Cl-4-MeO—Ph, R¹ is Br, R² is Me. |
| 30C | Q¹ is 2-Cl-4-MeO—Ph, R¹ is Br, R² is Cl. |
| 31C | Q¹ is 2-Cl-4-MeO—Ph, R¹ is Br, R² is Br. |
| 32C | Q¹ is 2-Br-4-F—Ph, R¹ is Me, R² is Cl. |
| 33C | Q¹ is 2-Br-4-F—Ph, R¹ is Me, R² is Br. |
| 34C | Q¹ is 2-Br-4-F—Ph, R¹ is Cl, R² is Me. |
| 35C | Q¹ is 2-Br-4-F—Ph, R¹ is Cl, R² is Cl. |
| 36C | Q¹ is 2-Br-4-F—Ph, R¹ is Cl, R² is Br. |
| 37C | Q¹ is 2-Br-4-F—Ph, R¹ is Br, R² is Me. |
| 38C | Q¹ is 2-Br-4-F—Ph, R¹ is Br, R² is Cl. |
| 39C | Q¹ is 2-Br-4-F—Ph, R¹ is Br, R² is Br. |
| 40C | Q¹ is 2-Br-4-MeO—Ph, R¹ is Me, R² is Cl. |
| 41C | Q¹ is 2-Br-4-MeO—Ph, R¹ is Me, R² is Br. |
| 42C | Q¹ is 2-Br-4-MeO—Ph, R¹ is Cl, R² is Me. |
| 43C | Q¹ is 2-Br-4-MeO—Ph, R¹ is Cl, R² is Cl. |
| 44C | Q¹ is 2-Br-4-MeO—Ph, R¹ is Cl, R² is Br. |
| 45C | Q¹ is 2-Br-4-MeO—Ph, R¹ is Br, R² is Me. |
| 46C | Q¹ is 2-Br-4-MeO—Ph, R¹ is Br, R² is Cl. |
| 47C | Q¹ is 2-Br-4-MeO—Ph, R¹ is Br, R² is Br. |
| 48C | Q¹ is 2-Me-4-F—Ph, R¹ is Me, R² is Cl. |
| 49C | Q¹ is 2-Me-4-F—Ph, R¹ is Me, R² is Br. |
| 50C | Q¹ is 2-Me-4-F—Ph, R¹ is Cl, R² is Me. |
| 51C | Q¹ is 2-Me-4-F—Ph, R¹ is Cl, R² is Cl. |
| 52C | Q¹ is 2-Me-4-F—Ph, R¹ is Cl, R² is Br. |
| 53C | Q¹ is 2-Me-4-F—Ph, R¹ is Br, R² is Me. |
| 54C | Q¹ is 2-Me-4-F—Ph, R¹ is Br, R² is Cl. |
| 55C | Q¹ is 2-Me-4-F—Ph, R¹ is Br, R² is Br. |
| 56C | Q¹ is 2-Me-4-Cl—Ph, R¹ is Me, R² is Cl. |
| 57C | Q¹ is 2-Me-4-Cl—Ph, R¹ is Me, R² is Br. |
| 58C | Q¹ is 2-Me-4-Cl—Ph, R¹ is Cl, R² is Me. |
| 59C | Q¹ is 2-Me-4-Cl—Ph, R¹ is Cl, R² is Cl. |
| 60C | Q¹ is 2-Me-4-Cl—Ph, R¹ is Cl, R² is Br. |
| 61C | Q¹ is 2-Me-4-Cl—Ph, R¹ is Br, R² is Me. |
| 62C | Q¹ is 2-Me-4-Cl—Ph, R¹ is Br, R² is Cl. |
| 63C | Q¹ is 2-Me-4-Cl—Ph, R¹ is Br, R² is Br. |
| 64C | Q¹ is 2-Me-4-MeO—Ph, R¹ is Me, R² is Cl. |
| 65C | Q¹ is 2-Me-4-MeO—Ph, R¹ is Me, R² is Br. |
| 66C | Q¹ is 2-Me-4-MeO—Ph, R¹ is Cl, R² is Me. |
| 67C | Q¹ is 2-Me-4-MeO—Ph, R¹ is Cl, R² is Cl. |
| 68C | Q¹ is 2-Me-4-MeO—Ph, R¹ is Cl, R² is Br. |
| 69C | Q¹ is 2-Me-4-MeO—Ph, R¹ is Br, R² is Me. |
| 70C | Q¹ is 2-Me-4-MeO—Ph, R¹ is Br, R² is Cl. |
| 71C | Q¹ is 2-Me-4-MeO—Ph, R¹ is Br, R² is Br. |

As disclosed in Scheme 1 above, compounds of Formula 2 are useful intermediates for the preparation of compounds of Formula 1a (i.e. Formula 1 wherein R³ is —OR⁶ and R⁶ is H). The present invention includes but is not limited to the exemplary species of the compounds Formula 2 disclosed in Table 5.

TABLE 5

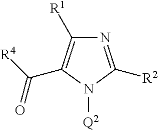

| | |
|---|---|
| Q² | Q² |

R¹ is Me, R² is Cl, R⁴ is H.

| | |
|---|---|
| 2,6-di-F—Ph | 2,4,6-tri-F |
| 2-Cl-4,6-di-F | 2-Br-4-F |
| 2-Cl-4-F | 2-F-4-Me |
| 2-Br-4,6-di-F | 2-Br-6-F |
| 2-Cl-6-F | 2-Cl-4-Me |

R¹ is H, R² is Me, R⁴ is H.

| | |
|---|---|
| 2,6-di-F—Ph | 2,4,6-tri-F |
| 2-Cl-4,6-di-F | 2-Br-4-F |
| 2-Cl-4-F | 2-F-4-Me |
| 2-Br-4,6-di-F | 2-Br-6-F |
| 2-Cl-6-F | 2-Cl-4-Me |

R¹ is Me, R² is Br, R⁴ is H.

| | |
|---|---|
| 2,6-di-F—Ph | 2,4,6-tri-F |
| 2-Cl-4,6-di-F | 2-Br-4-F |
| 2-Cl-4-F | 2-F-4-Me |
| 2-Br-4,6-di-F | 2-Br-6-F |
| 2-Cl-6-F | 2-Cl-4-Me |

### Formulation/Utility

A compound of this invention will generally be used as a fungicidal active ingredient in a composition, i.e. formulation, with at least one additional component selected from the group consisting of surfactants, solid diluents and liquid diluents, which serve as a carrier.

The formulation or composition ingredients are selected to be consistent with the physical properties of the active ingredient, mode of application and environmental factors such as soil type, moisture and temperature.

Useful formulations include both liquid and solid compositions. Liquid compositions include solutions (including emulsifiable concentrates), suspensions, emulsions (including microemulsions and/or suspoemulsions) and the like, which optionally can be thickened into gels. The general types of aqueous liquid compositions are soluble concentrate, suspension concentrate, capsule suspension, concentrated emulsion, microemulsion and suspo-emulsion. The general types of nonaqueous liquid compositions are emulsifiable concentrate, microemulsifiable concentrate, dispersible concentrate and oil dispersion.

The general types of solid compositions are dusts, powders, granules, pellets, prills, pastilles, tablets, filled films (including seed coatings) and the like, which can be water-dispersible (“wettable”) or water-soluble. Films and coatings formed from film-forming solutions or flowable suspensions are particularly useful for seed treatment. Active ingredient can be (micro)encapsulated and further formed into a suspension or solid formulation; alternatively the entire formulation of active ingredient can be encapsulated (or “overcoated”). Encapsulation can control or delay release of the active ingredient. An emulsifiable granule combines the advantages of both an emulsifiable concentrate formulation and a dry granular formulation. High-strength compositions are primarily used as intermediates for further formulation.

Sprayable formulations are typically extended in a suitable medium before spraying. Such liquid and solid formulations are formulated to be readily diluted in the spray medium, usually water. Spray volumes can range from about one to several thousand liters per hectare, but more typically are in the range from about ten to several hundred liters per hectare. Sprayable formulations can be tank mixed with water or another suitable medium for foliar treatment by aerial or ground application, or for application to the growing medium of the plant. Liquid and dry formulations can be metered directly into drip irrigation systems or metered into the furrow during planting. Liquid and solid formulations can be applied onto seeds of crops and other desirable vegetation as seed treatments before planting to protect developing roots and other subterranean plant parts and/or foliage through systemic uptake.

The formulations will typically contain effective amounts of active ingredient, diluent and surfactant within the following approximate ranges which add up to 100 percent by weight.

| | Weight Percent | | |
|---|---|---|---|
| | Active | | |
| | Ingredient | Diluent | Surfactant |
| Water-Dispersible and Water- | 0.001-90 | 0-99.999 | 0-15 |
| soluble Granules, Tablets and | | | |
| Powders | | | |
| Oil Dispersions, Suspensions, | 1-50 | 40-99 | 0-50 |
| Emulsions, Solutions | | | |
| (including Emulsifiable | | | |
| Concentrates) | | | |
| Dusts | 1-25 | 70-99 | 0-5 |
| Granules and Pellets | 0.001-95 | 5-99.999 | 0-15 |
| High Strength Compositions | 90-99 | 0-10 | 0-2 |

Solid diluents include, for example, clays such as bentonite, montmorillonite, attapulgite and kaolin, gypsum, cellulose, titanium dioxide, zinc oxide, starch, dextrin, sugars (e.g., lactose, sucrose), silica, talc, mica, diatomaceous earth, urea, calcium carbonate, sodium carbonate and bicarbonate, and sodium sulfate. Typical solid diluents are described in Watkins et al., Handbook of Insecticide Dust Diluents and Carriers, 2nd Ed., Dorland Books, Caldwell, N.J.

Liquid diluents include, for example, water, N,N-dimethylalkanamides (e.g., N,N-dimethylformamide), limonene, dimethyl sulfoxide, N-alkylpyrrolidones (e.g., N-methylpyrrolidinone), ethylene glycol, triethylene glycol, propylene glycol, dipropylene glycol, polypropylene glycol, propylene carbonate, butylene carbonate, paraffins (e.g., white mineral oils, normal paraffins, isoparaffins), alkylbenzenes, alkylnaphthalenes, glycerine, glycerol triacetate, sorbitol, aromatic hydrocarbons, dearomatized aliphatics, alkylbenzenes, alkylnaphthalenes, ketones such as cyclohexanone, 2-heptanone, isophorone and 4-hydroxy-4-methyl-2-pentanone, acetates such as isoamyl acetate, hexyl acetate, heptyl acetate, octyl acetate, nonyl acetate, tridecyl acetate and isobornyl acetate, other esters such as alkylated lactate esters, dibasic esters and γ-butyrolactone, and alcohols, which can be linear, branched, saturated or unsaturated, such as methanol, ethanol, n-propanol, isopropyl alcohol, n-butanol, isobutyl alcohol, n-hexanol, 2-ethylhexanol, n-octanol, decanol, isodecyl alcohol, isooctadecanol, cetyl alcohol, lauryl alcohol, tridecyl alcohol, oleyl alcohol, cyclohexanol, tetrahydrofurfuryl alcohol, diacetone alcohol and benzyl alcohol. Liquid diluents also include glycerol esters of saturated and unsaturated fatty acids (typically C₆-C₂₂), such as plant seed and fruit oils (e.g., oils of olive, castor, linseed, sesame, corn (maize), peanut, sunflower, grapeseed, safflower, cottonseed, soybean, rapeseed, coconut and palm kernel), animal-sourced fats (e.g., beef tallow, pork tallow, lard, cod liver oil, fish oil), and mixtures thereof. Liquid diluents also include alkylated fatty acids (e.g., methylated, ethylated, butylated) wherein the fatty acids may be obtained by hydrolysis of glycerol esters from plant and animal sources, and can be purified by distillation. Typical liquid diluents are described in Marsden, Solvents Guide, 2nd Ed., Interscience, New York, 1950.

The solid and liquid compositions of the present invention often include one or more surfactants. When added to a liquid, surfactants (also known as “surface-active agents”) generally modify, most often reduce, the surface tension of the liquid. Depending on the nature of the hydrophilic and lipophilic groups in a surfactant molecule, surfactants can be useful as wetting agents, dispersants, emulsifiers or defoaming agents.

Surfactants can be classified as nonionic, anionic or cationic. Nonionic surfactants useful for the present compositions include, but are not limited to: alcohol alkoxylates such as alcohol alkoxylates based on natural and synthetic alcohols (which may be branched or linear) and prepared from the alcohols and ethylene oxide, propylene oxide, butylene oxide or mixtures thereof; amine ethoxylates, alkanolamides and ethoxylated alkanolamides; alkoxylated triglycerides such as ethoxylated soybean, castor and rapeseed oils; alkylphenol alkoxylates such as octylphenol ethoxylates, nonylphenol ethoxylates, dinonyl phenol ethoxylates and dodecyl phenol ethoxylates (prepared from the phenols and ethylene oxide, propylene oxide, butylene oxide or mixtures thereof); block polymers prepared from ethylene oxide or propylene oxide and reverse block polymers where the terminal blocks are prepared from propylene oxide; ethoxylated fatty acids; ethoxylated fatty esters and oils; ethoxylated methyl esters; ethoxylated tristyrylphenol (including those prepared from ethylene oxide, propylene oxide, butylene oxide or mixtures thereof); fatty acid esters, glycerol esters, lanolin-based derivatives, polyethoxylate esters such as polyethoxylated sorbitan fatty acid esters, polyethoxylated sorbitol fatty acid esters and polyethoxylated glycerol fatty acid esters; other sorbitan derivatives such as sorbitan esters; polymeric surfactants such as random copolymers, block copolymers, alkyd peg (polyethylene glycol) resins, graft or comb polymers and star polymers; polyethylene glycols (pegs); polyethylene glycol fatty acid esters; silicone-based surfactants; and sugar-derivatives such as sucrose esters, alkyl polyglycosides and alkyl polysaccharides.

Useful anionic surfactants include, but are not limited to: alkylaryl sulfonic acids and their salts; carboxylated alcohol or alkylphenol ethoxylates; diphenyl sulfonate derivatives; lignin and lignin derivatives such as lignosulfonates; maleic or succinic acids or their anhydrides; olefin sulfonates; phosphate esters such as phosphate esters of alcohol alkoxylates, phosphate esters of alkylphenol alkoxylates and phosphate esters of styryl phenol ethoxylates; protein-based surfactants; sarcosine derivatives; styryl phenol ether sulfate; sulfates and sulfonates of oils and fatty acids; sulfates and sulfonates of ethoxylated alkylphenols; sulfates of alcohols; sulfates of ethoxylated alcohols; sulfonates of amines and amides such as N,N-alkyltaurates; sulfonates of benzene, cumene, toluene, xylene, and dodecyl and tridecylbenzenes; sulfonates of condensed naphthalenes; sulfonates of naphthalene and alkyl naphthalene; sulfonates of fractionated petroleum; sulfosuccinamates; and sulfosuccinates and their derivatives such as dialkyl sulfosuccinate salts.

Useful cationic surfactants include, but are not limited to: amides and ethoxylated amides; amines such as N-alkyl propanediamines, tripropylenetriamines and dipropylenetetramines, and ethoxylated amines, ethoxylated diamines and propoxylated amines (prepared from the amines and ethylene oxide, propylene oxide, butylene oxide or mixtures thereof); amine salts such as amine acetates and diamine salts; quaternary ammonium salts such as quaternary salts, ethoxylated quaternary salts and diquaternary salts; and amine oxides such as alkyldimethylamine oxides and bis-(2-hydroxyethyl)-alkylamine oxides.

Also useful for the present compositions are mixtures of nonionic and anionic surfactants or mixtures of nonionic and cationic surfactants. Nonionic, anionic and cationic surfactants and their recommended uses are disclosed in a variety of published references including McCutcheon's Emulsifiers and Detergents, annual American and International Editions published by McCutcheon's Division, The Manufacturing Confectioner Publishing Co.; Sisely and Wood, Encyclopedia of Surface Active Agents, Chemical Publ. Co., Inc., New York, 1964; and A. S. Davidson and B. Milwidsky, Synthetic Detergents, Seventh Edition, John Wiley and Sons, New York, 1987.

Compositions of this invention may also contain formulation auxiliaries and additives, known to those skilled in the art as formulation aids (some of which may be considered to also function as solid diluents, liquid diluents or surfactants). Such formulation auxiliaries and additives may control: pH (buffers), foaming during processing (antifoams such polyorganosiloxanes), sedimentation of active ingredients (suspending agents), viscosity (thixotropic thickeners), in-container microbial growth (antimicrobials), product freezing (antifreezes), color (dyes/pigment dispersions), wash-off (film formers or stickers), evaporation (evaporation retardants), and other formulation attributes. Film formers include, for example, polyvinyl acetates, polyvinyl acetate copolymers, polyvinylpyrrolidone-vinyl acetate copolymer, polyvinyl alcohols, polyvinyl alcohol copolymers and waxes. Examples of formulation auxiliaries and additives include those listed in McCutcheon's Volume 2: Functional Materials, annual International and North American editions published by McCutcheon's Division, The Manufacturing Confectioner Publishing Co.; and PCT Publication WO 03/024222.

The compound of Formula 1 and any other active ingredients are typically incorporated into the present compositions by dissolving the active ingredient in a solvent or by grinding in a liquid or dry diluent. Solutions, including emulsifiable concentrates, can be prepared by simply mixing the ingredients. If the solvent of a liquid composition intended for use as an emulsifiable concentrate is water-immiscible, an emulsifier is typically added to emulsify the active-containing solvent upon dilution with water. Active ingredient slurries, with particle diameters of up to 2,000 μm can be wet milled using media mills to obtain particles with average diameters below 3 μm. Aqueous slurries can be made into finished suspension concentrates (see, for example, U.S. Pat. No. 3,060,084) or further processed by spray drying to form water-dispersible granules. Dry formulations usually require dry milling processes, which produce average particle diameters in the 2 to 10 μm range. Dusts and powders can be prepared by blending and usually grinding (such as with a hammer mill or fluid-energy mill). Granules and pellets can be prepared by spraying the active material upon preformed granular carriers or by agglomeration techniques. See Browning, “Agglomeration”, Chemical Engineering, Dec. 4, 1967, pp 147-48, Perry's Chemical Engineer's Handbook, 4th Ed., McGraw-Hill, New York, 1963, pages 8-57 and following, and WO 91/13546. Pellets can be prepared as described in U.S. Pat. No. 4,172,714. Water-dispersible and water-soluble granules can be prepared as taught in U.S. Pat. No. 4,144,050, U.S. Pat. No. 3,920,442 and DE 3,246,493. Tablets can be prepared as taught in U.S. Pat. No. 5,180,587, U.S. Pat. No. 5,232,701 and U.S. Pat. No. 5,208,030. Films can be prepared as taught in GB 2,095,558 and U.S. Pat. No. 3,299,566.

For further information regarding the art of formulation, see T. S. Woods, “The Formulator's Toolbox—Product Forms for Modern Agriculture” in Pesticide Chemistry and Bioscience, The Food—Environment Challenge, T. Brooks and T. R. Roberts, Eds., Proceedings of the 9th International Congress on Pesticide Chemistry, The Royal Society of Chemistry, Cambridge, 1999, pp. 120-133. See also U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp 81-96; Hance et al., Weed Control Handbook, 8th Ed., Blackwell Scientific Publications, Oxford, 1989; and Developments in formulation technology, PJB Publications, Richmond, UK, 2000.

In the following Examples, all percentages are by weight and all formulations are prepared in conventional ways. Compound numbers refer to compounds in Index Tables A-B. Without further elaboration, it is believed that one skilled in the art using the preceding description can utilize the present invention to its fullest extent. The following Examples are, therefore, to be constructed as merely illustrative, and not limiting of the disclosure in any way whatsoever. Percentages are by weight except where otherwise indicated.

### Example A

High Strength Concentrate

| | |
|---|---|
| Compound 1 | 98.5% |
| silica aerogel | 0.5% |
| synthetic amorphous fine silica | 1.0% |

### Example B

Wettable Powder

| | |
|---|---|
| Compound 10 | 65.0% |
| dodecylphenol polyethylene glycol ether | 2.0% |
| sodium ligninsulfonate | 4.0% |
| sodium silicoaluminate | 6.0% |
| montmorillonite (calcined) | 23.0% |

### Example C

Granule

| | |
|---|---|
| Compound 42 | 10.0% |
| attapulgite granules (low volatile matter, | 90.0% |
| 0.71/0.30 mm; U.S.S. No. 25-50 sieves) | |

### Example D

Extruded Pellet

| | |
|---|---|
| Compound 9 | 25.0% |
| anhydrous sodium sulfate | 10.0% |
| crude calcium ligninsulfonate | 5.0% |
| sodium alkylnaphthalenesulfonate | 1.0% |
| calcium/magnesium bentonite | 59.0% |

### Example E

Emulsifiable Concentrate

| | |
|---|---|
| Compound 10 | 10.0% |
| polyoxyethylene sorbitol hexoleate | 20.0% |
| C₆-C₁₀ fatty acid methyl ester | 70.0% |

### Example F

Microemulsion

| | |
|---|---|
| Compound 11 | 5.0% |
| polyvinylpyrrolidone-vinyl acetate copolymer | 30.0% |
| alkylpolyglycoside | 30.0% |
| glyceryl monooleate | 15.0% |
| water | 20.0% |

### Example G

Seed Treatment

| | |
|---|---|
| Compound 3 | 20.00% |
| polyvinylpyrrolidone-vinyl acetate copolymer | 5.00% |
| montan acid wax | 5.00% |
| calcium ligninsulfonate | 1.00% |
| polyoxyethylene/polyoxypropylene block copolymers | 1.00% |
| stearyl alcohol (POE 20) | 2.00% |
| polyorganosilane | 0.20% |
| colorant red dye | 0.05% |
| water | 65.75% |

Water-soluble and water-dispersible formulations are typically diluted with water to form aqueous compositions before application. Aqueous compositions for direct applications to the plant or portion thereof (e.g., spray tank compositions) typically at least about 1 ppm or more (e.g., from 1 ppm to 100 ppm) of the compound(s) of this invention.

The compounds of this invention are useful as plant disease control agents. The present invention therefore further comprises a method for controlling plant diseases caused by fungal plant pathogens comprising applying to the plant or portion thereof to be protected, or to the plant seed to be protected, an effective amount of a compound of the invention or a fungicidal composition containing said compound. The compounds and/or compositions of this invention provide control of diseases caused by a broad spectrum of fungal plant pathogens in the Basidiomycete, Ascomycete, Oomycete and Deuteromycete classes. They are effective in controlling a broad spectrum of plant diseases, particularly foliar pathogens of ornamental, turf, vegetable, field, cereal, and fruit crops. These pathogens include: Oomycetes, including Phytophthora diseases such as Phytophthora infestans, Phytophthora megasperma, Phytophthora parasitica, Phytophthora cinnamomi and Phytophthora capsici, Pythium diseases such as Pythium aphanidermatum, and diseases in the Peronosporaceae family such as Plasmopara viticola, Peronospora spp. (including Peronospora tabacina and Peronospora parasitica), Pseudoperonospora spp. (including Pseudoperonospora cubensis) and Bremia lactucae; Ascomycetes, including Alternaria diseases such as Alternaria solani and Alternaria brassicae, Guignardia diseases such as Guignardia bidwell, Venturia diseases such as Venturia inaequalis, Septoria diseases such as Septoria nodorum and Septoria tritici, powdery mildew diseases such as Erysiphe spp. (including Erysiphe graminis and Erysiphe polygoni), Uncinula necatur, Sphaerotheca fuligena and Podosphaera leucotricha, Pseudocercosporella herpotrichoides, Botrytis diseases such as Botrytis cinerea, Monilinia fructicola, Sclerotinia diseases such as Sclerotinia sclerotiorum, Magnaporthe grisea, Phomopsis viticola, Helminthosporium diseases such as Helminthosporium tritici repentis, Pyrenophora teres, anthracnose diseases such as Glomerella or Colletotrichum spp. (such as Colletotrichum graminicola and Colletotrichum orbiculare), and Gaeumannomyces graminis; Basidiomycetes, including rust diseases caused by Puccinia spp. (such as Puccinia recondite, Puccinia striiformis, Puccinia hordei, Puccinia graminis and Puccinia arachidis), Hemileia vastatrix and Phakopsora pachyrhizi; other pathogens including Rutstroemia floccosum (also known as Sclerontina homoeocarpa); Rhizoctonia spp. (such as Rhizoctonia solani); Fusarium diseases such as Fusarium roseum, Fusarium graminearum and Fusarium oxysporum; Verticillium dahliae; Sclerotium rolfsii; Rynchosporium secalis; Cercosporidium personatum, Cercospora arachidicola and Cercospora beticola; and other genera and species closely related to these pathogens. In addition to their fungicidal activity, the compositions or combinations also have activity against bacteria such as Erwinia amylovora, Xanthomonas campestris, Pseudomonas syringae, and other related species.

Plant disease control is ordinarily accomplished by applying an effective amount of a compound of this invention either pre- or post-infection, to the portion of the plant to be protected such as the roots, stems, foliage, fruit, seeds, tubers or bulbs, or to the media (soil or sand) in which the plants to be protected are growing. The compounds can also be applied to seeds to protect the seeds and seedlings developing from the seeds. The compounds can also be applied through irrigation water to treat plants.

Rates of application for these compounds (i.e. a fungicidally effective amount) can be influenced by factors such as the plant diseases to be controlled, the plant species to be protected, ambient moisture and temperature and should be determined under actual use conditions. One skilled in the art can easily determine through simple experimentation the fungicidally effective amount necessary for the desired level of plant disease control. Foliage can normally be protected when treated at a rate of from less than about 1 g/ha to about 5,000 g/ha of active ingredient. Seed and seedlings can normally be protected when seed is treated at a rate of from about 0.1 to about 10 g per kilogram of seed.

Compounds of this invention can also be mixed with one or more other biologically active compounds or agents including fungicides, insecticides, nematocides, bactericides, acaricides, herbicides, herbicide safeners, growth regulators such as insect molting inhibitors and rooting stimulants, chemosterilants, semiochemicals, repellents, attractants, pheromones, feeding stimulants, plant nutrients, other biologically active compounds or entomopathogenic bacteria, virus or fungi to form a multi-component pesticide giving an even broader spectrum of agricultural protection. Thus the present invention also pertains to a composition comprising a compound of Formula 1 (in a fungicidally effective amount) and at least one additional biologically active compound or agent (in a biologically effective amount) and can further comprise at least one of a surfactant, a solid diluent or a liquid diluent. The other biologically active compounds or agents can be formulated in compositions comprising at least one of a surfactant, solid or liquid diluent. For mixtures of the present invention, one or more other biologically active compounds or agents can be formulated together with a compound of Formula 1, to form a premix, or one or more other biologically active compounds or agents can be formulated separately from the compound of Formula 1, and the formulations combined together before application (e.g., in a spray tank) or, alternatively, applied in succession.

Of note is a composition which in addition to the compound of Formula 1 include at least one fungicidal compound selected from the group consisting of the classes (1) methyl benzimidazole carbamate (MBC) fungicides; (2) dicarboximide fungicides; (3) demethylation inhibitor (DMI) fungicides; (4) phenylamide fungicides; (5) amine/morpholine fungicides; (6) phospholipid biosynthesis inhibitor fungicides; (7) carboxamide fungicides; (8) hydroxy(2-amino-)pyrimidine fungicides; (9) anilinopyrimidine fungicides; (10) N-phenyl carbamate fungicides; (11) quinone outside inhibitor (QoI) fungicides; (12) phenylpyrrole fungicides; (13) quinoline fungicides; (14) lipid peroxidation inhibitor fungicides; (15) melanin biosynthesis inhibitors-reductase (MBI-R) fungicides; (16) melanin biosynthesis inhibitors-dehydratase (MBI-D) fungicides; (17) hydroxyanilide fungicides; (18) squalene-epoxidase inhibitor fungicides; (19) polyoxin fungicides; (20) phenylurea fungicides; (21) quinone inside inhibitor (QiI) fungicides; (22) benzamide fungicides; (23) enopyranuronic acid antibiotic fungicides; (24) hexopyranosyl antibiotic fungicides; (25) glucopyranosyl antibiotic: protein synthesis fungicides; (26) glucopyranosyl antibiotic: trehalase and inositol biosynthesis fungicides; (27) cyanoacetamideoxime fungicides; (28) carbamate fungicides; (29) oxidative phosphorylation uncoupling fungicides; (30) organo tin fungicides; (31) carboxylic acid fungicides; (32) heteroaromatic fungicides; (33) phosphonate fungicides; (34) phthalamic acid fungicides; (35) benzotriazine fungicides; (36) benzene-sulfonamide fungicides; (37) pyridazinone fungicides; (38) thiophene-carboxamide fungicides; (39) pyrimidinamide fungicides; (40) carboxylic acid amide (CAA) fungicides; (41) tetracycline antibiotic fungicides; (42) thiocarbamate fungicides; (43) benzamide fungicides; (44) host plant defense induction fungicides; (45) multi-site contact activity fungicides; (46) fungicides other than classes (1) through (45); and salts of compounds of classes (1) through (46).

Further descriptions of these classes of fungicidal compounds are provided below.

(1) “Methyl benzimidazole carbamate (MBC) fungicides” (Fungicide Resistance Action Committee (FRAC) code 1) inhibit mitosis by binding to β-tubulin during microtubule assembly. Inhibition of microtubule assembly can disrupt cell division, transport within the cell and cell structure. Methyl benzimidazole carbamate fungicides include benzimidazole and thiophanate fungicides. The benzimidazoles include benomyl, carbendazim, fuberidazole and thiabendazole. The thiophanates include thiophanate and thiophanate-methyl.

(2) “Dicarboximide fungicides” (Fungicide Resistance Action Committee (FRAC) code 2) are proposed to inhibit a lipid peroxidation in fungi through interference with NADH cytochrome c reductase. Examples include chlozolinate, iprodione, procymidone and vinclozolin.

(3) “Demethylation inhibitor (DMI) fungicides” (Fungicide Resistance Action Committee (FRAC) code 3) inhibit C14-demethylase, which plays a role in sterol production. Sterols, such as ergosterol, are needed for membrane structure and function, making them essential for the development of functional cell walls. Therefore, exposure to these fungicides results in abnormal growth and eventually death of sensitive fungi. DMI fungicides are divided between several chemical classes: azoles (including triazoles and imidazoles), pyrimidines, piperazines and pyridines. The triazoles include azaconazole, bitertanol, bromuconazole, cyproconazole, difenoconazole, diniconazole (including diniconazole-M), epoxiconazole, fenbuconazole, fluquinconazole, flusilazole, flutriafol, hexaconazole, imibenconazole, ipconazole, metconazole, myclobutanil, penconazole, propiconazole, prothioconazole, simeconazole, tebuconazole, tetraconazole, triadimefon, triadimenol, triticonazole and uniconazole. The imidazoles include clotrimazole, imazalil, oxpoconazole, prochloraz, pefurazoate and triflumizole. The pyrimidines include fenarimol and nuarimol. The piperazines include triforine. The pyridines include pyrifenox. Biochemical investigations have shown that all of the above mentioned fungicides are DMI fungicides as described by K. H. Kuck et al., in Modern Selective Fungicides—Properties, Applications and Mechanisms of Action, H. Lyr (Ed.), Gustav Fischer Verlag: New York, 1995, 205-258.

(4) “Phenylamide fungicides” (Fungicide Resistance Action Committee (FRAC) code 4) are specific inhibitors of RNA polymerase in Oomycete fungi. Sensitive fungi exposed to these fungicides show a reduced capacity to incorporate uridine into rRNA. Growth and development in sensitive fungi is prevented by exposure to this class of fungicide. Phenylamide fungicides include acylalanine, oxazolidinone and butyrolactone fungicides. The acylalanines include benalaxyl, benalaxyl-M, furalaxyl, metalaxyl and metalaxyl-M/mefenoxam. The oxazolidinones include oxadixyl. The butyrolactones include ofurace.

(5) “Amine/morpholine fungicides” (Fungicide Resistance Action Committee (FRAC) code 5) inhibit two target sites within the sterol biosynthetic pathway, Δ⁸→Δ⁷ isomerase and Δ¹⁴ reductase. Sterols, such as ergosterol, are needed for membrane structure and function, making them essential for the development of functional cell walls. Therefore, exposure to these fungicides results in abnormal growth and eventually death of sensitive fungi. Amine/morpholine fungicides (also known as non-DMI sterol biosynthesis inhibitors) include morpholine, piperidine and spiroketal-amine fungicides. The morpholines include aldimorph, dodemorph, fenpropimorph, tridemorph and trimorphamide. The piperidines include fenpropidin and piperalin. The spiroketal-amines include spiroxamine.

(6) “Phospholipid biosynthesis inhibitor fungicides” (Fungicide Resistance Action Committee (FRAC) code 6) inhibit growth of fungi by affecting phospholipid biosynthesis. Phospholipid biosynthesis fungicides include phosphorothiolate and dithiolane fungicides. The phosphorothiolates include edifenphos, iprobenfos and pyrazophos. The dithiolanes include isoprothiolane.

(7) “Carboxamide fungicides” (Fungicide Resistance Action Committee (FRAC) code 7) inhibit Complex II (succinate dehydrogenase) fungal respiration by disrupting a key enzyme in the Krebs Cycle (TCA cycle) named succinate dehydrogenase. Inhibiting respiration prevents the fungus from making ATP, and thus inhibits growth and reproduction. Carboxamide fungicides include benzamides, furan carboxamides, oxathiin carboxamides, thiazole carboxamides, pyrazole carboxamides and pyridine carboxamides. The benzamides include benodanil, flutolanil and mepronil. The furan carboxamides include fenfuram. The oxathiin carboxamides include carboxin and oxycarboxin. The thiazole carboxamides include thifluzamide. The pyrazole carboxamides include furametpyr, penthiopyrad, bixafen, isopyrazam, sedaxane and penflufen. The pyridine carboxamides include boscalid.

(8) “Hydroxy(2-amino-)pyrimidine fungicides” (Fungicide Resistance Action Committee (FRAC) code 8) inhibit nucleic acid synthesis by interfering with adenosine deaminase. Examples include bupirimate, dimethirimol and ethirimol.

(9) “Anilinopyrimidine fungicides” (Fungicide Resistance Action Committee (FRAC) code 9) are proposed to inhibit biosynthesis of the amino acid methionine and to disrupt the secretion of hydrolytic enzymes that lyse plant cells during infection. Examples include cyprodinil, mepanipyrim and pyrimethanil.

(10) “N-Phenyl carbamate fungicides” (Fungicide Resistance Action Committee (FRAC) code 10) inhibit mitosis by binding to β-tubulin and disrupting microtubule assembly. Inhibition of microtubule assembly can disrupt cell division, transport within the cell and cell structure. Examples include diethofencarb.

(11) “Quinone outside inhibitor (QoI) fungicides” (Fungicide Resistance Action Committee (FRAC) code 11) inhibit Complex III mitochondrial respiration in fungi by affecting ubiquinol oxidase. Oxidation of ubiquinol is blocked at the “quinone outside” (Q_{O}) site of the cytochrome bc₁ complex, which is located in the inner mitochondrial membrane of fungi. Inhibiting mitochondrial respiration prevents normal fungal growth and development. Quinone outside inhibitor fungicides (also known as strobilurin fungicides) include methoxyacrylate, methoxycarbamate, oximinoacetate, oximinoacetamide, oxazolidinedione, dihydrodioxazine, imidazolinone and benzylcarbamate fungicides. The methoxyacrylates include azoxystrobin, enestroburin (SYP-Z071), picoxystrobin and pyraoxystrobin (SYP-3343). The methoxycarbamates include pyraclostrobin and pyrametostrobin (SYP-4155). The oximinoacetates include kresoxim-methyl and trifloxystrobin. The oximinoacetamides include dimoxystrobin, metominostrobin, orysastrobin, α-[methoxyimino]-N-methyl-2-[[[1-[3-(trifluoromethyl)phenyl]ethoxy]imino]-methyl]benzeneacetamide and 2-[[[3-(2,6-dichlorophenyl)-1-methyl-2-propen-1-ylidene]-amino]oxy]methyl]-α-(methoxyimino)-N-methylbenzeneacetamide. The oxazolidinediones include famoxadone. The dihydrodioxazines include fluoxastrobin. The imidazolinones include fenamidone. The benzylcarbamates include pyribencarb.

(12) “Phenylpyrrole fungicides” (Fungicide Resistance Action Committee (FRAC) code 12) inhibit a MAP protein kinase associated with osmotic signal transduction in fungi. Fenpiclonil and fludioxonil are examples of this fungicide class.

(13) “Quinoline fungicides” (Fungicide Resistance Action Committee (FRAC) code 13) are proposed to inhibit signal transduction by affecting G-proteins in early cell signaling. They have been shown to interfere with germination and/or appressorium formation in fungi that cause powder mildew diseases. Quinoxyfen and tebufloquin are examples of this class of fungicide.

(14) “Lipid peroxidation inhibitor fungicides” (Fungicide Resistance Action Committee (FRAC) code 14) are proposed to inhibit lipid peroxidation which affects membrane synthesis in fungi. Members of this class, such as etridiazole, may also affect other biological processes such as respiration and melanin biosynthesis. Lipid peroxidation fungicides include aromatic carbon and 1,2,4-thiadiazole fungicides. The aromatic carbon fungicides include biphenyl, chloroneb, dicloran, quintozene, tecnazene and tolclofos-methyl. The 1,2,4-thiadiazole fungicides include etridiazole.

(15) “Melanin biosynthesis inhibitors-reductase (MBI-R) fungicides” (Fungicide Resistance Action Committee (FRAC) code 16.1) inhibit the naphthal reduction step in melanin biosynthesis. Melanin is required for host plant infection by some fungi. Melanin biosynthesis inhibitors-reductase fungicides include isobenzofuranone, pyrroloquinolinone and triazolobenzothiazole fungicides. The isobenzofuranones include fthalide. The pyrroloquinolinones include pyroquilon. The triazolobenzothiazoles include tricyclazole.

(16) “Melanin biosynthesis inhibitors-dehydratase (MBI-D) fungicides” (Fungicide Resistance Action Committee (FRAC) code 16.2) inhibit scytalone dehydratase in melanin biosynthesis. Melanin in required for host plant infection by some fungi. Melanin biosynthesis inhibitors-dehydratase fungicides include cyclopropanecarboxamide, carboxamide and propionamide fungicides. The cyclopropanecarboxamides include carpropamid. The carboxamides include diclocymet. The propionamides include fenoxanil.

(17) “Hydroxyanilide fungicides (Fungicide Resistance Action Committee (FRAC) code 17) inhibit C4-demethylase which plays a role in sterol production. Examples include fenhexamid.

(18) “Squalene-epoxidase inhibitor fungicides” (Fungicide Resistance Action Committee (FRAC) code 18) inhibit squalene-epoxidase in ergosterol biosynthesis pathway. Sterols such as ergosterol are needed for membrane structure and function, making them essential for the development of functional cell walls. Therefore exposure to these fungicides results in abnormal growth and eventually death of sensitive fungi. Squalene-epoxidase inhibitor fungicides include thiocarbamate and allylamine fungicides. The thiocarbamates include pyributicarb. The allylamines include naftifine and terbinafine.

(19) “Polyoxin fungicides” (Fungicide Resistance Action Committee (FRAC) code 19) inhibit chitin synthase. Examples include polyoxin.

(20) “Phenylurea fungicides” (Fungicide Resistance Action Committee (FRAC) code 20) are proposed to affect cell division. Examples include pencycuron.

(21) “Quinone inside inhibitor (QiI) fungicides” (Fungicide Resistance Action Committee (FRAC) code 21) inhibit Complex III mitochondrial respiration in fungi by affecting ubiquinol reductase. Reduction of ubiquinol is blocked at the “quinone inside” (Qᵢ) site of the cytochrome bc₁ complex, which is located in the inner mitochondrial membrane of fungi. Inhibiting mitochondrial respiration prevents normal fungal growth and development. Quinone inside inhibitor fungicides include cyanoimidazole and sulfamoyltriazole fungicides. The cyanoimidazoles include cyazofamid. The sulfamoyltriazoles include amisulbrom.

(22) “Benzamide fungicides” (Fungicide Resistance Action Committee (FRAC) code 22) inhibit mitosis by binding to β-tubulin and disrupting microtubule assembly Inhibition of microtubule assembly can disrupt cell division, transport within the cell and cell structure. Examples include zoxamide.

(23) “Enopyranuronic acid antibiotic fungicides” (Fungicide Resistance Action Committee (FRAC) code 23) inhibit growth of fungi by affecting protein biosynthesis. Examples include blasticidin-S.

(24) “Hexopyranosyl antibiotic fungicides” (Fungicide Resistance Action Committee (FRAC) code 24) inhibit growth of fungi by affecting protein biosynthesis. Examples include kasugamycin.

(25) “Glucopyranosyl antibiotic: protein synthesis fungicides” (Fungicide Resistance Action Committee (FRAC) code 25) inhibit growth of fungi by affecting protein biosynthesis. Examples include streptomycin.

(26) “Glucopyranosyl antibiotic: trehalase and inositol biosynthesis fungicides” (Fungicide Resistance Action Committee (FRAC) code 26) inhibit trehalase in inositol biosynthesis pathway. Examples include validamycin.

(27) “Cyanoacetamideoxime fungicides (Fungicide Resistance Action Committee (FRAC) code 27) include cymoxanil.

(28) “Carbamate fungicides” (Fungicide Resistance Action Committee (FRAC) code 28) are considered multi-site inhibitors of fungal growth. They are proposed to interfere with the synthesis of fatty acids in cell membranes, which then disrupts cell membrane permeability. Propamacarb, propamacarb-hydrochloride, iodocarb, and prothiocarb are examples of this fungicide class.

(29) “Oxidative phosphorylation uncoupling fungicides” (Fungicide Resistance Action Committee (FRAC) code 29) inhibit fungal respiration by uncoupling oxidative phosphorylation. Inhibiting respiration prevents normal fungal growth and development. This class includes 2,6-dinitroanilines such as fluazinam, pyrimidonehydrazones such as ferimzone and dinitrophenyl crotonates such as dinocap, meptyldinocap and binapacryl.

(30) “Organo tin fungicides” (Fungicide Resistance Action Committee (FRAC) code 30) inhibit adenosine triphosphate (ATP) synthase in oxidative phosphorylation pathway. Examples include fentin acetate, fentin chloride and fentin hydroxide.

(31) “Carboxylic acid fungicides” (Fungicide Resistance Action Committee (FRAC) code 31) inhibit growth of fungi by affecting deoxyribonucleic acid (DNA) topoisomerase type II (gyrase). Examples include oxolinic acid.

(32) “Heteroaromatic fungicides” (Fungicide Resistance Action Committee (FRAC) code 32) are proposed to affect DNA/ribonucleic acid (RNA) synthesis. Heteroaromatic fungicides include isoxazole and isothiazolone fungicides. The isoxazoles include hymexazole and the isothiazolones include octhilinone.

(33) “Phosphonate fungicides” (Fungicide Resistance Action Committee (FRAC) code 33) include phosphorous acid and its various salts, including fosetyl-aluminum.

(34) “Phthalamic acid fungicides” (Fungicide Resistance Action Committee (FRAC) code 34) include teclofthalam.

(35) “Benzotriazine fungicides” (Fungicide Resistance Action Committee (FRAC) code 35) include triazoxide.

(36) “Benzene-sulfonamide fungicides” (Fungicide Resistance Action Committee (FRAC) code 36) include flusulfamide.

(37) “Pyridazinone fungicides” (Fungicide Resistance Action Committee (FRAC) code 37) include diclomezine.

(38) “Thiophene-carboxamide fungicides” (Fungicide Resistance Action Committee (FRAC) code 38) are proposed to affect ATP production. Examples include silthiofam.

(39) “Pyrimidinamide fungicides” (Fungicide Resistance Action Committee (FRAC) code 39) inhibit growth of fungi by affecting phospholipid biosynthesis and include diflumetorim.

(40) “Carboxylic acid amide (CAA) fungicides” (Fungicide Resistance Action Committee (FRAC) code 40) are proposed to inhibit phospholipid biosynthesis and cell wall deposition. Inhibition of these processes prevents growth and leads to death of the target fungus. Carboxylic acid amide fungicides include cinnamic acid amide, valinamide carbamate and mandelic acid amide fungicides. The cinnamic acid amides include dimethomorph and flumorph. The valinamide carbamates include benthiavalicarb, benthiavalicarb-isopropyl, iprovalicarb, valifenalate and valiphenal. The mandelic acid amides include mandipropamid, N-[2-[4-[[3-(4-chlorophenyl)-2-propyn-1-yl]oxy]-3-methoxyphenyl]ethyl]-3-methyl-2-[(methylsulfonyl)amino]butanamide and N-[2-[4-[[3-(4-chlorophenyl)-2-propyn-1-yl]oxy]-3-methoxyphenyl]ethyl]-3-methyl-2-[(ethylsulfonyl)amino]butanamide.

(41) “Tetracycline antibiotic fungicides” (Fungicide Resistance Action Committee (FRAC) code 41) inhibit growth of fungi by affecting complex 1 nicotinamide adenine dinucleotide (NADH) oxidoreductase. Examples include oxytetracycline.

(42) “Thiocarbamate fungicides” (Fungicide Resistance Action Committee (FRAC) code 42) include methasulfocarb.

(43) “Benzamide fungicides” (Fungicide Resistance Action Committee (FRAC) code 43) inhibit growth of fungi by delocalization of spectrin-like proteins. Examples include acylpicolide fungicides such as fluopicolide and fluopyram.

(44) “Host plant defense induction fungicides” (Fungicide Resistance Action Committee (FRAC) code P) induce host plant defense mechanisms. Host plant defense induction fungicides include benzo-thiadiazole, benzisothiazole and thiadiazole-carboxamide fungicides. The benzo-thiadiazoles include acibenzolar-5-methyl. The benzisothiazoles include probenazole. The thiadiazole-carboxamides include tiadinil and isotianil.

(45) “Multi-site contact fungicides” inhibit fungal growth through multiple sites of action and have contact/preventive activity. This class of fungicides includes: (45.1) “copper fungicides” (Fungicide Resistance Action Committee (FRAC) code M1)”, (45.2) “sulfur fungicides” (Fungicide Resistance Action Committee (FRAC) code M2), (45.3) “dithiocarbamate fungicides” (Fungicide Resistance Action Committee (FRAC) code M3), (45.4) “phthalimide fungicides” (Fungicide Resistance Action Committee (FRAC) code M4), (45.5) “chloronitrile fungicides” (Fungicide Resistance Action Committee (FRAC) code M5), (45.6) “sulfamide fungicides” (Fungicide Resistance Action Committee (FRAC) code M6), (45.7) “guanidine fungicides” (Fungicide Resistance Action Committee (FRAC) code M7), (45.8) “triazine fungicides” (Fungicide Resistance Action Committee (FRAC) code M8) and (45.9) “quinone fungicides” (Fungicide Resistance Action Committee (FRAC) code M9). “Copper fungicides” are inorganic compounds containing copper, typically in the copper(II) oxidation state; examples include copper oxychloride, copper sulfate and copper hydroxide, including compositions such as Bordeaux mixture (tribasic copper sulfate). “Sulfur fungicides” are inorganic chemicals containing rings or chains of sulfur atoms; examples include elemental sulfur. “Dithiocarbamate fungicides” contain a dithiocarbamate molecular moiety; examples include mancozeb, metiram, propineb, ferbam, maneb, thiram, zineb and ziram. “Phthalimide fungicides” contain a phthalimide molecular moiety; examples include folpet, captan and captafol. “Chloronitrile fungicides” contain an aromatic ring substituted with chloro and cyano; examples include chlorothalonil. “Sulfamide fungicides” include dichlofluanid and tolyfluanid. “Guanidine fungicides” include dodine, guazatine, iminoctadine albesilate and iminoctadine triacetate. “Triazine fungicides” include anilazine. “Quinone fungicides” include dithianon.

(46) “Fungicides other than fungicides of classes (1) through (45)” include certain fungicides whose mode of action may be unknown. These include: (46.1) “thiazole carboxamide fungicides” (Fungicide Resistance Action Committee (FRAC) code U5), (46.2) “phenyl-acetamide fungicides” (Fungicide Resistance Action Committee (FRAC) code U6), (46.3) “quinazolinone fungicides” (Fungicide Resistance Action Committee (FRAC) code U7), (46.4) “benzophenone fungicides” (Fungicide Resistance Action Committee (FRAC) code U8) and (46.5) “triazolopyrimidine fungicides”. The thiazole carboxamides include ethaboxam. The phenyl-acetamides include cyflufenamid and N-[[(cyclopropylmethoxy)-amino][6-(difluoromethoxy)-2,3-difluorophenyl]-methylene]benzeneacetamide. The quinazolinones include proquinazid. The benzophenones include metrafenone. The triazolopyrimidines include ametoctradin. Class (46) (i.e. “Fungicides other than classes (1) through (45)”) also includes bethoxazin, fluxapyroxad, neo-asozin (ferric methanearsonate), pyriofenone, pyrrolnitrin, quinomethionate, tebufloquin, N-[2-[4-[[3-(4-chlorophenyl)-2-propyn-1-yl]oxy]-3-methoxyphenyl]ethyl]-3-methyl-2-[(methylsulfonyl)amino]butanamide, N-[2-[4-[[3-(4-chlorophenyl)-2-propyn-1-yl]oxy]-3-methoxyphenyl]ethyl]-3-methyl-2-[(ethylsulfonyl)amino]butanamide, 2-[[2-fluoro-5-(trifluoromethyl)phenyl]thio]-2-[3-(2-methoxyphenyl)-2-thiazolidinylidene]acetonitrile, 3-[5-(4-chlorophenyl)-2,3-dimethyl-3-isoxazolidinyl]pyridine, 4-fluorophenyl N-[1-[[[1-(4-cyanophenyl)ethyl]sulfonyl]methyl]-propyl]carbamate, 5-chloro-6-(2,4,6-trifluorophenyl)-7-(4-methylpiperidin-1-yl)[1,2,4]-triazolo[1,5-α]pyrimidine, N-(4-chloro-2-nitrophenyl)-N-ethyl-4-methylbenzenesulfonamide, N-[[(cyclopropylmethoxy)amino][6-(difluoromethoxy)-2,3-difluorophenyl]methylene]-benzeneacetamide, N-[4-[4-chloro-3-(trifluoromethyl)phenoxy]-2,5-dimethylphenyl]-N-ethyl-N-methylmethanimidamide, 1-[(2-propenylthio)carbonyl]-2-(1-methylethyl)-4-(2-methylphenyl)-5-amino-1H-pyrazol-3-one, N-[9-(dichloromethylene)-1,2,3,4-tetrahydro-1,4-methanonaphthalen-5-yl]-3-(difluoromethyl)-1-methyl-1H-pyrazole-4-carboxamide, 3-(di-fluoromethyl)-N-[9-(difluoromethylene)-1,2,3,4-tetrahydro-1,4-methanonaphthalen-5-yl]-1-methyl-1H-pyrazole-4-carboxamide, N-[9-(dichloromethylene)-1,2,3,4-tetrahydro-1,4-methanonaphthalen-5-yl]-1-methyl-3-(trifluoromethyl)-1H-pyrazole-4-carboxamide and N-[4-[[3-[(4-chlorophenyl)methyl]-1,2,4-thiadiazol-5-yl]oxy]-2,5-dimethylphenyl]-N-ethyl-N-methylmethanimidamide.

Therefore of note is a mixture (i.e. composition) comprising a compound of Formula 1 and at least one fungicidal compound selected from the group consisting of the aforedescribed classes (1) through (46). Also of note is a composition comprising said mixture (in fungicidally effective amount) and further comprising at least one additional component selected from the group consisting of surfactants, solid diluents and liquid diluents. Of particular note is a mixture (i.e. composition) comprising a compound of Formula 1 and at least one fungicidal compound selected from the group of specific compounds listed above in connection with classes (1) through (46). Also of particular note is a composition comprising said mixture (in fungicidally effective amount) and further comprising at least one additional surfactant selected from the group consisting of surfactants, solid diluents and liquid diluents.

Examples of other biologically active compounds or agents with which compounds of this invention can be formulated are: insecticides such as abamectin, acephate, acetamiprid, acrinathrin, amidoflumet (S-1955), avermectin, azadirachtin, azinphos-methyl, bifenthrin, bifenazate, buprofezin, carbofuran, cartap, chlorantraniliprole, chlorfenapyr, chlorfluazuron, chlorpyrifos, chlorpyrifos-methyl, chromafenozide, clothianidin, cyantraniliprole (3-bromo-1-(3-chloro-2-pyridinyl)-N-[4-cyano-2-methyl-6-[(methylamino)carbonyl]phenyl]-1H-pyrazole-5-carboxamide), cyflumetofen, cyfluthrin, beta-cyfluthrin, cyhalothrin, lambda-cyhalothrin, cypermethrin, cyromazine, deltamethrin, diafenthiuron, diazinon, dieldrin, diflubenzuron, dimefluthrin, dimethoate, dinotefuran, diofenolan, emamectin, endosulfan, esfenvalerate, ethiprole, fenothiocarb, fenoxycarb, fenpropathrin, fenvalerate, fipronil, flonicamid, flubendiamide, flucythrinate, tau-fluvalinate, flufenerim (UR-50701), flufenoxuron, fonophos, halofenozide, hexaflumuron, hydramethylnon, imidacloprid, indoxacarb, isofenphos, lufenuron, malathion, meperfluthrin, metaflumizone, metaldehyde, methamidophos, methidathion, methomyl, methoprene, methoxychlor, methoxyfenozide, metofluthrin, milbemycin oxime, monocrotophos, nicotine, nitenpyram, nithiazine, novaluron, noviflumuron (XDE-007), oxamyl, parathion, parathion-methyl, permethrin, phorate, phosalone, phosmet, phosphamidon, pirimicarb, profenofos, profluthrin, pymetrozine, pyrafluprole, pyrethrin, pyridalyl, pyrifluquinazon, pyriprole, pyriproxyfen, rotenone, ryanodine, spinetoram, spinosad, spirodiclofen, spiromesifen (BSN 2060), spirotetramat, sulfoxaflor, sulprofos, tebufenozide, teflubenzuron, tefluthrin, terbufos, tetrachlorvinphos, tetramethylfluthrin, thiacloprid, thiamethoxam, thiodicarb, thiosultap-sodium, tolfenpyrad, tralomethrin, triazamate, trichlorfon and triflumuron; and biological agents including entomopathogenic bacteria, such as Bacillus thuringiensis subsp. aizawai, Bacillus thuringiensis subsp. kurstaki, and the encapsulated delta-endotoxins of Bacillus thuringiensis (e.g., Cellcap, MPV, MPVII); entomopathogenic fungi, such as green muscardine fungus; and entomopathogenic virus including baculovirus, nucleopolyhedro virus (NPV) such as HzNPV, AfNPV; and granulosis virus (GV) such as CpGV.

Compounds of this invention and compositions thereof can be applied to plants genetically transformed to express proteins toxic to invertebrate pests (such as Bacillus thuringiensis delta-endotoxins). The effect of the exogenously applied fungicidal compounds of this invention may be synergistic with the expressed toxin proteins.

General references for agricultural protectants (i.e. insecticides, fungicides, nematocides, acaricides, herbicides and biological agents) include The Pesticide Manual, 13th Edition, C. D. S. Tomlin, Ed., British Crop Protection Council, Farnham, Surrey, U.K., 2003 and The BioPesticide Manual, 2nd Edition, L. G. Copping, Ed., British Crop Protection Council, Farnham, Surrey, U.K., 2001.

For embodiments where one or more of these various mixing partners are used, the weight ratio of these various mixing partners (in total) to the compound of Formula 1 is typically between about 1:3000 and about 3000:1. Of note are weight ratios between about 1:300 and about 300:1 (for example ratios between about 1:30 and about 30:1). One skilled in the art can easily determine through simple experimentation the biologically effective amounts of active ingredients necessary for the desired spectrum of biological activity. It will be evident that including these additional components may expand the spectrum of diseases controlled beyond the spectrum controlled by the compound of Formula 1 alone.

In certain instances, combinations of a compound of this invention with other biologically active (particularly fungicidal) compounds or agents (i.e. active ingredients) can result in a greater-than-additive (i.e. synergistic) effect. Reducing the quantity of active ingredients released in the environment while ensuring effective pest control is always desirable. When synergism of fungicidal active ingredients occurs at application rates giving agronomically satisfactory levels of fungal control, such combinations can be advantageous for reducing crop production cost and decreasing environmental load.

Of note is a combination of a compound of Formula 1 with at least one other fungicidal active ingredient. Of particular note is such a combination where the other fungicidal active ingredient has different site of action from the compound of Formula 1. In certain instances, a combination with at least one other fungicidal active ingredient having a similar spectrum of control but a different site of action will be particularly advantageous for resistance management. Thus, a composition of the present invention can further comprise a biologically effective amount of at least one additional fungicidal active ingredient having a similar spectrum of control but a different site of action.

Of particular note are compositions which in addition to compound of Formula 1 include at least one compound selected from the group consisting of (1) alkylenebis(dithiocarbamate) fungicides; (2) cymoxanil; (3) phenylamide fungicides; (4) proquinazid (6-iodo-3-propyl-2-propyloxy-4(3H)-quinazolinone); (5) chlorothalonil; (6) carboxamides acting at complex II of the fungal mitochondrial respiratory electron transfer site; (7) quinoxyfen; (8) metrafenone; (9) cyflufenamid; (10) cyprodinil; (11) copper compounds; (12) phthalimide fungicides; (13) fosetyl-aluminum; (14) benzimidazole fungicides; (15) cyazofamid; (16) fluazinam; (17) iprovalicarb; (18) propamocarb; (19) validomycin; (20) dichlorophenyl dicarboximide fungicides; (21) zoxamide; (22) fluopicolide; (23) mandipropamid; (24) carboxylic acid amides acting on phospholipid biosynthesis and cell wall deposition; (25) dimethomorph; (26) non-DMI sterol biosynthesis inhibitors; (27) inhibitors of demethylase in sterol biosynthesis; (28) bc₁ complex fungicides; and salts of compounds of (1) through (28).

Further descriptions of classes of fungicidal compounds are provided below.

Sterol biosynthesis inhibitors (group (27)) control fungi by inhibiting enzymes in the sterol biosynthesis pathway. Demethylase-inhibiting fungicides have a common site of action within the fungal sterol biosynthesis pathway, involving inhibition of demethylation at position 14 of lanosterol or 24-methylene dihydrolanosterol, which are precursors to sterols in fungi. Compounds acting at this site are often referred to as demethylase inhibitors, DMI fungicides, or DMIs. The demethylase enzyme is sometimes referred to by other names in the biochemical literature, including cytochrome P-450 (14DM). The demethylase enzyme is described in, for example, J. Biol. Chem. 1992, 267, 13175-79 and references cited therein. DMI fungicides are divided between several chemical classes: azoles (including triazoles and imidazoles), pyrimidines, piperazines and pyridines. The triazoles include azaconazole, bromuconazole, cyproconazole, difenoconazole, diniconazole (including diniconazole-M), epoxiconazole, etaconazole, fenbuconazole, fluquinconazole, flusilazole, flutriafol, hexaconazole, imibenconazole, ipconazole, metconazole, myclobutanil, penconazole, propiconazole, prothioconazole, quinconazole, simeconazole, tebuconazole, tetraconazole, triadimefon, triadimenol, triticonazole and uniconazole. The imidazoles include clotrimazole, econazole, imazalil, isoconazole, miconazole, oxpoconazole, prochloraz and triflumizole. The pyrimidines include fenarimol, nuarimol and triarimol. The piperazines include triforine. The pyridines include buthiobate and pyrifenox. Biochemical investigations have shown that all of the above mentioned fungicides are DMI fungicides as described by K. H. Kuck et al. in Modern Selective Fungicides—Properties, Applications and Mechanisms of Action, H. Lyr (Ed.), Gustav Fischer Verlag: New York, 1995, 205-258.

bc₁ Complex Fungicides (group 28) have a fungicidal mode of action which inhibits the bc₁ complex in the mitochondrial respiration chain. The bc₁ complex is sometimes referred to by other names in the biochemical literature, including complex III of the electron transfer chain, and ubihydroquinone:cytochrome c oxidoreductase. This complex is uniquely identified by Enzyme Commission number EC1.10.2.2. The bc₁ complex is described in, for example, J. Biol. Chem. 1989, 264, 14543-48; Methods Enzymol. 1986, 126, 253-71; and references cited therein. Strobilurin fungicides such as azoxystrobin, dimoxystrobin, enestroburin (SYP-Z071), fluoxastrobin, kresoxim-methyl, metominostrobin, orysastrobin, picoxystrobin, pyraclostrobin, pyrametostrobin, pyraoxystrobin and trifloxystrobin are known to have this mode of action (H. Sauter et al., Angew. Chem. Int. Ed. 1999, 38, 1328-1349). Other fungicidal compounds that inhibit the bc₁ complex in the mitochondrial respiration chain include famoxadone and fenamidone.

Alkylenebis(dithiocarbamate)s (group (1)) include compounds such as mancozeb, maneb, propineb and zineb. Phenylamides (group (3)) include compounds such as metalaxyl, benalaxyl, furalaxyl and oxadixyl. Carboxamides (group (6)) include compounds such as boscalid, carboxin, fenfuram, flutolanil, furametpyr, mepronil, oxycarboxin, thifluzamide, penthiopyrad and N-[2-(1,3-dimethylbutyl)phenyl]-5-fluoro-1,3-dimethyl-1H-pyrazole-4-carboxamide (PCT Patent Publication WO 2003/010149), and are known to inhibit mitochondrial function by disrupting complex II (succinate dehydrogenase) in the respiratory electron transport chain. Copper compounds (group (11)) include compounds such as copper oxychloride, copper sulfate and copper hydroxide, including compositions such as Bordeaux mixture (tribasic copper sulfate). Phthalimides (group (12)) include compounds such as folpet and captan. Benzimidazole fungicides (group (14)) include benomyl and carbendazim. Dichlorophenyl dicarboximide fungicides (group (20)) include chlozolinate, dichlozoline, iprodione, isovaledione, myclozolin, procymidone and vinclozolin.

Non-DMI sterol biosynthesis inhibitors (group (26)) include morpholine and piperidine fungicides. The morpholines and piperidines are sterol biosynthesis inhibitors that have been shown to inhibit steps in the sterol biosynthesis pathway at a point later than the inhibitions achieved by the DMI sterol biosynthesis (group (27)). The morpholines include aldimorph, dodemorph, fenpropimorph, tridemorph and trimorphamide. The piperidines include fenpropidin.

Of further note are combinations of compounds of Formula 1 with azoxystrobin, kresoxim-methyl, trifloxystrobin, pyraclostrobin, picoxystrobin, dimoxystrobin, metominostrobin/fenominostrobin, carbendazim, chlorothalonil, quinoxyfen, metrafenone, cyflufenamid, fenpropidine, fenpropimorph, bromuconazole, cyproconazole, difenoconazole, epoxiconazole, fenbuconazole, flusilazole, hexaconazole, ipconazole, metconazole, penconazole, propiconazole, proquinazid, prothioconazole, tebuconazole, triticonazole, famoxadone, prochloraz, penthiopyrad and boscalid (nicobifen).

Specifically preferred mixtures (compound numbers refer to compounds in Index Tables A-B) are selected from the group: combinations of Compound 3, Compound 8, Compound 9, Compound 10, Compound 11, Compound 13, Compound 31, Compound 35, Compound 40, Compound 41, Compound 42, Compound 121, Compound 143, Compound 205, Compound 206, Compound 212, Compound 213, Compound 218, Compound 220, Compound 221, Compound 224, Compound 248, Compound 249, Compound 250, Compound 287, Compound 288, Compound 332 or Compound 350 with azoxystrobin, combinations of Compound 3, Compound 8, Compound 9, Compound 10, Compound 11, Compound 13, Compound 31, Compound 35, Compound 40, Compound 41, Compound 42, Compound 121, Compound 143, Compound 205, Compound 206, Compound 212, Compound 213, Compound 218, Compound 220, Compound 221, Compound 224, Compound 248, Compound 249, Compound 250, Compound 287, Compound 288, Compound 332 or Compound 350 with kresoxim-methyl, combinations of Compound 3, Compound 8, Compound 9, Compound 10, Compound 11, Compound 13, Compound 31, Compound 35, Compound 40, Compound 41, Compound 42, Compound 121, Compound 143, Compound 205, Compound 206, Compound 212, Compound 213, Compound 218, Compound 220, Compound 221, Compound 224, Compound 248, Compound 249, Compound 250, Compound 287, Compound 288, Compound 332 or Compound 350 with trifloxystrobin, combinations of Compound 3, Compound 8, Compound 9, Compound 10, Compound 11, Compound 13, Compound 31, Compound 35, Compound 40, Compound 41, Compound 42, Compound 121, Compound 143, Compound 205, Compound 206, Compound 212, Compound 213, Compound 218, Compound 220, Compound 221, Compound 224, Compound 248, Compound 249, Compound 250, Compound 287, Compound 288, Compound 332 or Compound 350 with picoxystrobin, combinations of Compound 3, Compound 8, Compound 9, Compound 10, Compound 11, Compound 13, Compound 31, Compound 35, Compound 40, Compound 41, Compound 42, Compound 121, Compound 143, Compound 205, Compound 206, Compound 212, Compound 213, Compound 218, Compound 220, Compound 221, Compound 224, Compound 248, Compound 249, Compound 250, Compound 287, Compound 288, Compound 332 or Compound 350 with quinoxyfen, combinations of Compound 3, Compound 8, Compound 9, Compound 10, Compound 11, Compound 13, Compound 31, Compound 35, Compound 40, Compound 41, Compound 42, Compound 121, Compound 143, Compound 205, Compound 206, Compound 212, Compound 213, Compound 218, Compound 220, Compound 221, Compound 224, Compound 248, Compound 249, Compound 250, Compound 287, Compound 288, Compound 332 or Compound 350 with metrafenone, combinations of Compound 3, Compound 8, Compound 9, Compound 10, Compound 11, Compound 13, Compound 31, Compound 35, Compound 40, Compound 41, Compound 42, Compound 121, Compound 143, Compound 205, Compound 206, Compound 212, Compound 213, Compound 218, Compound 220, Compound 221, Compound 224, Compound 248, Compound 249, Compound 250, Compound 287, Compound 288, Compound 332 or Compound 350 with fenpropidine, combinations of Compound 3, Compound 8, Compound 9, Compound 10, Compound 11, Compound 13, Compound 31, Compound 35, Compound 40, Compound 41, Compound 42, Compound 121, Compound 143, Compound 205, Compound 206, Compound 212, Compound 213, Compound 218, Compound 220, Compound 221, Compound 224, Compound 248, Compound 249, Compound 250, Compound 287, Compound 288, Compound 332 or Compound 350 with fenpropimorph, combinations of Compound 3, Compound 8, Compound 9, Compound 10, Compound 11, Compound 13, Compound 31, Compound 35, Compound 40, Compound 41, Compound 42, Compound 121, Compound 143, Compound 205, Compound 206, Compound 212, Compound 213, Compound 218, Compound 220, Compound 221, Compound 224, Compound 248, Compound 249, Compound 250, Compound 287, Compound 288, Compound 332 or Compound 350 with cyproconazole, combinations of Compound 3, Compound 8, Compound 9, Compound 10, Compound 11, Compound 13, Compound 31, Compound 35, Compound 40, Compound 41, Compound 42, Compound 121, Compound 143, Compound 205, Compound 206, Compound 212, Compound 213, Compound 218, Compound 220, Compound 221, Compound 224, Compound 248, Compound 249, Compound 250, Compound 287, Compound 288, Compound 332 or Compound 350 with epoxiconazole, combinations of Compound 3, Compound 8, Compound 9, Compound 10, Compound 11, Compound 13, Compound 31, Compound 35, Compound 40, Compound 41, Compound 42, Compound 121, Compound 143, Compound 205, Compound 206, Compound 212, Compound 213, Compound 218, Compound 220, Compound 221, Compound 224, Compound 248, Compound 249, Compound 250, Compound 287, Compound 288, Compound 332 or Compound 350 with flusilazole, combinations of Compound 3, Compound 8, Compound 9, Compound 10, Compound 11, Compound 13, Compound 31, Compound 35, Compound 40, Compound 41, Compound 42, Compound 121, Compound 143, Compound 205, Compound 206, Compound 212, Compound 213, Compound 218, Compound 220, Compound 221, Compound 224, Compound 248, Compound 249, Compound 250, Compound 287, Compound 288, Compound 332 or Compound 350 with metconazole, combinations of Compound 3, Compound 8, Compound 9, Compound 10, Compound 11, Compound 13, Compound 31, Compound 35, Compound 40, Compound 41, Compound 42, Compound 121, Compound 143, Compound 205, Compound 206, Compound 212, Compound 213, Compound 218, Compound 220, Compound 221, Compound 224, Compound 248, Compound 249, Compound 250, Compound 287, Compound 288, Compound 332 or Compound 350 with propiconazole, combinations of Compound 3, Compound 8, Compound 9, Compound 10, Compound 11, Compound 13, Compound 31, Compound 35, Compound 40, Compound 41, Compound 42, Compound 121, Compound 143, Compound 205, Compound 206, Compound 212, Compound 213, Compound 218, Compound 220, Compound 221, Compound 224, Compound 248, Compound 249, Compound 250, Compound 287, Compound 288, Compound 332 or Compound 350 with proquinazid, combinations of Compound 3, Compound 8, Compound 9, Compound 10, Compound 11, Compound 13, Compound 31, Compound 35, Compound 40, Compound 41, Compound 42, Compound 121, Compound 143, Compound 205, Compound 206, Compound 212, Compound 213, Compound 218, Compound 220, Compound 221, Compound 224, Compound 248, Compound 249, Compound 250, Compound 287, Compound 288, Compound 332 or Compound 350 with prothioconazole, combinations of Compound 3, Compound 8, Compound 9, Compound 10, Compound 11, Compound 13, Compound 31, Compound 35, Compound 40, Compound 41, Compound 42, Compound 121, Compound 143, Compound 205, Compound 206, Compound 212, Compound 213, Compound 218, Compound 220, Compound 221, Compound 224, Compound 248, Compound 249, Compound 250, Compound 287, Compound 288, Compound 332 or Compound 350 with tebuconazole, combinations of Compound 3, Compound 8, Compound 9, Compound 10, Compound 11, Compound 13, Compound 31, Compound 35, Compound 40, Compound 41, Compound 42, Compound 121, Compound 143, Compound 205, Compound 206, Compound 212, Compound 213, Compound 218, Compound 220, Compound 221, Compound 224, Compound 248, Compound 249, Compound 250, Compound 287, Compound 288, Compound 332 or Compound 350 with triticonazole, combinations of Compound 3, Compound 8, Compound 9, Compound 10, Compound 11, Compound 13, Compound 31, Compound 35, Compound 40, Compound 41, Compound 42, Compound 121, Compound 143, Compound 205, Compound 206, Compound 212, Compound 213, Compound 218, Compound 220, Compound 221, Compound 224, Compound 248, Compound 249, Compound 250, Compound 287, Compound 288, Compound 332 or Compound 350 with famoxadone, combinations of Compound 3, Compound 8, Compound 9, Compound 10, Compound 11, Compound 13, Compound 31, Compound 35, Compound 40, Compound 41, Compound 42, Compound 121, Compound 143, Compound 205, Compound 206, Compound 212, Compound 213, Compound 218, Compound 220, Compound 221, Compound 224, Compound 248, Compound 249, Compound 250, Compound 287, Compound 288, Compound 332 or Compound 350 with penthiopyrad, combinations of Compound 3, Compound 8, Compound 9, Compound 10, Compound 11, Compound 13, Compound 31, Compound 35, Compound 40, Compound 41, Compound 42, Compound 121, Compound 143, Compound 205, Compound 206, Compound 212, Compound 213, Compound 218, Compound 220, Compound 221, Compound 224, Compound 248, Compound 249, Compound 250, Compound 287, Compound 288, Compound 332 or Compound 350 with 3-(difluoromethyl)-1-methyl-N-(3′,4′,5′-trifluoro[1,1′-biphenyl]-2-yl)-1H-pyrazole-4-carboxamide, combinations of Compound 3, Compound 8, Compound 9, Compound 10, Compound 11, Compound 13, Compound 31, Compound 35, Compound 40, Compound 41, Compound 42, Compound 121, Compound 143, Compound 205, Compound 206, Compound 212, Compound 213, Compound 218, Compound 220, Compound 221, Compound 224, Compound 248, Compound 249, Compound 250, Compound 287, Compound 288, Compound 332 or Compound 350 with 5-ethyl-6-octyl-[1,2,4]triazole[1,5-a]pyrimidin-7-amine, and Compound 3, Compound 8, Compound 9, Compound 10, Compound 11, Compound 13, Compound 31, Compound 35, Compound 40, Compound 41, Compound 42, Compound 121, Compound 143, Compound 205, Compound 206, Compound 212, Compound 213, Compound 218, Compound 220, Compound 221, Compound 224, Compound 248, Compound 249, Compound 250, Compound 287, Compound 288, Compound 332 or Compound 350 with Initium®.

The control efficacy of compounds of this invention on specific pathogens is demonstrated in TABLE A below. The pathogen control protection afforded by the compounds is not limited, however, to the species described in Tests A-E below. Descriptions of the compounds are provided in Index Tables A-B below. The following abbreviations are used in the index table: Me is methyl, MeO is methoxy, CN is cyano, c-Pr is cyclopropyl and Ph is phenyl, “Cmpd. No.” means compound number, and “Ex.” stands for “Example” and is followed by a number indicating in which example the compound is prepared. In Index Tables A-B the numerical value reported in the column “AP⁺ (M+1)”, is the molecular weight of the observed molecular ion formed by addition of H⁺ (molecular weight of 1) to the molecule having the greatest isotopic abundance (i.e. M). The presence of molecular ions containing one or higher atomic weight isotopes of lower abundance (e.g., ³⁷Cl, ⁸¹Br) is not reported. The reported M+1 peaks were observed by mass spectrometry using atmospheric pressure chemical ionization (AP⁺).

INDEX TABLE A

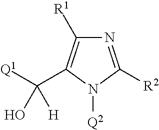

| | | | | | | AP⁺ |
|---|---|---|---|---|---|---|
| Cmpd. No. | R¹ | R² | Q¹ | Q² | m.p. (° C.) | (M + 1) |
| 1 (Ex. 1) | Cl | H | 2-Cl-4-F—Ph | 2,6-di-F—Ph | ** | ** |
| 2 (Ex. 2) | Cl | H | 2,4-di-F—Ph | 2-Cl-4-F—Ph | ** | ** |
| 3 | Me | H | 4-Cl—Ph | 2-Cl-4,6-di-F—Ph | — | 369 |
| 4 | Cl | Cl | 4-Cl—Ph | 2,6-di-F—Ph | — | 390 |
| 5 | Me | Cl | 2-Cl-4-F—Ph | 2-Br-4-F—Ph | — | 448 |
| 6 | Cl | Cl | 4-Cl—Ph | 2-Cl-6-F—Ph | — | 406 |
| 7 | Me | Cl | 4-Cl—Ph | 2-Cl-4,6-di-F—Ph | — | 402 |
| 8 | Me | Cl | 2,4-di-F—Ph | 2-Cl-4,6-di-F—Ph | — | 405 |
| 9 | Me | Cl | 2-Cl-4-F—Ph | 2-Cl-4,6-di-F—Ph | — | 423 |
| 11 | Me | Cl | 2,4-di-Cl—Ph | 2-Br-4-F—Ph | — | 465 |
| 12 | Me | H | 2,4-di-F—Ph | 2-Cl-4,6-di-F—Ph | — | 371 |
| 13 | Me | Cl | 2-Me-4-F—Ph | 2-Cl-4,6-di-F—Ph | — | 401 |
| 14 | Cl | Cl | 2-Me-4-F—Ph | 2-Cl-4,6-di-F—Ph | — | 421 |
| 15 | Me | H | 2-Me-4-F—Ph | 2-Cl-4,6-Ph | — | 367 |
| 16 | Me | Cl | 2,4-di-F—Ph | 2-Br-4,6-di-F—Ph | — | 450 |
| 17 | Me | Cl | 2,4-di-F—Ph | 2-Br-4,6-di-F—Ph | — | 450 |
| 18 | Cl | Cl | 2-Cl-3-Br-6-F—Ph | 2-Cl-4-F—Ph | — | 503 |
| 19 | Cl | Cl | 2,4-di-F—Ph | 2-Cl-4-F—Ph | — | 409 |
| 20 | Cl | Cl | 2,4-di-F—Ph | 2-Cl-4-F—Ph | — | 407 |
| 21 Note 1 | Cl | Cl | 2,4-di-F—Ph | 2,4-di-Cl—Ph | — | 424 |
| 22 Note 2 | Cl | Cl | 2,4-di-F—Ph | 2,4-di-Cl—Ph | — | 424 |
| 23 | Cl | Cl | 2-Me-4-F—Ph | 2-Cl-6-F—Ph | — | 404 |
| 24 | Me | Cl | 2,6-di-F—Ph | 2,6-di-F—Ph | * | * |
| 25 | Me | Cl | 2,6-di-F-3-Br—Ph | 2,6-di-F—Ph | * | * |
| 26 | Me | Cl | 2,4-di-F—Ph | 2,6-di-F—Ph | * | * |
| 27 | Me | Cl | 2,4-di-F—Ph | 2-Cl-4-F—Ph | — | 388 |
| 28 | Me | Cl | 2,4-di-F—Ph | 2-Cl-4-F—Ph | — | 388 |
| 30 | Me | Cl | 2,4,6-tri-F—Ph | 2-Cl-4-F—Ph | — | 407 |
| 31 | Cl | Cl | 2-Cl-4-F—Ph | 2,6-di-F—Ph | — | 406 |
| 32 | Cl | Cl | 2,4,6-tri-F—Ph | 2,6-di-F—Ph | — | 409 |
| 33 | Me | H | 2,4-di-Cl—Ph | 2-Cl-4-F—Ph | — | 387 |
| 34 | Me | Cl | 2-Cl-3-Br-6-F—Ph | 2-Cl-4-F—Ph | — | 483 |
| 35 | Me | Cl | 2-Cl-4-F—Ph | 2,4-di-F—Ph | — | 388 |
| 36 | Me | Cl | 2,6-di-F-4-Cl—Ph | 2,4-di-Cl—Ph | — | 439 |
| 37 | Me | Cl | 2-Cl-3-Br-6-F—Ph | 2,4-di-Cl—Ph | — | 500 |
| 38 | Me | Cl | 2,6-di-F-3-I—Ph | 2,4-di-F—Ph | — | 497 |
| 39 | Cl | Cl | 4-Cl—Ph | 2-Cl-4,6-di-F—Ph | — | 425 |
| 40 | Cl | Cl | 2,4-di-F—Ph | 2-Cl-4,6-di-F—Ph | — | 427 |
| 41 | Cl | Cl | 2-Cl-4-F—Ph | 2-Cl-6-F—Ph | — | 424 |
| 42 | Cl | Cl | 2-Cl-4-F—Ph | 2-Cl-4,6-di-F—Ph | — | 443 |
| 43 | Cl | Cl | 2-Me-4-F—Ph | 2,6-di-F—Ph | — | 387 |
| 44 | Cl | Cl | 4-Cl—Ph | 2-Br-4,6-di-F—Ph | — | 468 |
| 45 | Me | Cl | 2,4-di-F—Ph | 2-Br-4,6-di-F—Ph | — | 450 |
| 46 | Me | Cl | 2,6-di-F—Ph | 2-Cl-4-F—Ph | — | 388 |
| 47 | Cl | Cl | 4-Cl—Ph | 2-Cl-4-F—Ph | — | 406 |
| 48 | Cl | Cl | 4-Cl—Ph | 2-Cl-4-F—Ph | — | 406 |
| 49 | Cl | Cl | 2,4,6-tri-F—Ph | 2-Cl-4-F—Ph | — | 426 |
| 50 | Cl | Cl | 2-Me-4-F—Ph | 2-Cl-4-F—Ph | — | 404 |
| 51 | Cl | Cl | 2-Me-4-F—Ph | 2-Cl-4-F—Ph | — | 404 |
| 52 | Br | Me | 2-F-4-Me—Ph | 2,6-di-F—Ph | 199-200 | 413 |
| 53 | Br | Me | 2-F-4-MeO—Ph | 2,6-di-F—Ph | 191-192 | 429 |
| 54 | Cl | Me | 2-F-4-Cl—Ph | 2,6-di-F—Ph | 209-211 | 387 |
| 55 | Cl | Me | 2-F-4-Me—Ph | 2,6-di-F—Ph | 199-200 | |
| 56 | Cl | Me | 2-F-4-MeO—Ph | 2,6-di-F—Ph | 184-186 | |
| 57 | H | Me | 4-Cl—Ph | 2-Cl-4-F—Ph | — | 351 |
| 58 | Cl | Me | 2,4-F—Ph | 2-Cl-4-F—Ph | — | 386 |
| 59 | Cl | Me | 4-Cl—Ph | 2-Cl-4-F—Ph | — | 386 |
| 60 | Br | Me | 4-Cl—Ph | 2-Cl-4-F—Ph | — | 430 |
| 61 | H | Me | 2,4-di-Me—Ph | 2,6-di-F—Ph | 213-215 | — |
| 62 | H | Me | 2-Cl-4-MeO—Ph | 2,6-di-F—Ph | 202-204 | — |
| 63 | Br | H | 2,4-di-F—Ph | 2-Cl-4-F—Ph | * | * |
| 64 | Cl | Me | 2-Cl-4-F—Ph | 2-Cl-4,6-di-F—Ph | — | 423 |
| 65 | Br | Br | 2,4-di-F—Ph | 2-Cl-4-F—Ph | — | 497 |
| 66 | Cl | Me | 2,4-di-Cl—Ph | 2-Cl-4,6-di-F—Ph | — | 439 |
| 67 | Br | Me | 2-Br-4-F—Ph | 2-Br-6-F—Ph | — | 539 |
| 68 | Cl | Me | 2-Br-4-F—Ph | 2-Br-6-F—Ph | — | 493 |
| 69 | Br | Me | 2-Br-4-F—Ph | 2-Br-4,6-di-F—Ph | — | 557 |
| 70 | Cl | Me | 2-Br-4-F—Ph | 2-Br-4,6-di-F—Ph | — | 511 |
| 71 | Br | c-Pr | 2-Br-4-F—Ph | 2-Br-6-F—Ph | — | 565 |
| 72 | Cl | c-Pr | 2-Br-4-F—Ph | 2-Br-6-F—Ph | — | 519 |
| 73 | H | Me | 2-Cl-4-F—Ph | 2-Br-6-F—Ph | — | 415 |
| 74 | H | Me | 2-Cl-4-F—Ph | 2-Br-4,6-di-F—Ph | — | 433 |
| 75 | H | c-Pr | 2-Cl-4-F—Pr | 2-Br-6-F—Ph | — | 441 |
| 76 | Cl | Me | 2-Cl-4-F—Ph | 2,6-di-F-4-Br—Ph | — | 467 |
| 77 | Cl | Cl | 2-Me-4-F—Ph | 2,4-di-F—Ph | — | 389 |
| 78 Note 1 | Cl | Cl | 2,4-di-F—Ph | 2,4-di-F—Ph | — | 391 |
| 79 Note 2 | Cl | Cl | 2,4-di-F—Ph | 2,4-di-F—Ph | — | 391 |
| 80 | H | Me | 2-Me-4-F—Ph | 2,6-di-F—Ph | — | 333 |
| 83 | Me | Cl | 2,4-di-F—Ph | 2,4,6-tri-F—Ph | * | * |
| 84 | Cl | Cl | 2-Me-4-F—Ph | 2,4-di-Cl—Ph | — | 420 |
| 85 | Cl | Cl | 2-Me-4-F—Ph | 2,4-di-Cl—Ph | — | 420 |
| 86 | Cl | Cl | 4-Cl—Ph | 2,4-di-Cl—Ph | — | 422 |
| 87 | Cl | Cl | 4-Cl—Ph | 2,4-di-Cl—Ph | — | 422 |
| 88 | Br | Me | 2-Me-4-F—Ph | 2,6-di-F—Ph | 169-171 | 412 |
| 90 | Cl | Cl | 2,4,6-tri-F—Ph | 2,4-di-Cl—Ph | — | 442 |
| 91 | Cl | Me | 2-Me-4-F—Ph | 2,6-di-F—Ph | 151-153 | 367 |
| 92 | Cl | Me | 2,4-di-F—Ph | 2-Br-4,6-di-F—Ph | 169-171 | 451 |
| 93 | H | Me | 2-Br-4-F—Ph | 2-Br-6-F—Ph | — | 499 |
| 94 | H | Me | 2-Br-4-F—Ph | 2-Br-4,6-di-F—Ph | 151-153 | 477 |
| 95 | H | c-Pr | 2-Br-4-F—Ph | 2-Br-6-F—Ph | — | 485 |
| 96 | Br | H | 2,4-di-F—Ph | 2,6-di-F—Ph | 181-183 | 400 |
| 97 | H | Me | 2,4-di-F—Ph | 2,6-di-F—Ph | — | 337 |
| 98 | Cl | Me | 2-Cl-4-F—P | 2,6-di-F—Ph | 180-182 | 386 |
| 99 | Br | Me | 2-Cl-4-F—Ph | 2,6-di-F—Ph | 191-192.5 | 432 |
| 100 | Cl | Me | 2,4,6-tri-F—Ph | 2,6-di-F—Ph | 157-159 | 388 |
| 101 | Br | Me | 2,4,6-di-F—Ph | 2,6-di-F—Ph | 161.5-163 | — |
| 104 | Me | Cl | 2-Me-4-F—Ph | 2,4-di-F—Ph | — | 366 |
| 105 | Cl | Me | 2,6-di-F—Ph | 2,6-di-F—Ph | 184-186 | — |
| 106 | Br | Me | 2,6-di-F—Ph | 2,6-di-F—Ph | — | 416 |
| 107 | Br | Br | 2,4-di-F—Ph | 2-Cl-4,6-di-F—Ph | 106-108 | 515 |
| 108 | Br | Cl | 2,4-di-F—Ph | 2-Cl-4,6-di-F—Ph | 155-156 | — |
| 109 | H | Me | 2-Me-4-F—Ph | 2,6-di-F-4-Cl—Ph | 194-196 | 367 |
| 110 | Br | Me | 2-Me-4-F—Ph | 2,6-di-F-4-Cl—Ph | 197-198 | — |
| 111 | Cl | Me | 2-Me-4-F—Ph | 2,6-di-F-4-Cl—Ph | 166-167 | — |
| 112 | H | Me | 2,4-di-Cl—Ph | 2,6-di-F-4-Cl—Ph | 219-221 | — |
| 113 | Br | Me | 2,4-di-Cl—Ph | 2,6-di-F-4-Cl—Ph | 234-235 | — |
| 114 | Cl | Me | 2,4-di-Cl—Ph | 2,6-di-F-4-Cl—Ph | 207-208 | — |
| 115 | H | Me | 2-Cl-4-F—Ph | 2,6-di-F-4-Cl—Ph | 206-207 | — |
| 116 | Br | Me | 2-Cl-4-F—Ph | 2,6-di-F-4-Cl—Ph | 202-203 | — |
| 117 | Cl | Me | 2-Cl-4-F—Ph | 2,6-di-F-4-Cl—Ph | 194-195 | — |
| 118 | Cl | H | 2,4-di-F—Ph | 2-Cl-4,6-di-F—Ph | 161-163 | — |
| 119 | Br | H | 2,4-di-F—Ph | 2,6-di-F-4-Cl—Ph | 165-166 | — |
| 120 | Cl | H | 2,4-di-F—Ph | 2,6-di-F-4-Cl—Ph | 165-167 | — |
| 121 | Cl | Br | 2,4-di-F—Ph | 2-Cl-4,6-di-F—Ph | 142-144 | — |
| 122 | H | Me | 2,4-di-F—Ph | 2,6-di-F-4-Cl—Ph | 222-223 | — |
| 123 | Br | Br | 2,4-di-F—Ph | 2,6-di-F-4-Cl—Ph | 215-216 | — |
| 124 | Br | Cl | 2,4-di-F—Ph | 2,6-di-F-4-Cl—Ph | 201-202 | — |
| 125 | Cl | Br | 2,4-di-F—Ph | 2,6-di-F-4-Cl—Ph | 190-191 | — |
| 126 | Br | Me | 2,4-di-F—Ph | 2,6-di-F-4-Cl—Ph | 201-202 | — |
| 127 | Cl | Me | 2,4-di-F—Ph | 2,6-di-F-4-Cl—Ph | 204-205 | — |
| 128 | Cl | Me | 2,4,6-tri-F—Ph | 2-Cl-4-F—Ph | — | 406 |
| 129 | Cl | Me | 2-Cl-4-F—Ph | 2-Cl-4-F—Ph | — | 404 |
| 130 | Cl | Me | 2-Cl-4-F—Ph | 2-Br-4-F—Ph | — | 449 |
| 131 | Cl | Me | 2,4-di-F—Ph | 2,6-di-F—Ph | 184-186 | 371 |
| 132 | H | Me | 4-F—Ph | 2,6-di-F—Ph | — | 319 |
| 133 | Br | Me | 4-F—Ph | 2,6-F—Ph | — | 398 |
| 134 | Br | Me | 2-Cl-4-F—Ph | 2-Br-6-F—Ph | — | 493 |
| 135 | Cl | Me | 2-Cl-4-F—Ph | 2-Br-6-F—Ph | — | 440 |
| 136 | Br | Me | 2-Cl-4-F—Ph | 2-Br-4,6-di-F—Ph | — | 511 |
| 137 | Cl | Me | 2-Cl-4-F—Ph | 2-Br-4,6-F—Ph | — | 467 |
| 138 | H | Me | 2-Me-4-F—Ph | 2-Br-4-F—Ph | — | 394 |
| 139 | H | Me | 4-Cl—Ph | 2-Br-4-F—Ph | — | 396 |
| 140 | Br | Me | 2,6-di-F—Ph | 2-Cl-4-F—Ph | — | 432 |
| 141 | Br | Me | 2,4,6-tri-F—Ph | 2-Cl-4-F—Ph | — | 450 |
| 142 | Br | Me | 2-Cl-4-F—Ph | 2-Cl-4-F—Ph | — | 448 |
| 143 | Cl | Me | 2-Cl-4-MeO—Ph | 2,6-di-F—Ph | 181-183 | 399 |
| 144 | Br | Me | 2-Cl-4-MeO—Ph | 2,6-di-F—Ph | 201-203 | 444 |
| 145 | Br | Me | 2,4-di-Me—Ph | 2,6-di-F—Ph | 180-182 | 409 |
| 146 | H | Me | 2,6-di-F-4-MeO—Ph | 2,6-di-F—Ph | 142-144 | — |
| 147 | Br | Me | 2,6-di-F-4-MeO—Ph | 2,6-di-F—Ph | 208-210 | — |
| 148 | Br | Cl | 2,4-di-F—Ph | 2-Cl-4-F—Ph | — | 453 |
| 149 | Br | H | 2,4-di-F—Ph | 2-Cl-4,6-di-F—Ph | — | 437 |
| 150 | Br | c-Pr | 2-Cl-4-F—Ph | 2-Br-6-F—Ph | — | 519 |
| 151 | Cl | c-Pr | 2-Cl-4-F—Ph | 2-Br-6-F—Ph | — | 475 |
| 152 | H | c-Pr | 2,4-di-F—Ph | 2-Br-6-F—Ph | — | 425 |
| 153 | Cl | Me | 2,6-di-F-4-MeO—Ph | 2-Cl-4-F—Ph | — | 418 |
| 154 | Cl | Cl | 2,4-di-F—Ph | 2,6-di-F-4-Cl—Ph | 187-189 | 427 |
| 155 | Cl | Me | 2,4,6-tri-F—Ph | 2-Br-4-F—Ph | — | 450 |
| 156 | Cl | Me | 2,6-di-F-4-Cl—Ph | 2-Cl-4-F—Ph | — | 422 |
| 157 | H | Me | 2-Me-4-Cl—Ph | 2,6-di-F—Ph | — | 349 |
| 158 | H | Me | 2-F-4-Cl—Ph | 2,6-di-F—Ph | — | 353 |
| 159 | Cl | Me | 2-Me-4-Cl—Ph | 2,6-di-F—Ph | 162-164 | 383 |
| 160 | H | Me | 2-F-4-CN—Ph | 2,6-di-F—Ph | 211-212 | 344 |
| 161 | Br | Me | 2-Me-4-Cl—Ph | 2,6-di-F—Ph | 173-176 | — |
| 162 | Me | Cl | 2,4-di-F—Ph | 2,4-di-F—Ph | — | 370 |
| 163 | Me | Cl | 4-Cl—Ph | 2,4-di-F—Ph | — | 370 |
| 164 | Cl | Me | 2-Me-4-CN—Ph | 2,6-di-F—Ph | — | 373 |
| 165 | Br | Me | 2-Me-4-CN—Ph | 2,6-di-F—Ph | — | 419 |
| 166 | Cl | Me | 2-Cl-4-F—Ph | 2,4,6-tri-F—Ph | — | 404 |
| 167 (Ex. 4) | H | Me | 2-Cl-4-F—Ph | 2,4,6-tri-F—Ph | — | 371 |
| 168 (Ex. 5) | Br | Me | 2-Cl-4-F—Ph | 2,4,6-tri-F—Ph | — | 451 |
| 169 | Me | Me | 2-Cl-4-F—Ph | 2,4,6-tri-F—Ph | — | 384 |
| 170 | H | Me | 2,4,6-tri-F—Ph | 2-Cl-4-F—Ph | — | 370 |
| 171 | H | Me | 2-Cl-4-F—Ph | 2-Cl-4-F—Ph | — | 370 |
| 172 | Br | Me | 4-Cl—Ph | 2-Br-4-F—Ph | — | 474 |
| 173 | Br | Me | 2-Me-4-F—Ph | 2-Br-4-F—Ph | — | 472 |
| 174 | H | Me | 2,4,6-tri-F—Ph | 2-Br-4-F—Ph | — | 416 |
| 175 | H | Me | 2,4,6-tri-F—Ph | 2,4-di-F—Ph | — | 354 |
| 176 | Br | Me | 2,4,6-tri-F—Ph | 2-Br-4-F—Ph | — | 494 |
| 177 | Br | Me | 2,4,6-tri-F—Ph | 2,4-di-F—Ph | — | 433 |
| 178 | Br | Me | 2,4-di-F—Ph | 2,6-di-F—Ph | 169-171 | — |
| 179 | Br | Me | 2-F-4-Cl—Ph | 2,6-di-F—Ph | 206-208 | 433 |
| 180 | Cl | Me | 2-F-4-CN—Ph | 2,6-di-F—Ph | 252-254 | 378 |
| 181 | H | Me | 3-pyridinyl | 2,4,6-tri-F—Ph | — | 319 |
| 182 | Br | Me | 3-pyridinyl | 2,4,6-tri-F—Ph | — | 399 |
| 183 | Cl | Me | 2-Me-4-F—Ph | 2,6-di-F-4-MeO—Ph | — | 397 |
| 184 | H | Me | 2-Me-4-F—Ph | 2,6-di-F-4-MeO—Ph | * | * |
| 185 | H | Me | 2-Me-4-F—Ph | 2-Cl-6-F—Ph | 189-191 | — |
| 186 | Br | Me | 2-Me-4-F—Ph | 2-Cl-6-F—Ph | 179-181 | 429 |
| 187 | H | Me | 2-Cl-4-F—Ph | 2-Cl-6-F—Ph | — | 369 |
| 188 | Br | Me | 2-Cl-4-F—Ph | 2-Cl-6-F—Ph | 210-212 | — |
| 189 | H | Me | 2-Cl-4-F—Ph | 2-Br-4-F—Ph | — | 414 |
| 190 | Br | Me | 2-Cl-4-F—Ph | 2-Br-4-F—Ph | — | 492 |
| 191 | Cl | Me | 2,4-di-Me—Ph | 2,6-di-F—Ph | 169-171 | — |
| 192 | Cl | Me | 2,6-di-F-4-MeO—Ph | 2,6-di-F—Ph | 180-182 | 401 |
| 193 | Cl | Me | 2,6-di-Cl—Ph | 2,6-di-F—Ph | 210-212 | 405 |
| 194 | Br | Me | 2,6-di-Cl—Ph | 2,6-di-F—Ph | 217-219 | — |
| 195 | H | Me | 2,4-di-Cl—Ph | 2,6-di-F—Ph | 231-233 | — |
| 196 | H | Me | 2,4-di-Cl—Ph | 2-Cl-6-F—Ph | 203-205 | — |
| 197 | H | Me | 2-Cl-4-F—Ph | 2,6-di-F-4-CN—Ph | — | 378 |
| 198 | Cl | Me | 2-Cl-4-F—Ph | 2,6-di-F-4-CN—Ph | — | 412 |
| 199 | Br | Me | 2-F-4-CN—Ph | 2,6-di-F—Ph | 140-141 | — |
| 200 | Cl | Me | 2-Cl-4-F—Ph | 2-Cl-6-F—Ph | 209-210 | 405 |
| 201 | Br | Me | 2,4-di-Cl—Ph | 2-Cl-6-F—Ph | 133-135 | 465 |
| 202 | Br | Me | 2,4-di-Cl—Ph | 2,6-di-F—Ph | 211-212 | — |
| 203 | Br | Me | 2,4,6-tri-F—Ph | 2-Br-3,5-di-MeO—Ph | — | 536 |
| 204 | Cl | Me | 2,4,6-tri-F—Ph | 4-Cl—Ph | — | 386 |
| 205 | Me | Cl | 2,4-di-F—Ph | 2-Br-6-F—Ph | 189-190 | — |
| 206 | Me | Cl | 2-Cl-4-F—Ph | 2-Cl-6-F—Ph | — | 405 |
| 207 | Me | Cl | 2-Cl-4-F—Ph | 2-Br-6-F—Ph | 221-222 | — |
| 208 | Me | Cl | 2-Me-4-F—Ph | 2-Cl-6-F—Ph | 221-222 | — |
| 209 | Me | Cl | 2-Me-4-F—Ph | 2-Br-6-F—Ph | 214-215 | — |
| 210 | Me | Cl | 2-F-4-MeO—Ph | 2-Cl-6-F—Ph | — | 399 |
| 211 | Me | Cl | 2-F-4-MeO—Ph | 2-Br-6-F—Ph | 198-199 | — |
| 212 | Me | Cl | 2-Me-4-MeO—Ph | 2-Cl-6-F—Ph | 208-209 | — |
| 213 | Me | Cl | 2-Me-4-MeO—Ph | 2-Br-6-F—Ph | 208-209 | — |
| 214 | H | Me | 2-Cl-4-F—Ph | 2,4-di-MeO—Ph | — | 376 |
| 215 | Br | H | 2-Cl-4-F—Ph | 2-Cl-4-F—Ph | — | 434 |
| 216 | Cl | Br | 2-Cl-4-F—Ph | 2-Cl-4-F—Ph | — | 460 |
| 217 | Br | Me | 2-Cl-4-F—Ph | 2,4-di-MeO—Ph | — | 457 |
| 218 | Br | Cl | 2-Cl-4-F—Ph | 2-Cl-4-F—Ph | — | 468 |
| 219 | Me | Cl | 2-Cl-4-MeO—Ph | 2,4-di-Cl—Ph | 189-191 | — |
| 220 | Me | Cl | 2-Me-4-MeO—Ph | 2,6-di-F—Ph | 159-161 | — |
| 221 | Me | Cl | 2-Cl-4-MeO—Ph | 2,6-di-F—Ph | 170-172 | — |
| 222 | Br | H | 2-Me-4-MeO—Ph | 2-Cl-6-F—Ph | — | 427 |
| 223 | H | Me | 2-Cl-4-F—Ph | 2,6-di-Me—Ph | — | 345 |
| 224 | Br | Me | 2-Me-4-MeO—Ph | 2-Cl-6-F—Ph | 177-182 | 441 |
| 225 | Br | Me | 2-Cl-4-F—Ph | 2,6-di-Me—Ph | 226-227 | 425 |
| 226 | Cl | Me | 2-Cl-4-F—Ph | 2,6-di-Me—Ph | 203-204 | 379 |
| 227 | H | Me | 2,4,6-tri-F—Ph | 2,6-di-Cl—Ph | — | 387 |
| 228 | Cl | Me | 2,4,6-tri-F—Ph | 2,6-di-Cl—Ph | — | 422 |
| 229 | Br | H | 2-Me-4-MeO-5-Br—Ph | 2-Cl-4-F—Ph | — | 505 |
| 230 | Br | H | 2-F-4-MeO—Ph | 2-Cl-4-F—Ph | — | 430 |
| 231 | Cl | Me | 2-Cl-4-MeO—Ph | 2-Br-4,6-di-F—Ph | — | 478 |
| 232 | Br | H | 2,6-di-F-4-MeO—Ph | 2-Cl-4-F—Ph | — | 448 |
| 233 | Br | Br | 2-Cl-4-F—Ph | 2-Cl-4-F—Ph | — | 514 |
| 234 | Cl | H | 2,6-di-F-4-MeO—Ph | 2-Cl-4-F—Ph | — | 404 |
| 235 | Br | Br | 2,6-di-F-4-MeO—Ph | 2-Cl-4-F—Ph | — | 526 |
| 236 | Cl | Cl | 2-Cl-4-MeO—Ph | 4,6-di-F—Ph | — | 421 |
| 237 | Cl | Br | 2-Cl-4-MeO—Ph | 2,6-di-F—Ph | — | 465 |
| 238 | H | Me | 2-F-4-MeO—Ph | 2-Cl-6-F—Ph | — | 365 |
| 239 | H | Me | 2-Cl-4-MeO—Ph | 2-Cl-6-F—Ph | — | 381 |
| 240 | Br | Me | 2-Cl-4-F—Ph | 2,4-di-F—Ph | — | 432 |
| 241 | Cl | Me | 2-Cl-4-F—Ph | 2,4-di-F—Ph | — | 388 |
| 242 | H | Me | 4-Cl—Ph | 2,4-di-Cl—Ph | — | 368 |
| 243 | Br | Me | 4-Cl—Ph | 2,4-di-Cl—Ph | — | 446 |
| 244 | Cl | Me | 4-Cl—Ph | 2,4-di-Cl—Ph | — | 402 |
| 245 | Me | Me | 2,4-di-F—Ph | 2-Cl-4,6-di-F—Ph | 205-206 | 385 |
| 246 | Me | Me | 2,4-di-F—Ph | 2,6-di-F-4-Cl—Ph | — | 385 |
| 247 | Br | Br | 2-F-4-MeO—Ph | 2-Cl-4-F—Ph | — | 509 |
| 248 | Me | Cl | 2-Me-4-MeO—Ph | 2-Cl-4-F—Ph | — | 396 |
| 249 | Me | Cl | 2-Cl-4-MeO—Ph | 2-Cl-4-F—Ph | — | 415 |
| 250 | Me | Br | 2,4-di-F—Ph | 2-Cl-4,6-di-F—Ph | 186-188 | 451 |
| 251 | H | Me | 2-Br-4-F—Ph | 2-Cl-4-F—Ph | — | 414 |
| 252 | H | Me | 2-Cl-4-MeO—Ph | 2-Cl-F—Ph | — | 383 |
| 253 | Br | Me | 2-Br-4-F—Ph | 2-Cl-4-F—Ph | — | 492 |
| 254 | Br | Me | 2-Cl-4-MeO—Ph | 2-Cl-4-F—Ph | — | 460 |
| 255 | Cl | Me | 2-Cl-4-MeO—Ph | 2-Cl-4-F—Ph | — | 416 |
| 256 | H | Me | 2,4-di-Cl—Ph | 2,4-di-F—Ph | — | 370 |
| 257 | H | Me | 2,6-di-F-4-MeO—Ph | 2,4-di-F—Ph | — | 367 |
| 258 | Br | Me | 2,6-di-F-4-MeO—Ph | 2,4-di-F—Ph | — | 446 |
| 259 | Br | Me | 2,4-di-Cl—Ph | 2,4-di-F—Ph | — | 448 |
| 260 | Cl | H | 2-Cl-4-MeO—Ph | 2,6-di-F—Ph | — | 384 |
| 261 | Me | Cl | 2-Cl-4-MeO—Ph | 2-Cl-6-F—Ph | 198-200 | — |
| 262 | Me | Cl | 2-Me-4-Cl—Ph | 2-Cl-6-F—Ph | 211-212 | — |
| 263 | Me | Cl | 2-Me-4-Cl—Ph | 2-Br-6-F—Ph | 217-218 | — |
| 264 | Me | Cl | 2,4-di-F—Ph | 2-Cl—Ph | 208-210 | — |
| 265 | Me | Cl | 2,4-di-F—Ph | 2-Br—Ph | 195-197 | — |
| 266 | Me | Cl | 2-F-4-MeO—Ph | 2-Cl—Ph | 188-190 | — |
| 267 | Me | Cl | 2-F-4-MeO—Ph | 2-Br—Ph | — | 427 |
| 268 | Me | Cl | 2-Br-4-F—Ph | 2,6-di-F—Ph | 219-221 | — |
| 269 | Cl | Me | 2,6-di-F-4-MeO—Ph | 2,4-di-F—Ph | — | 401 |
| 270 | Cl | Me | 2,4-di-Cl—Ph | 2,4-di-F—Ph | — | 404 |
| 271 | H | Me | 2,6-di-F-4-Cl—Ph | 2,4-di-F—Ph | — | 370 |
| 272 | Cl | Me | 2-Br-4-F—Ph | 2-Cl-4-F—Ph | — | 448 |
| 273 | Me | Cl | 2,4-di-F—Ph | 2,6-di-F-4-Cl—Ph | 209-209 | 405 |
| 274 (Ex. 6) | Br | H | 2-Cl-4-MeO—Ph | 2,6-di-F—Ph | — | 431 |
| 275 | Br | Me | 2-F-4-MeO—Ph | 2-Cl-6-F—Ph | — | 445 |
| 276 | Br | Me | 2-Cl-4-MeO—Ph | 2-Cl-6-F—Ph | — | 461 |
| 277 | Cl | Me | 2-F-4-MeO—Ph | 2-Cl-6-F—Ph | — | 399 |
| 278 | Cl | Me | 2-Cl-4-MeO—Ph | 2-Cl-6-F—Ph | — | 417 |
| 279 | Br | Me | 2,4,6-tri-F—Ph | 3,5-di-MeO—Ph | 222-224 | 459 |
| 280 | H | Me | 2-MeO-4-F—Ph | 2,6-di-F—Ph | 205-207 | 349 |
| 281 | Br | Me | 2-MeO-4-F—Ph | 2,6-di-F—Ph | 143-145 | 426 |
| 282 | H | Me | 2-Cl-4-MeO—Ph | 2,4-di-F—Ph | — | 364 |
| 283 | H | Me | 2-F-4-MeO—Ph | 2,4-di-F—Ph | — | 348 |
| 284 | Br | Me | 2-F-4-MeO—Ph | 2,4-di-F—Ph | — | 428 |
| 285 | Br | Me | 2-Cl-4-MeO—Ph | 2,4-di-F—Ph | — | 444 |
| 286 | Br | Br | 2-Cl-4-MeO—Ph | 2,6-di-F—Ph | — | 509 |
| 287 (Ex. 7) | Br | Cl | 2-Cl-4-MeO—Ph | 2,6-di-F—Ph | — | 465 |
| 288 | Me | Cl | 2-Cl-4-F—Ph | 2,6-di-F—Ph | 208-209 | — |
| 289 | Me | Cl | 2-Cl-4-MeO—Ph | 2-Cl—Ph | 172-174 | — |
| 290 | Me | Cl | 2-Cl-4-MeO—Ph | 2-Br—Ph | 146-148 | — |
| 291 | Me | Cl | 2-Cl-4-F—Ph | 2-Cl—Ph | 207-208 | — |
| 292 | Me | Cl | 2-Cl-4-F—Ph | 2-Br—Ph | 155-157 | — |
| 293 | Me | Cl | 2-Me-4-MeO—Ph | 2-Cl—Ph | 181-183 | — |
| 294 | Me | Cl | 2-Me-4-MeO—Ph | 2-Br—Ph | 174-175 | — |
| 295 | Me | Cl | 2-Me-4-Cl—Ph | 2-Cl—Ph | 214-217 | — |
| 296 | Me | Cl | 2-Me-4-Cl—Ph | 2-Br—Ph | 207-208 | — |
| 297 | Me | Cl | 2-Me-4-F—Ph | 2-Cl—Ph | 215-217 | — |
| 298 | Me | Cl | 2-Me-4-F—Ph | 2-Br—Ph | 180-181 | — |
| 299 | Me | Br | 2,4-di-F—Ph | 2,6-di-F-4-Cl | 211-212 | 451 |
| 300 | H | Me | 2-F-4-MeO—Ph | 2-Cl-4-F—Ph | — | 367 |
| 301 | H | Me | 2-Br-4-F—Ph | 2,4-di-F—Ph | — | 398 |
| 302 | Br | Me | 2-F-4-MeO—Ph | 2-Cl-4-F—Ph | — | 444 |
| 303 | H | Me | 2-F-4-MeO—Ph | 2-Br-4-F—Ph | — | 410 |
| 304 | Cl | Me | 2-F-4-MeO—Ph | 2-Cl-4-F—Ph | — | 402 |
| 305 | H | Me | 2,6-di-F-4-Cl—Ph | 2-Br-4-F—Ph | — | 432 |
| 306 | Cl | Me | 2-F-4-MeO—Ph | 2,4-di-F—Ph | — | 382 |
| 307 | Cl | Me | 2-Cl-4-MeO—Ph | 2,4-di-F—Ph | — | 398 |
| 308 | Br | Me | 2,6-di-F-4-Cl—Ph | 2-Br-4-F—Ph | — | 510 |
| 309 | Br | Me | 2-Br-4-F—Ph | 2,4-di-F—Ph | — | 476 |
| 310 | Br | Me | 2-F-4-MeO—Ph | 2-Br-4-F—Ph | — | 488 |
| 311 | H | Me | 2-F-4-CN—Ph | 2-Br-4-F—Ph | — | 403 |
| 312 | H | Me | 2-Cl-4-MeO—Ph | 2-Br-4-F—Ph | — | 425 |
| 313 | Cl | Me | 2-Br-4-F—Ph | 2,4-di-F—Ph | — | 432 |
| 314 | Cl | Me | 2-F-4-MeO—Ph | 2-Br-4-F—Ph | — | 442 |
| 315 | Cl | Me | 2-Me-4-F—Ph | 2-Br-4,6-di-F—Ph | — | 447 |
| 316 | Cl | Me | 2,4-di-F—Ph | 2-Br-6-F—Ph | — | 433 |
| 317 | Br | Me | 2,4-di-F—Ph | 2-Cl-6-Me—Ph | — | 429 |
| 318 | H | Me | 2,4-di-F—Ph | 2,6-di-F-4-CN—Ph | * | * |
| 319 | Cl | Cl | 2-Cl-4-F—Ph | 2-F-4-MeO—Ph | — | 421 |
| 320 | Cl | Me | 2,4-di-F—Ph | 2-Cl-6-Me—Ph | — | 383 |
| 321 | H | Me | 2,4-di-F—Ph | 2-Br-4-F—Ph | — | 398 |
| 322 | H | Me | 2,6-di-F-4-MeO—Ph | 2-Br-4-F—Ph | — | 428 |
| 323 | Br | Me | 2,6-di-F-4-MeO—Ph | 2-Br-4-F—Ph | — | 506 |
| 324 | Br | Me | 2-Cl-4-MeO—Ph | 2-Br-4-F—Ph | — | 504 |
| 325 | Cl | Me | 2,6-di-F-4-MeO—Ph | 2-Br-4-F—Ph | — | 461 |
| 326 | Br | Me | 2-F-4-CN—Ph | 2-Br-4-F—Ph | — | 482 |
| 327 | Cl | Cl | 2,4,6-tri-F—Ph | 2-F-4-MeO—Ph | — | 422 |
| 328 | Cl | Cl | 2,4-di-F—Ph | 2-F-4-MeO—Ph | — | 403 |
| 329 | H | Me | 2-F-4-MeO—Ph | 2-Cl-4,6-di-F—Ph | — | 383 |
| 330 | H | Me | 2-F-4-MeO—Ph | 2-Br-4,6-di-F—Ph | — | 429 |
| 331 | H | Me | 2-Cl-4-MeO—Ph | 2-Cl-4,6-di-F—Ph | — | 399 |
| 332 | Me | Cl | 2,4-di-F—Ph | 2-Cl-6-F—Ph | — | 387 |
| 333 | Cl | Me | 2-Cl-4-MeO—Ph | 2-Br-4-F—Ph | — | 460 |
| 334 | Br | Me | 2,4-di-F—Ph | 2-Br-4-F—Ph | — | 476 |
| 335 | Br | Me | 2-Cl-4-F—Ph | 2-Br-6-Me—Ph | — | 489 |
| 336 | Cl | Me | 2-Cl-4-F—Ph | 2-Br-6-Me—Ph | — | 445 |
| 337 | Br | Me | 2,4-di-F—Ph | 2-Br-6-Me—Ph | — | 473 |
| 338 | Cl | Me | 2,4-di-F—Ph | 2-Br-6-Me—Ph | — | 429 |
| 339 | Cl | Me | 2,4-di-F—Ph | 2-F-6-Me—Ph | — | 367 |
| 340 | Cl | Me | 2-Cl-4-F—Ph | 2-F-6-Me—Ph | — | 383 |
| 341 | Br | Me | 2,4-di-F—Ph | 2-F-6-Me—Ph | — | 413 |
| 342 | Br | Me | 2-Cl-4-F—Ph | 2-F-6-Me—Ph | — | 429 |
| 343 | Br | Me | 2-Cl-4-F—Ph | 2-Cl-6-Me—Ph | — | 445 |
| 344 | Cl | Me | 2-Cl-4-F—Ph | 2-Cl-6-Me—Ph | — | 401 |
| 345 | Cl | Me | 2-F-4-MeO—Ph | 2-Cl-4,6-di-F—Ph | 178-180 | 417 |
| 346 | H | Me | 2-Cl-4-MeO—Ph | 2-Br-4,6-di-F—Ph | * | * |
| 347 | Br | Me | 2-F-4-MeO—Ph | 2-Cl-4,6-di-F—Ph | 192-194 | 463 |
| 348 | Br | Me | 2-F-4-MeO—Ph | 2-Br-4,6-di-F—Ph | 189-190 | |
| 349 | Br | Me | 2-Cl-4-MeO—Ph | 2-Cl-4,6-di-F—Ph | 198-200 | 479 |
| 350 | Cl | Me | 2-Cl-4-MeO—Ph | 2-Cl-4,6-di-F—Ph | 183-186 | — |
| 351 | Br | Me | 2-Cl-4-MeO—Ph | 2-Br-4,6-di-F—Ph | 182-185 | — |
| 352 | H | ClCH₂ | 2-Cl-4-MeO—Ph | 2-Br-4,6-di-F—Ph | — | 479 |
| 353 | Br | Me | 2,4,6-tri-F—Ph | 4-Cl—Ph | 432 | — |
| 354 | Br | H | 2-Cl-4-MeO—Ph | 2-Cl-4-F—Ph | — | 447 |
| 355 | Br | Br | 2-Cl-4-MeO—Ph | 2-Cl-4-F—Ph | — | 526 |
| 356 | Cl | H | 2-Cl-4-MeO—Ph | 2-Cl-4-F—Ph | — | 402 |
| 357 | Cl | Br | 2-Cl-4-MeO—Ph | 2-Cl-4-F—Ph | — | 481 |
| 358 | Br | Cl | 2-Cl-4-MeO—Ph | 2-Cl-4-F—Ph | — | 481 |
| 359 | H | Me | 2,6-di-F-4-Cl—Ph | 2-Cl-4-F—Ph | — | 389 |
| 360 | H | Me | 2-Cl-4-F—Ph | 2,4-di-F—Ph | — | 352 |
| 361 | H | Me | 2,4,6-tri-F—Ph | 2,4-di-Cl—Ph | — | 389 |
| 362 | Br | Me | 2,6-di-F-4-Cl—Ph | 2-Cl-4-F—Ph | — | 466 |
| 363 | H | Me | 2,6-di-F-4-MeO—Ph | 2-Cl-4-F—Ph | — | 382 |
| 364 | Br | Me | 2,6-di-F-4-MeO—Ph | 2-Cl-4-F—Ph | — | 462 |
| 365 | Br | Me | 2,4,6-tri-F—Ph | 2,4-di-Cl—Ph | — | 466 |
| 366 | Cl | Me | 2-Me-4-F—Ph | 2,6-di-Cl—Ph | — | 401 |
| 367 | Br | Me | 2-Me-4-F—Ph | 2,6-di-Cl—Ph | — | 445 |
| 368 | H | Me | 2-Me-4-F—Ph | 2-Cl-4,6-di-F—Ph | — | 367 |
| 369 | H | Me | 2,4-di-F—Ph | 2-Cl-4,6-di-F—Ph | — | 371 |
| 370 | H | Me | 2-Cl-4-F—Ph | 2-Cl-4,6-di-F—Ph | — | 387 |
| 371 | H | Me | 2,4-di-Cl—Ph | 2-Cl-4,6-di-F—Ph | — | 405 |
| 372 | Br | Me | 2-Me-4-F—Ph | 2-Cl-4,6-F—Ph | 181-183 | 447 |
| 373 | Cl | Me | 2-Me-4-F—Ph | 2-Cl-4,6-di-F—Ph | 189-191 | 401 |
| 374 | Br | Me | 2,4-di-F—Ph | 2-Cl-4,6-di-F—Ph | 165-167 | 451 |
| 375 | Br | Me | 2-Cl-4-F—Ph | 2-Cl-4,6-di-F—Ph | 162-164 | — |
| 376 | Br | Me | 2,4-di-Cl—Ph | 2-Cl-4,6-di-F—Ph | 202-204 | — |
| 377 | Cl | Me | 2,4-di-F—Ph | 2-Cl-4,6-di-F—Ph | 179-181 | — |
| 378 | H | Me | 4-Cl—Ph | 2,4-di-F—Ph | — | 334 |
| 379 | Br | Me | 2-Me-4-F—Ph | 2,4-di-F—Ph | — | 412 |
| 380 | Br | Me | 4-Cl—Ph | 2,4-di-F—Ph | — | 414 |
| 381 | H | Me | 2,4-di-F—Ph | 2,4-di-F—Ph | — | 336 |
| 382 | Br | Me | 2,4-di-F—Ph | 2,4-di-F—Ph | — | 417 |
| 383 | Cl | Me | 2-Br-4-F—Ph | 2,6-di-F—Ph | 174-176 | 432 |
| 384 | Br | Me | 2-Br-4-F—Ph | 2,6-di-F—Ph | 193-195 | 477 |
| 385 | H | Me | 2-Br-4-F—Ph | 2-Cl-6-F—Ph | 203-205 | — |
| 386 | Cl | Me | 2-Br-4-F—Ph | 2-Cl-6-F—Ph | 213-215 | — |
| 387 | Br | Me | 2-Br-4-F—Ph | 2-Cl-6-F—Ph | 219-221 | — |
| 388 | H | Me | 2,4-di-F—Ph | 2-Cl-6-F—Ph | 174-175 | — |
| 389 | Cl | Me | 2,4-di-F—Ph | 2-Cl-6-F—Ph | 174-177 | — |
| 390 | H | Me | 2-Cl-4-F—Ph | 2,6-di-F-4-MeO—Ph | — | 383 |
| 391 | B— | Me | 2-Me-4-F—Ph | 2,6-di-F-4-MeO—Ph | — | 441 |
| 392 | Br | Me | 2-Cl-4-F—Ph | 2,6-di-F-4-MeO—Ph | — | 463 |
| 393 | Br | Me | 2,4-di-F—Ph | 2-Br-6-F—Ph | — | 477 |
| 394 | Br | Me | 2,4-di-F—Ph | 2-Br-4,6-di-F—Ph | — | 495 |
| 395 | H | Me | 2-Me-4-F—Ph | 2-Cl-4-F—Ph | — | 348 |
| 396 | H | Me | 2,4-di-F—Ph | 2-Cl-4-F—Ph | — | 352 |
| 397 | Br | Me | 2-Me-4-F—Ph | 2-Cl-4-F—Ph | — | 428 |
| 398 | Cl | Me | 2-Me-4-F—Ph | 2-Cl-4-F—Ph | — | 382 |
| 399 | Br | Me | 2,4-di-F—Ph | 2-Cl-4-F—Ph | — | 432 |
| 400 | Br | Me | 2,4-di-F—Ph | 2-Cl-6-F—Ph | 184-186 | 432 |
| 401 | H | Me | 2-Me-4-CN—Ph | 2,6-di-F—Ph | — | 340 |
| 402 | H | Me | 2-F-4-Me—Ph | 2,6-di-F—Ph | — | 333 |
| 403 | Cl | Me | 2-Cl-4-F—Ph | 2,6-di-F-4-MeO—Ph | — | 417 |
| 404 | H | Me | 2,4-di-F—Ph | 2,6-di-F-4-MeO—Ph | * | * |
| 405 | H | Me | 2-Me-4-F—Ph | 2-Br-6-F—Ph | — | 395 |
| 406 | Br | Me | 2-Me-4-F—Ph | 2-Br-6-F—Ph | — | 473 |
| 407 | Cl | Me | 2-Me-4-F—Ph | 2-Br-6-F—Ph | — | 429 |
| 408 | Br | Me | 2-Me-4-F—Ph | 2-Br-4,6-di-F—Ph | — | 491 |
| 409 | H | Me | 2,4-di-F—Ph | 2-Br-6-F—Ph | — | 399 |
| 410 | H | Me | 2,4-di-F—Ph | 2-Br-4,6-di-F—Ph | — | 417 |
| 411 | Cl | Me | 2,4-di-F—Ph | 2,6-di-F-4-MeO—Ph | — | 401 |
| 412 | Br | Me | 2,4-di-F—Ph | 2,6-di-F-4-MeO—Ph | — | 446 |
| 413 | H | Me | 2-F-3-pyridinyl | 2,4,6-tri-F—Ph | — | 337 |
| 414 | Br | Me | 2-F-3-pyridinyl | 2,4,6-tri-F—Ph | | 418 |
| 415 | H | Me | 2-Me-4-F—Ph | 2,4-di-F—Ph | | 332 |

*See Index Table C for ¹H NMR data.
**See synthesis example for ¹H NMR.
Note 1:
enantiomer A.
Note 2:
enantiomer B.

INDEX TABLE B

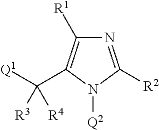

| | | | | | | | | AP⁺ |
|---|---|---|---|---|---|---|---|---|
| Cmpd. No. | R¹ | R² | R³ | R⁴ | Q¹ | Q² | m.p. (° C.) | (M + 1) |
| 10 | Cl | Cl | F | H | 2-Cl-4-F—Ph | 2-Cl-6-F—Ph | — | 426 |
| 29 | Me | Cl | OH | Me | 2,6-di-F—Ph | 2,6-di-F—Ph | * | * |
| 81 (Ex. 3) | Me | Cl | OH | Me | 2,4-di-F—Ph | 2-Cl-4,6-di-F—Ph | ** | ** |
| 82 | Me | Cl | OH | Me | 2,4-di-F—Ph | 2,4,6-tri-F—Ph | * | * |
| 89 | Cl | Cl | F | H | 2,4-di-F—Ph | 2-Cl-4-F—Ph | — | 409 |
| 102 | Me | Cl | MeC(═O)O | H | 2,4-di-F—Ph | 2,4,6-tri-F—Ph | 191-192.5 | — |
| 103 | Cl | Cl | F | H | 2,4,6-tri-F—Ph | 2,4-di-Cl—Ph | — | 444 |

*See Index Table C for ¹H NMR data.
**See synthesis example for ¹H NMR.

INDEX TABLE C

| Compd. | |
|---|---|
| No. | ¹H NMR Data (CDCl₃ solution unless indicated otherwise)^{a} |
| 24 | δ 7.46 (m, 1H), 7.23 (m, 1H), 7.01 (m, 2H), 6.79 (m, 2H), 5.95 |
| | (s, 1H), 3.08 (br s, 1H), 2.12 (s, 3H). |
| 25 | δ 7.44 (m, 2H), 7.03 (t, 1H), 6.95 (t, 1H), 6.70 (t, 1H), 6.01 |
| | (d, 1H), 3.94 (d, 1H), 2.22 (s, 3H). |
| 26 | δ 7.43 (m, 1H), 7.04 (m, 2H), 6.84 (t, 1H), 6.70 (t, 1H), 6.62 |
| | (t, 1H), 5.96 (s, 1H), 2.30 (br s, 1H), 2.24 (s, 3H). |
| 29 | δ 7.35 (m, 1H), 7.17 (m, 1H), 6.87 (t, 2H), 6.73 (m, 2H), 3.28 |
| | (t, 1H), 2.21 (s, 3H), 1.96 (t, 3H). |
| 63 | δ 7.55-7.45 (m, 1H), 7.33 (s, 1H), 7.20-6.58 (m, 5H), 6.18 |
| | and 6.08 (m, 1H, total), 2.85 (br s) and 2.62 (br s, 1H total). |
| 82 | δ 7.04 (m, 1H), 6.73 (m, 2H), 6.61 (m, 1H), 6.40 (tt, 1H), 2.48 |
| | (s, 1H), 2.40 (s, 3H), 1.95 (s, 3H). |
| 83 | δ 7.05 (q, 1H), 6.84 (m, 1H), 6.70 (m, 2H), 6.60 (m, 1H), 5.97 |
| | (s, 1H), 2.26 (s, 3H), 2.23 (br s, 1H). |
| 184 | (DMSO-d₆) δ 7.32 (m, 1H), 7.00-6.90 (m, 4H), 6.35 |
| | (s, 1H), 5.63 (m, 1H), 5.47 (m, 1H), 3.85 (s, 3H), 2.07 |
| | (s, 3H), 2.03 (s, 3H). |
| 318 | (DMSO-d₆) δ 8.10 (m, 2H), 7.31 (m, 1H), 7.15 (m, 1H), |
| | 7.02 (m, 1H), 6.58 (m, 1H), 5.99 (m, 1H), 5.66 (m, 1H), |
| | 2.06 (s, 3H). |
| 346 | δ 7.48-6.65 (m, 6H), 5.83 (m) and 5.78 (m, 1H total), 3.81 (s) |
| | and 3.79 (s, 3H total), 2.72 (br s) and 2.66 (br s, 1H total), 2.12 |
| | (s) and 2.10 (s, 3H total). |
| 404 | (DMSO-d₆) δ 7.35 (m, 1H), 7.11 (m, 1H), 7.02 (m, 1H), |
| | 6.95 (m, 2H), 6.55 (s, 1H), 5.88 (m, 1H), 5.60 (m, 1H), 3.85 |
| | (s, 3H), 2.02 (s, 3H). |

^{a1}H NMR data are in ppm downfield from tetramethylsilane. Couplings are designated by (s)—singlet, (d)—doublet, (t)—triplet, (q)—quartet, (m)—multiplet, (br s)—broad singlet and (tt)—triplet of triplets.

### Biological Examples of the Invention

General protocol for preparing test suspensions for Tests A-D: the test compounds were first dissolved in acetone in an amount equal to 3% of the final volume and then suspended at the desired concentration (in ppm) in acetone and purified water (50/50 mix by volume) containing 250 ppm of the surfactant Trem® 014 (polyhydric alcohol esters). The resulting test suspensions were then used in Tests A-D. Spraying a 200 ppm test suspension to the point of run-off on the test plants was the equivalent of a rate of 800 g/ha.

### Test A

The test suspension was sprayed to the point of run-off on wheat seedlings. The following day the seedlings were inoculated with a spore suspension of Puccinia recondite f. sp. tritici (the causal agent of wheat leaf rust) and incubated in a saturated atmosphere at 20° C. for 24 h, and then moved to a growth chamber at 20° C. for 7 days, after which time visual disease ratings were made.

### Test B

The test suspension was sprayed to the point of run-off on wheat seedlings. The following day the seedlings were inoculated with a spore suspension of Septoria tritici (the causal agent of wheat leaf blotch) and incubated in a saturated atmosphere at 24° C. for 48 h, and then moved to a growth chamber at 20° C. for 19 days, after which time visual disease ratings were made.

### Test C

The test suspension was sprayed to the point of run-off on tomato seedlings. The following day the seedlings were inoculated with a spore suspension of Botrytis cinerea (the causal agent of tomato Botrytis) and incubated in a saturated atmosphere at 20° C. for 48 h, and then moved to a growth chamber at 24° C. for 3 days, after which time visual disease ratings were made.

### Test D

The test suspension was sprayed to the point of run-off on wheat seedlings. The following day the seedlings were inoculated with a spore dust of Blumeria graminis f. sp. tritici, (also known as Erysiphe graminis f. sp. tritici, the causal agent of wheat powdery mildew) and incubated in a growth chamber at 20° C. for 8 days, after which time visual disease ratings were made.

### Test E

The test suspension was sprayed to the point of run-off on wheat seedlings. The following day the seedlings were inoculated with a spore suspension of Septoria nodorum (the causal agent of Septoria glume blotch) and incubated in a saturated atmosphere at 24° C. for 48 h, and then moved to a growth chamber at 20° C. for 9 days, after which time visual disease ratings were made.

Results for Tests A-E are given in Table A. In the Table, a rating of 100 indicates 100% disease control and a rating of 0 indicates no disease control (relative to the controls). A dash (—) indicates no test results. An asterisk “*” next to the rating value indicates a 40 ppm test suspension was used.

TABLE A

| Cmpd. No | Test A | Test B | Test C | Test D |
|---|---|---|---|---|
| 1 | 100 | 100 | 99 | 99 |
| 2 | 100 | 100 | 100 | 81 |
| 3 | 89 | 96 | 99 | 0 |
| 4 | 99* | 100* | 99* | 99* |
| 5 | 97 | 93 | 37 | — |
| 6 | 99 | 100 | 100 | 99 |
| 7 | 93 | 100 | 100 | 85 |
| 8 | 100 | 100 | 100 | 100 |
| 9 | 100 | 100 | 100 | 100 |
| 10 | 100 | 100 | 100 | 96 |
| 11 | 99 | 68 | 31 | 0 |
| 12 | 99 | 100 | 95 | 21 |
| 13 | 100 | 100* | 99 | 100 |
| 14 | 100 | 100* | 99 | 100 |
| 15 | 0 | 0* | 15 | 0 |
| 16 | 100 | 100 | 100 | 100 |
| 17 | 100 | 100 | 100 | 100 |
| 18 | 100 | 100 | 100 | 91 |
| 19 | 100 | 100 | 100 | 98 |
| 20 | 100 | 100 | 100 | 100 |
| 21 | 100 | 100 | 100 | 69 |
| 22 | 100 | 100 | 98 | 0 |
| 23 | 100 | 100 | 100 | 100 |
| 24 | 100* | 100* | 100* | 91* |
| 25 | 99* | 100* | 87* | 0* |
| 26 | 100* | 100* | 100* | 99* |
| 27 | 100 | 100 | 100 | 99 |
| 28 | 100 | 100 | 100 | 71 |
| 29 | 98* | 91* | 99* | 0* |
| 30 | 100 | 100 | 100 | 100 |
| 31 | — | — | — | — |
| 32 | — | — | — | — |
| 33 | 100 | 100 | 100 | 0* |
| 34 | 99 | 98 | 99 | — |
| 35 | 100 | 100 | 100 | 68 |
| 36 | 99 | 100 | 98 | 0 |
| 37 | 98 | 100 | 91 | 21 |
| 38 | 99 | 95 | 7 | 0 |
| 39 | 100 | 100 | 99 | 99 |
| 40 | 100 | 100 | 100 | 100 |
| 41 | 100 | 100 | 100 | 100 |
| 42 | 100 | 100 | 100 | 100 |
| 43 | 100 | 100 | 100 | 100 |
| 44 | 99 | 100 | 99 | 88 |
| 45 | — | — | — | — |
| 46 | 100 | 100 | 100 | 100 |
| 47 | 100 | 100 | 99 | 0 |
| 48 | 100 | 100 | 100 | 85 |
| 49 | 100 | 100 | 100 | 100 |
| 50 | 100 | 100 | 100 | 90 |
| 51 | 100 | 100 | 99 | 47 |
| 52 | 99* | 100* | 37* | 35-* |
| 53 | 100* | 100* | 99* | 43* |
| 54 | 98* | 100* | 80* | 69* |
| 55 | 100* | 100* | 98* | 98* |
| 56 | 100* | 100* | 100* | 13* |
| 57 | 96 | 100 | 34 | 0 |
| 58 | 100 | 100 | 100 | 72 |
| 59 | 100 | 100 | 99 | 73 |
| 60 | 100 | 100 | 99 | 73 |
| 61 | — | — | — | — |
| 62 | — | — | — | — |
| 63 | 96 | 59 | 98 | 0 |
| 64 | 100* | 100* | 99* | 93* |
| 65 | 100* | 100* | 100* | 64* |
| 66 | 100* | 100* | 99* | 69* |
| 67 | 100 | 100 | 99 | 90 |
| 68 | 100 | 100 | 100 | 96 |
| 69 | 100 | 100 | 99 | 90 |
| 70 | 100* | 100* | 99* | 43* |
| 71 | 100 | 100 | 0 | 13 |
| 72 | 79 | 100 | 0 | 43 |
| 73 | 100 | 100 | 99 | 0 |
| 74 | 100 | 100 | 100 | 0 |
| 75 | 94 | 1 | 0 | 0 |
| 76 | 99* | 100* | 0* | 0* |
| 77 | 100 | 100 | 94 | 95 |
| 78 | 100 | 100 | 99 | 82 |
| 79 | 100 | 100 | 98 | 0 |
| 80 | 99 | 100 | 0 | 0 |
| 81 | — | — | — | — |
| 82 | 89* | 100* | 50* | 0* |
| 83 | 100* | 100* | 99* | 100* |
| 84 | 96 | 100 | 0 | 0 |
| 85 | 99 | 100 | 88 | 87 |
| 86 | 96 | 100 | 73 | 56 |
| 87 | 91 | 100 | 60 | 48 |
| 88 | 99 | 100 | 96 | 90 |
| 89 | 100 | 100 | 98 | 96 |
| 90 | 100 | 100 | 98 | 99 |
| 91 | 100 | 100 | 98 | 94 |
| 92 | 100 | 100 | 99 | 95 |
| 93 | 100 | 94 | 70 | 0 |
| 94 | 100 | 100 | 99 | 56 |
| 95 | 0 | 0 | 0 | 0 |
| 96 | 68* | 100* | 40* | 0* |
| 97 | 95* | 100* | 0* | 0* |
| 98 | 100* | 100* | 100* | 99* |
| 99 | 100* | 100* | 99* | 98* |
| 100 | 100* | 100* | 97* | 82* |
| 101 | 99* | 100* | 81* | 87* |
| 102 | 100* | 100* | 97* | 69* |
| 103 | 100 | 100 | 100 | 92 |
| 104 | 100 | 100 | 99 | 97 |
| 105 | 99* | 100* | 85* | 64* |
| 106 | 99* | 100* | 91* | 43* |
| 107 | 100 | 100 | 100 | 94 |
| 108 | 100* | 100* | 67* | 99* |
| 109 | 99* | 94* | 0* | 0* |
| 110 | 96* | 100* | 0* | 0* |
| 111 | 98* | 100* | 0* | 0* |
| 112 | 74* | 100* | 0* | 0* |
| 113 | 95* | 100* | 0* | 0* |
| 114 | 99* | 100* | 0* | 0* |
| 115 | 100* | 96* | 97* | 0* |
| 116 | 99* | 100* | 0* | 0* |
| 117 | 99* | 100* | 0* | 0* |
| 118 | 100* | 100* | 91* | 48 |
| 119 | 98* | 100* | 97* | 0* |
| 120 | 98* | 100* | 99* | 0* |
| 121 | 100* | 100* | 100* | 98* |
| 122 | 98* | 100* | 0* | 0* |
| 123 | 98* | 100* | 95* | 0* |
| 124 | 100* | 100* | 100* | 0* |
| 125 | 100* | 100* | 100* | 79* |
| 126 | 96* | 100* | 0* | 0* |
| 127 | 100* | 100* | 73* | 0* |
| 128 | 100 | 100 | 99 | 0 |
| 129 | 100 | 100 | 98 | 0 |
| 130 | 99 | 100 | 87* | 0 |
| 131 | 100* | 100* | 99* | 99* |
| 132 | 99 | 100 | 41 | 0 |
| 133 | 100* | 100* | 100* | 64* |
| 134 | 100 | 100 | 100 | 98 |
| 135 | 100 | 100 | 100 | 98 |
| 136 | 100 | 100 | 100 | 79 |
| 137 | 100* | 100* | 100* | 87* |
| 138 | 89 | 28 | 0 | 0 |
| 139 | 86 | 49 | 0 | 0 |
| 140 | 100 | 100 | 100 | 50 |
| 141 | 100 | 100 | 99 | 96 |
| 142 | 100 | 100 | 99 | 71 |
| 143 | 100* | 100* | 99* | 99* |
| 144 | 100* | 100* | 100* | 99* |
| 145 | 100* | 100* | 51* | 98* |
| 146 | — | — | — | |
| 147 | 100* | 100* | 99* | 0* |
| 148 | — | — | — | — |
| 149 | — | — | — | — |
| 150 | 85 | 100 | 0 | 27 |
| 151 | 74 | 100 | 0 | 64 |
| 152 | 74 | 0 | 0 | 0 |
| 153 | 100 | 100 | 99* | 94 |
| 154 | 100* | 100* | 100* | 89* |
| 155 | 100 | 100 | 99 | 0 |
| 156 | 98 | 92 | 16 | 0 |
| 157 | — | — | — | — |
| 158 | — | — | — | — |
| 159 | 99* | 100* | 99* | 97* |
| 160 | — | — | — | — |
| 161 | 92* | 100* | 94* | 84* |
| 162 | 100 | 100 | 100 | 98 |
| 163 | 98 | 100 | 100 | 13 |
| 164 | 93* | 100* | 0* | 0* |
| 165 | 80* | 100* | 8* | 13* |
| 166 | 100* | 100* | 100* | 43* |
| 167 | 100 | 100 | 33 | 0 |
| 168 | 100* | 100* | 99* | 56* |
| 169 | 100* | 100* | 40* | 0* |
| 170 | 99 | 73 | 63 | 0 |
| 171 | 100 | 100 | 95 | 0 |
| 172 | 100 | 100 | 100 | 90 |
| 173 | 99 | 100 | 16 | 48 |
| 174 | 88 | 35 | 0 | 0 |
| 175 | 97 | 3 | 9 | 0 |
| 176 | 100 | 100 | 100 | 96 |
| 177 | 100 | 100 | 99 | 0 |
| 178 | 100* | 100* | 96* | 64* |
| 179 | 68* | 100* | 78* | 43* |
| 180 | 0* | 48* | 0* | 0* |
| 181 | 0 | 1 | 0 | 0 |
| 182 | 68 | 100 | 0 | 21 |
| 183 | 86* | 100* | 8* | 0* |
| 184 | — | — | — | — |
| 185 | — | — | — | — |
| 186 | 99* | 100* | 99* | 97* |
| 187 | — | — | — | — |
| 188 | 100* | 100* | 99* | 99* |
| 189 | 100 | 100 | 99 | 27 |
| 190 | 100 | 100 | 99 | 0 |
| 191 | 100* | 100* | 80* | 99* |
| 192 | 100* | 100* | 100* | 53* |
| 193 | 99* | 100* | 59* | 0* |
| 194 | 99* | 100* | 77* | 0* |
| 195 | — | — | — | — |
| 196 | — | — | — | — |
| 197 | — | — | — | — |
| 198 | 97* | 100* | 0* | 0* |
| 199 | 0* | 62* | 0* | 0* |
| 200 | 100* | 100* | 99* | 98* |
| 201 | 100* | 100* | 99* | 89* |
| 202 | 98* | 100* | 100* | 82* |
| 203 | 41 | 30 | 66 | 0 |
| 204 | 19 | 75 | 45 | 0 |
| 205 | 99* | 100* | 100* | 93* |
| 206 | 100* | 100* | 100* | 93* |
| 207 | 100* | 100* | 100* | 81* |
| 208 | 100* | 100* | 99* | 81* |
| 209 | 99* | 100* | 100* | 42* |
| 210 | 100 | 100* | 100* | 99* |
| 211 | 100* | 100* | 100* | 98* |
| 212 | 100* | 100* | 100* | 97* |
| 213 | 100* | 100* | 100* | 91* |
| 214 | 0* | 0* | 0* | 0* |
| 215 | 92* | 85* | 55* | 0* |
| 216 | 99* | 100* | 100* | 91* |
| 217 | 0* | 0* | 42* | 0* |
| 218 | 100* | 100* | 100* | 89* |
| 219 | 99* | 100* | 98* | 21* |
| 220 | 100* | 100* | 100* | 97* |
| 221 | 100* | 100* | 99* | 97* |
| 222 | 91* | 57* | 30* | 0* |
| 223 | — | — | — | — |
| 224 | 100* | 100* | 100* | 100* |
| 225 | 89 | 83 | 37 | 0 |
| 226 | 94 | 98 | 6 | 0 |
| 227 | 84 | 83 | 40 | 0 |
| 228 | — | — | — | — |
| 229 | 68* | 36* | 0* | 0* |
| 230 | 97* | 50* | 99* | 0* |
| 231 | 100* | 100* | 99* | 98* |
| 232 | 100* | 59* | — | 0* |
| 233 | 100* | 100* | 95* | 72* |
| 234 | 99* | 79* | 26* | 43* |
| 235 | 100* | 100* | 100* | 99* |
| 236 | 100* | 100* | 100* | 99* |
| 237 | 100* | 100* | 100* | 98* |
| 238 | — | — | — | — |
| 239 | — | — | — | — |
| 240 | 99* | 100* | 99* | 89* |
| 241 | 100* | 100* | 99* | 21* |
| 242 | 94* | 50* | 29* | 0* |
| 243 | 98* | 95* | 91* | 79* |
| 244 | 98* | 100* | 78* | 21* |
| 245 | 100* | 100* | 91* | 84* |
| 246 | 100* | 100* | 37* | 0* |
| 247 | — | — | — | — |
| 248 | — | — | — | — |
| 249 | — | — | — | — |
| 250 | 100* | 100* | 100* | 100* |
| 251 | 100 | 93 | 98 | 0 |
| 252 | 100 | 96 | 74 | 48 |
| 253 | 100 | 100 | 100 | 27 |
| 254 | 100 | 100 | 100 | 83 |
| 255 | 100 | 96 | 8 | 0 |
| 256 | 55 | 6 | 0 | 0 |
| 257 | 92 | 24 | 0 | 0 |
| 258 | 100 | 95 | 79 | 0 |
| 259 | 99 | 100 | 0 | 0 |
| 260 | 100 | 100 | 100 | 96 |
| 261 | — | — | — | — |
| 262 | — | — | — | — |
| 263 | — | — | — | — |
| 264 | — | — | — | — |
| 265 | — | — | — | — |
| 266 | — | — | — | — |
| 267 | — | — | — | — |
| 268 | — | — | — | — |
| 269 | 100* | 96* | 66* | 0* |
| 270 | 95* | 69* | 0* | 0* |
| 271 | 0* | 17* | 0* | 0* |
| 272 | 100* | 100* | 93* | 21* |
| 273 | 100* | 100* | 99* | 0* |
| 274 | 100 | 97 | 0 | 64 |
| 275 | 100* | 100* | 100* | 99* |
| 276 | 100* | 100* | 100* | 100* |
| 277 | 100* | 100* | 100* | 97* |
| 278 | 100* | 100* | 100* | 97* |
| 279 | 0* | 0* | 0* | 0* |
| 280 | — | — | — | — |
| 281 | 92* | 0* | 0* | 0* |
| 282 | 92* | 63* | 0* | 0* |
| 283 | 41* | 0* | 0* | 0* |
| 284 | 100* | 100* | 41* | 0* |
| 285 | 99* | 88* | 0* | 0* |
| 286 | 100* | 100* | 100* | 97* |
| 287 | 100* | 100* | 100* | 97* |
| 288 | — | — | — | — |
| 289 | — | — | — | — |
| 290 | — | — | — | — |
| 291 | — | — | — | — |
| 292 | — | — | — | — |
| 293 | — | — | — | — |
| 294 | — | — | — | — |
| 295 | — | — | — | — |
| 296 | — | — | — | — |
| 297 | — | — | — | — |
| 298 | — | — | — | — |
| 299 | 100* | 100* | 100* | 87* |
| 300 | 95* | 95* | 0* | 0* |
| 301 | 97* | 19* | 24* | 0* |
| 302 | 100* | 100* | 100* | 64* |
| 303 | 74* | 43* | 8* | 0* |
| 304 | 100* | 100* | 99* | 0* |
| 305 | 9* | 17* | 21* | 0* |
| 306 | 100* | 100* | 97* | 0* |
| 307 | 99* | 100* | 0* | 0* |
| 308 | 100* | 100* | 100* | 93* |
| 309 | 100* | 100* | 65* | 64* |
| 310 | 100* | 100* | 100* | 56* |
| 311 | 9* | 5* | 0* | 0* |
| 312 | 28* | 12* | 0* | 0* |
| 313 | 100* | 100* | 17* | 0* |
| 314 | 100* | 100* | 99* | 47* |
| 315 | 99* | 100* | 96* | 96* |
| 316 | 100* | 100* | 99* | 95* |
| 317 | 100* | 89* | 96* | 56* |
| 318 | — | — | — | — |
| 319 | 100* | 100* | 99* | 0* |
| 320 | 99* | 100* | 96* | 79* |
| 321 | 0* | 0* | 0* | 0* |
| 322 | 28* | 2* | 0* | 0* |
| 323 | 100* | 100* | 100* | 99* |
| 324 | 100* | 94* | 80* | 69* |
| 325 | 100* | 98* | 100* | 92* |
| 326 | 99* | 90* | 30* | 0* |
| 327 | 100* | 100* | 100* | 29* |
| 328 | 100* | 100* | 100* | 64* |
| 329 | — | — | — | — |
| 330 | — | — | — | — |
| 331 | — | — | — | — |
| 332 | 100* | 100* | 99* | 92* |
| 333 | 95* | 72* | 99* | 86* |
| 334 | 99* | 100* | 99* | 64* |
| 335 | 98* | 86* | 35* | 64* |
| 336 | 97* | 89* | 72* | 90* |
| 337 | 86* | 86* | 71* | 0* |
| 338 | 94* | 93* | 70* | 56* |
| 339 | 98* | 98* | 86* | 76* |
| 340 | 99* | 100* | 96* | 89* |
| 341 | 94* | 100* | 86* | 43* |
| 342 | 94* | 100* | 98* | 64* |
| 343 | 96* | 100* | 84* | 69* |
| 344 | 99* | 95* | 54* | 72* |
| 345 | 100* | 100* | 99* | 35* |
| 346 | — | — | — | — |
| 347 | 100* | 100* | 82* | 69* |
| 348 | 100* | 100* | 98* | 90* |
| 349 | 100* | 100* | 98* | 90* |
| 350 | 100* | 100* | 98* | 98* |
| 351 | 100* | 100* | 90* | 95* |
| 352 | 100 | 100 | 99 | 95 |
| 353 | 9 | 44 | 28 | 0 |
| 354 | 94* | 18* | 0* | 0* |
| 355 | 100* | 100* | 100* | 97* |
| 356 | 95* | 19* | 0* | 0* |
| 357 | 100* | 100* | 99* | 98* |
| 358 | 100* | 100* | 100* | 99* |
| 359 | 68 | 0 | 0 | 0 |
| 360 | 99 | 93 | 67 | 0 |
| 361 | 95 | 15 | 9 | 0 |
| 362 | 99 | 85 | 0 | 0 |
| 363 | 98 | 74 | 9 | 0 |
| 364 | 100 | 100 | 100 | 95 |
| 365 | 100 | 100 | 100 | 64 |
| 366 | 99 | 100 | 100 | 97 |
| 367 | 98 | 100 | 96 | 96 |
| 368 | 100 | 100 | 99 | 0 |
| 369 | — | — | — | — |
| 370 | 100 | 100 | 100 | 0 |
| 371 | 95 | 100 | 58 | 0 |
| 372 | — | — | — | — |
| 373 | — | — | — | — |
| 374 | — | — | — | — |
| 375 | — | — | — | — |
| 376 | — | — | — | — |
| 377 | — | — | — | — |
| 378 | 37 | 74 | 0 | 0 |
| 379 | 100 | 100 | 99 | 79 |
| 380 | 98 | 100 | 100 | 0 |
| 381 | 88 | 97 | 0 | 0 |
| 382 | 100 | 100 | 99 | 0 |
| 383 | 100* | 100* | 99* | 92* |
| 384 | 100* | 100* | 65* | 72* |
| 385 | — | — | — | — |
| 386 | 100* | 100* | 100* | 73* |
| 387 | 100* | 100* | 99* | 90 |
| 388 | — | — | — | — |
| 389 | 100* | 100* | 100* | 98 |
| 390 | — | — | — | — |
| 391 | 99* | 100* | 0* | 0* |
| 392 | 99* | 100* | 0* | 0* |
| 393 | 100 | 100 | 100 | 97 |
| 394 | 100 | 100 | 100 | 72 |
| 395 | 99 | 100 | 21 | 35 |
| 396 | 100 | 100 | 87 | 0 |
| 397 | 100 | 100 | 100 | 73 |
| 398 | 100 | 100 | 46 | 0 |
| 399 | 100 | 100 | 99 | 90 |
| 400 | 100* | 100* | 100* | 100* |
| 401 | — | — | — | — |
| 402 | — | — | — | — |
| 403 | 99* | 100* | 8* | 0* |
| 404 | 98 | | 0 | 0 |
| 405 | 99 | 94 | 9 | 0 |
| 406 | 100 | 100 | 43 | 56 |
| 407 | 99 | 100 | 52 | 0 |
| 408 | 100 | 100 | 99 | 56 |
| 409 | 99 | 100 | 57 | 0 |
| 410 | 100 | 100 | 97 | 13 |
| 411 | 100* | 100* | 85* | 50* |
| 412 | 100* | 100* | 0* | 0* |
| 413 | 88 | 98 | 0 | 0 |
| 414 | 100 | 100 | 67 | 13 |
| 415 | 74 | 18 | 0 | 0 |

What is claimed is:

1. A compound selected from Formula 1, N-oxides and salts thereof, 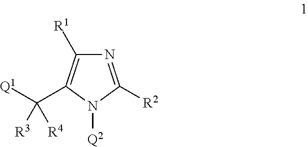
wherein
Q¹ is a phenyl ring substituted with 1 to 4 substituents independently selected from R^{5a}; or a thienyl, pyrazolyl, imidazolyl, thiazolyl, pyridinyl, pyridazinyl or pyrimidinyl ring or a quinazolinyl ring system, each ring or ring system optionally substituted with up to 4 substituents independently selected from R^{5a} on carbon atom ring members and R^{5b} on nitrogen atom ring members;
Q² is a phenyl ring substituted with 1 to 4 substituents independently selected from R^{5a}; or a thienyl, pyrazolyl, imidazolyl, thiazolyl, pyridinyl, pyridazinyl or pyrimidinyl ring or a quinazolinyl ring system, each ring or ring system optionally substituted with up to 4 substituents independently selected from R^{5a} on carbon atom ring members and R^{5b} on nitrogen atom ring members;
R¹ and R² are each independently H, halogen, cyano, nitro, C₁-C₃ alkyl, C₂-C₃ alkenyl, C₂-C₃ alkynyl, C₁-C₃ haloalkyl, C₂-C₃ haloalkenyl, cyclopropyl, halocyclopropyl, C₁-C₃ hydroxyalkyl, C₂-C₃ cyanoalkyl, C₂-C₃ alkoxyalkyl, C₁-C₃ alkoxy, C₁-C₃ haloalkoxy, C₁-C₃ alkylthio or C₁-C₃ haloalkylthio;
R³ is halogen, —OR⁶ or —SC≡N;
R⁴ is H or C₁-C₆ alkyl;
each R^{5a} is independently halogen, cyano, hydroxy, nitro, C₁-C₃ alkyl, C₂-C₃ alkenyl, C₂-C₃ alkynyl, C₁-C₃ haloalkyl, C₂-C₃ haloalkenyl, cyclopropyl, halocyclopropyl, C₂-C₃ cyanoalkyl, C₁-C₃ alkylthio, C₁-C₃ haloalkylthio, C₁-C₃ alkylsulfinyl, C₁-C₃ haloalkylsulfinyl, C₁-C₃ alkylsulfonyl, C₁-C₃ haloalkylsulfonyl, C₁-C₃ alkoxy, C₁-C₃ haloalkoxy, C₂-C₄ alkylcarbonyloxy, C₂-C₃ alkylcarbonyl, C₁-C₃ alkylamino, C₂-C₄ dialkylamino, C₂-C₃ alkylcarbonylamino, C₃-C₆ trialkylsilyl, —CH(═O), —NHCH(═O), —C(═S)NH₂, —SC≡N or -T-U—V;
each R^{5b} is independently cyano, C₁-C₃ alkyl, C₂-C₃ alkenyl, C₂-C₃ alkynyl, C₁-C₃ haloalkyl, cyclopropyl, C₂-C₃ alkoxyalkyl, C₂-C₃ alkylaminoalkyl, C₃-C₄ dialkylaminoalkyl, C₁-C₃ alkoxy, C₂-C₃ alkylcarbonyl or C₂-C₃ alkoxycarbonyl;
R⁶ is H, —CH(═O), C₁-C₆ alkyl, C₂-C₆ alkenyl, C₃-C₆ alkynyl, C₁-C₆ haloalkyl, C₃-C₆ cycloalkyl, C₃-C₆ halocycloalkyl, C₂-C₆ alkoxyalkyl, C₂-C₆ cyanoalkyl, C₂-C₆ alkylcarbonyl, C₂-C₆ alkoxycarbonyl, C₂-C₆ (alkylthio)carbonyl, C₄-C₈ cycloalkylcarbonyl, C₄-C₈ cycloalkoxycarbonyl, C₄-C₈ (cycloalkylthio)carbonyl, C₂-C₆ alkoxy(thiocarbonyl) or C₄-C₈ cycloalkoxy(thiocarbonyl);
each T is independently O, S(═O)ₙ, N(R⁷) or a direct bond;
each U is independently C₁-C₆ alkylene, C₂-C₆ alkenylene, C₃-C₆ alkynylene, C₃-C₆ cycloalkylene or C₃-C₆ cycloalkenylene, wherein up to 3 carbon atoms are independently selected from C(═O), each optionally substituted with up to 5 substituents independently selected from halogen, cyano, nitro, hydroxy, C₁-C₆ alkyl, C₁-C₆ haloalkyl, C₁-C₆ alkoxy and C₁-C₆ haloalkoxy;
each V is independently cyano, N(R^{8a})(R^{8b}), OR⁹ or S(═O)ₙR⁹;
each R⁷ is independently H, C₁-C₆ alkyl, C₁-C₆ haloalkyl, C₂-C₆ alkylcarbonyl, C₂-C₆ alkoxycarbonyl, C₂-C₆ (alkylthio)carbonyl, C₂-C₆ alkoxy(thiocarbonyl), C₄-C₈ cycloalkylcarbonyl, C₄-C₈ cycloalkoxycarbonyl, C₄-C₈ (cycloalkylthio)carbonyl or C₄-C₈ cycloalkoxy(thiocarbonyl);
each R^{8a} and R^{8b} is independently H, C₁-C₆ alkyl, C₁-C₆ haloalkyl, C₂-C₆ alkenyl, C₃-C₆ alkynyl, C₃-C₆ cycloalkyl, C₃-C₆ halocycloalkyl, C₂-C₆ alkylcarbonyl, C₂-C₆ alkoxycarbonyl, C₂-C₆ (alkylthio)carbonyl, C₂-C₆ alkoxy(thiocarbonyl), C₄-C₈ cycloalkylcarbonyl, C₄-C₈ cycloalkoxycarbonyl, C₄-C₈ (cycloalkylthio)carbonyl or C₄-C₈ cycloalkoxy(thiocarbonyl); or
a pair of R^{8a} and R^{8b} are taken together with the nitrogen atom to which they are attached to form a 4- to 7-membered heterocyclic ring, the ring optionally substituted with up to 5 substituents independently selected from R¹⁰;
each R⁹ is independently H, C₁-C₆ alkyl, C₁-C₆ haloalkyl, C₂-C₆ alkenyl, C₃-C₆ alkynyl, C₃-C₆ cycloalkyl, C₃-C₆ halocycloalkyl, C₂-C₆ alkylcarbonyl, C₂-C₆ alkoxycarbonyl, C₂-C₆ (alkylthio)carbonyl, C₂-C₆ alkoxy(thiocarbonyl), C₄-C₈ cycloalkylcarbonyl, C₄-C₈ cycloalkoxycarbonyl, C₄-C₈ (cycloalkylthio)carbonyl or C₄-C₈ cycloalkoxy(thiocarbonyl);
each R¹⁰ is independently halogen, C₁-C₆ alkyl, C₁-C₆ haloalkyl or C₁-C₆ alkoxy;
each n is independently 0, 1 or 2;
provided that:
(a) when Q¹ and Q² are both phenyl substituted with 1 to 4 substituents independently selected from R^{5a}, then at least one R^{5a} substituent is attached at an ortho position; and
(b) when R¹ is H, then R² is other than H.

2. A compound of claim 1 wherein:
Q¹ is a phenyl ring substituted with 1 to 3 substituents independently selected from R^{5a}; or a pyridinyl or pyrimidinyl ring optionally substituted with up to 3 substituents independently selected from R^{5a};
Q² is a phenyl ring substituted with 1 to 3 substituents independently selected from R^{5a}; or a pyrazolyl, pyridinyl or pyrimidinyl ring optionally substituted with up to 3 substituents independently selected from R^{5a} on carbon atom ring members and methyl on the nitrogen atom ring member;
R¹ and R² are each independently H, halogen, cyano, C₁-C₃ alkyl or cyclopropyl;
R³ is Br, Cl, F, —OR⁶ or —SC≡N;
R⁴ is H or methyl;
each R^{5a} is independently halogen, cyano, C₁-C₂ alkyl, C₁-C₂ haloalkyl, cyclopropyl, C₁-C₂ alkoxy, C₁-C₂ alkylthio or -T-U—V;
R⁶ is H, —CH(═O), C₁-C₃ alkyl, C₁-C₂ haloalkyl, C₂-C₃ alkoxyalkyl, C₂-C₄ cyanoalkyl, C₂-C₄ alkylcarbonyl, C₂-C₄ alkoxycarbonyl, C₂-C₄ (alkylthio)carbonyl or C₂-C₄ alkoxy(thiocarbonyl);
each T is independently O, NH or a direct bond;
each U is independently C₁-C₃ alkylene, wherein up to 1 carbon atom is selected from C(═O);
each V is independently N(R^{8a})(R^{8b}) or OR⁹;
each R^{8a} and R^{8b} is independently H or methyl; and
each R⁹ is independently H, methyl or halomethyl.

3. A compound of claim 2 wherein
Q¹ is a phenyl ring substituted with 1 to 3 substituents independently selected from R^{5a};
Q² is a phenyl ring substituted with 1 to 3 substituents independently selected from R^{5a};
R¹ and R² are each independently H, Cl, Br, I or C₁-C₂ alkyl; and
each R^{5a} is independently halogen, cyano, methyl, halomethyl, cyclopropyl, methoxy, methylthio or -T-U—V.

4. A compound of claim 3 wherein
R¹ and R² are each independently Cl, Br or methyl;
R³ is —OR⁶;
R⁴ is H; and
R⁶ is H, —CH(═O)₅ C₁-C₃ alkyl, C₁-C₂ haloalkyl, C₂-C₃ alkoxyalkyl, C₂-C₄ cyanoalkyl, C₂-C₄ alkylcarbonyl or C₂-C₄ alkoxycarbonyl.

5. A compound of claim 4 wherein
each R^{5a} is independently Br, Cl, F, cyano or methoxy;
R⁶ is H; and
one of the Q¹ and Q² rings is substituted with 2 or 3 substituents and the other of the Q¹ and Q² rings is substituted with 1 or 2 substituents.

6. A compound of claim 1 which is selected from the group consisting of:
2,4-dichloro-α-(2-chloro-4-fluorophenyl)-1-(2,6-difluorophenyl)-1H-imidazole-5-methanol;
2-chloro-α-(2-chloro-4-fluorophenyl)-1-(2,6-difluorophenyl)-4-methyl-1H-imidazole-5-methanol;
2-bromo-α-(2-chloro-4-fluorophenyl)-1-(2,6-difluorophenyl)-4-methyl-1H-imidazole-5-methanol;
4-bromo-2-chloro-α-(2-chloro-4-fluorophenyl)-1-(2,6-difluorophenyl)-1H-imidazole-5-methanol;
2,4-dichloro-α-(2-chloro-4-fluorophenyl)-1-(2-chloro-6-fluorophenyl)-1H-imidazole-5-methanol;
2-chloro-α-(2-chloro-4-fluorophenyl)-1-(2-chloro-6-fluorophenyl)-4-methyl-1H-imidazole-5-methanol;
2-bromo-α-(2-chloro-4-fluorophenyl)-1-(2-chloro-6-fluorophenyl)-4-methyl-1H-imidazole-5-methanol;
4-bromo-2-chloro-α-(2-chloro-4-fluorophenyl)-1-(2-chloro-6-fluorophenyl)-1H-imidazole-5-methanol;
2,4-dichloro-1-(2-chloro-4,6-difluorophenyl)-α-(2-chloro-4-fluorophenyl)-1H-imidazole-5-methanol;
2-chloro-1-(2-chloro-4,6-difluorophenyl)-α-(2-chloro-4-fluorophenyl)-4-methyl-1H-imidazole-5-methanol;
2-bromo-1-(2-chloro-4,6-difluorophenyl)-α-(2-chloro-4-fluorophenyl)-4-methyl-1H-imidazole-5-methanol;
4-bromo-2-chloro-1-(2-chloro-4,6-difluorophenyl)-α-(2-chloro-4-fluorophenyl)-1H-imidazole-5-methanol;
1-(2-bromo-4,6-difluorophenyl)-2,4-dichloro-α-(2-chloro-4-fluorophenyl)-1H-imidazole-5-methanol;
1-(2-bromo-4,6-difluorophenyl)-2-chloro-α-(2-chloro-4-fluorophenyl)-4-methyl-1H-imidazole-5-methanol;
2-bromo-1-(2-bromo-4,6-difluorophenyl)-α-(2-chloro-4-fluorophenyl)-4-methyl-1H-imidazole-5-methanol;
4-bromo-1-(2-bromo-4,6-difluorophenyl)-2-chloro-α-(2-chloro-4-fluorophenyl)-1H-imidazole-5-methanol;
2,4-dichloro-α-(2-chloro-4-fluorophenyl)-1-(2,4-difluorophenyl)-1H-imidazole-5-methanol;
2-chloro-α-(2-chloro-4-fluorophenyl)-1-(2,4-difluorophenyl)-4-methyl-1H-imidazole-5-methanol;
2-bromo-α-(2-chloro-4-fluorophenyl)-1-(2,4-difluorophenyl)-4-methyl-1H-imidazole-5-methanol;
4-bromo-2-chloro-α-(2-chloro-4-fluorophenyl)-1-(2,4-difluorophenyl)-1H-imidazole-5-methanol;
2,4-dichloro-α,1-bis(2-chloro-4-fluorophenyl)-1H-imidazole-5-methanol;
2-chloro-α,1-bis(2-chloro-4-fluorophenyl)-4-methyl-1H-imidazole-5-methanol;
2-bromo-α,1-bis(2-chloro-4-fluorophenyl)-4-methyl-1H-imidazole-5-methanol;
4-bromo-2-chloro-α,1-bis(2-chloro-4-fluorophenyl)-1H-imidazole-5-methanol;
1-(2-bromo-4-fluorophenyl)-2,4-dichloro-α-(2-chloro-4-fluorophenyl)-1H-imidazole-5-methanol;
1-(2-bromo-4-fluorophenyl)-2-chloro-α-(2-chloro-4-fluorophenyl)-4-methyl-1H-imidazole-5-methanol;
2-bromo-1-(2-bromo-4-fluorophenyl)-α-(2-chloro-4-fluorophenyl)-4-methyl-1H-imidazole-5-methanol;
4-bromo-1-(2-bromo-4-fluorophenyl)-2-chloro-α-(2-chloro-4-fluorophenyl)-1H-imidazole-5-methanol;
2-bromo-4-chloro-1-(2-chloro-4,6-difluorophenyl)-α-(2,4-difluorophenyl)-1H-imidazole-5-methanol;
2-bromo-1-(2-chloro-4,6-difluorophenyl)-α-(2,4-difluorophenyl)-4-methyl-1H-imidazole-5-methanol;
2-chloro-α-(2-chloro-4-methoxyphenyl)-1-(2,6-difluorophenyl)-4-methyl-1H-imidazole-5-methanol;
2-chloro-1-(2,6-difluorophenyl)-α-(4-methoxy-2-methylphenyl)-4-methyl-1H-imidazole-5-methanol;
2-chloro-1-(2-chloro-6-fluorophenyl)-α-(4-methoxy-2-methylphenyl)-4-methyl-1H-imidazole-5-methanol;
4-bromo-2-chloro-α-(2-chloro-4-methoxyphenyl)-1-(2,6-difluorophenyl)-1H-imidazole-5-methanol;
4-bromo-1-(2-chloro-6-fluorophenyl)-α-(4-methoxy-2-methylphenyl)-2-methyl-1H-imidazole-5-methanol;
4-chloro-1-(2-chloro-4,6-difluorophenyl)-α-(2-chloro-4-methoxyphenyl)-2-methyl-1H-imidazole-5-methanol;
4-chloro-α-(2-chloro-4-methoxyphenyl)-1-(2,6-difluorophenyl)-2-methyl-1H-imidazole-5-methanol;
2-chloro-1-(2-chloro-4,6-difluorophenyl)-α-(2,4-difluorophenyl)-4-methyl-1H-imidazole-5-methanol;
2-chloro-1-(2-chloro-4,6-diflorophenyl)-α-(2-chloro-4-fluorophenyl)-4-methyl-1H-imidazole-5-methanol;
2-chloro-1-(2-chloro-4,6-difluorophenyl)-α-(4-fluoro-2-methylphenyl)-4-methyl-1H-imidazole-5-methanol;
2-chloro-1-(2-chloro-4-fluorophenyl)-α-(2-chloro-4-methoxyphenyl)-4-methyl-1H-imidazole-5-methanol;
2-chloro-1-(2-chloro-4-fluorophenyl)-α-(4-methoxy-2-methylphenyl)-4-methyl-1H-imidazole-5-methanol;
4-bromo-2-chloro-α,1-bis(2-chloro-4-fluorophenyl)-1H-imidazole-5-methanol;
2,4-dichloro-1-(2-chloro-4,6-difluorophenyl)-α-(2,4-difluorophenyl)-1H-imidazole-5-methanol;
2-chloro-1-(2-chloro-6-fluorophenyl)-α-(2,4,-difluorophenyl)-4-methyl-1H-imidazole-5-methanol;
1-(2-bromo-6-fluorophenyl)-2-chloro-α-(2,4-difluorophenyl)-4-methyl-1H-imidazole-5-methanol and
1-(2-bromo-6-fluorophenyl)-2-chloro-α-(4-methoxy-2-methylphenyl)-4-methyl-1H-imidazole-5-methanol.

7. A fungicidal composition comprising (a) a compound of claim 1; and (b) at least one other fungicide.

8. A fungicidal composition comprising (a) a compound of claim 1; and (b) at least one additional component selected from the group consisting of surfactants, solid diluents and liquid diluents.

9. A method for controlling plant diseases caused by fungal plant pathogens comprising applying to the plant or portion thereof, or to the plant seed, a fungicidally effective amount of a compound of claim 1.

10. A method for controlling plant diseases caused by fungal plant pathogens comprising applying to the plant or portion thereof, or to the plant seed, a fungicidally effective amount of a compound of claim 1.
